(19) United States
(12) Patent Application Publication
(10) Pub. No.: US 2016/0270822 A1
(43) Pub. Date: Sep. 22, 2016

(54) EXTERNAL FIXATION SYSTEM

(71) Applicant: Biomet Manufacturing, LLC, Warsaw, IN (US)

(72) Inventors: Jeffery Cresina, Warsaw, IN (US); Paul Thomas Slagle, Warsaw, IN (US); Kevin T. Stone, Winona Lake, IN (US); Jacy C. Hoeppner, Warsaw, IN (US)

(21) Appl. No.: 15/072,790

(22) Filed: Mar. 17, 2016

Related U.S. Application Data
(60) Provisional application No. 62/134,333, filed on Mar. 17, 2015.

(51) Int. Cl. A61B 17/62; A61B 17/64; A61B 17/66

(52) U.S. Cl. CPC A61B 17/62; A61B 17/66; A61B 17/6425; A61B 17/645

(57) ABSTRACT

Disclosed is an external fixation system. The external fixation system can comprise a ring frame, a strut, and a pivot housing connecting the strut to the ring frame. The pivot housing can comprise a first compression member, a second compression member, and a post assembly. The first compression member can be attached to the ring frame. The second compression member can be spaced from the first compression member and attached to the first compression member. The post assembly can be connected to the strut via an attachment post protruding from a spherical component and passing through the first compression member. The spherical component can be compressed between the first compression member and the second compression member to retain the post assembly in a fixed position.

### PRIORITY APPLICATIONS

This application claims the benefit of priority to U.S. Patent Application Ser. No. 62/134,333, filed Mar. 17, 2015, the content of which is hereby incorporated by reference in its entirety.

### TECHNICAL FIELD

The present teachings are related to an orthopedic external fixation system, and more particularly an external fixation system having universal articulation units for positioning fixation components, including rings, pins, rods, bars and posts relative to a patient's anatomy.

### BACKGROUND OF THE DISCLOSURE

The statements in this section merely provide background information related to the present disclosure and should not be construed as constituting prior art.

In various orthopedic surgical procedures, it is often necessary to secure or stabilize two or more portions of bone or soft tissue relative to one another. This need is often the result of a bone or soft tissue injury, such as an acute fracture of the bone. To ensure that the damaged bone fragments are capable of properly regenerating, it is important that the bone fragments be adequately stabilized during the regeneration process. To adequately stabilize the injured bone fragments and/or soft tissue, a bone distraction frame is typically installed onto the patient.

Once a distraction frame has been installed onto a patient, it is sometimes necessary to further adjust the frame to fine tune the alignment of the damaged bone fragments or soft tissue. This process, which is referred to as “fracture reduction,” is typically performed under the guidance of a C-arm (X-ray) and involves the surgeon manually pulling on the transfixation pin until the bones are aligned in a desired orientation. Once the surgeon is satisfied with this alignment, the clamps of the distraction frame can then be tightened.

While many external fixation devices have proven generally effective for stabilizing bones, these conventional systems are often difficult and time consuming to adjust once assembled, particularly as the surgeon may need to manually loosen and retighten the clamps several times during the fracture reduction process. Not only is the adjustment process time consuming, but the health and safety of the surgeon is also potentially compromised, particularly as the surgeon must expose his hands to the X-ray field during the reduction process. Thus, it would be useful to have an external fixation framing system that is not only easy to assemble, but also provides the surgeon with a greater degree of flexibility in terms of safely adjusting the frame once it has been assembled.

The present application is intended to improve upon and resolve some of these known deficiencies of the art.

### SUMMARY

To better illustrate the reaming apparatus disclosed herein, a non-limiting list of examples is provided here:

Example 1 can include an external fixation system. The external fixation system can comprise a ring frame, a strut, and a pivot housing connecting the strut to the ring frame. The pivot housing can comprise a first compression member, a second compression member, and a post assembly. The first compression member can be attached to the ring frame. The second compression member can be spaced from the first compression member and attached to the first compression member. The post assembly can be connected to the strut via an attachment post protruding from a spherical component and passing through the first compression member. The spherical component can be compressed between the first compression member and the second compression member to retain the post assembly in a fixed position.

In Example 2, the external fixation system of Example 1 can optionally include a socket configured to rotate the second compression member. The socket can include an internal structure that can mate with external features of the second compression member to enable rotation of the second compression member when the socket rotates.

In Example 3, the external fixation system of any one of or any combination of Examples 1 and 2 can optionally include the first compression member including a platform extending from a first surface, the platform sized to receive a clamping assembly.

In Example 4, the external fixation system of any one of or any combination of Examples 1-3 can optionally include a locking member movable from a first position to a second position. When the locking member is in the first position the post assembly can be pivotable within a space defined by the first compression member and the second compression member. When the locking member is in the second position the compression force can be applied to the spherical component.

In Example 5, the external fixation system of any one of or any combination of Examples 1-4 can optionally include the first compression member being connected to the second compression member via a plurality of threaded members.

In Example 6, the external fixation system of any one of or any combination of Examples 1-5 can optionally include a crossbar and a first distraction handle and the second distraction handle. The crossbar can be connected to the ring frame via a pair of distraction extension arms sized to space the crossbar a fixed distance from the ring frame. The first distraction handle and the second distraction handle can be connected to the crossbar and configured to allow a user to reposition the external fixation system once attached to a patient.

In Example 7, the external fixation system of claim 6 can optionally include the first distraction handle and the second distraction handle each being pivotably connected to the crossbar.

Example 8 can include an external fixation system. The external fixation system can comprise a proximal ring, a distal ring frame, a first frame strut, a first pivot housing, and a second pivot housing. The proximal ring frame can receive a first set of one or more fixation elements. The distal ring frame can receive a second set of one or more fixation elements. The first frame strut can fix a first relative position between the proximal ring frame and the distal ring frame. The first pivot housing can pivotably couple the proximal ring frame and the first frame strut. The second pivot housing can pivotably couple the distal ring frame and the first frame strut.

In Example 9, the external fixation system of Example 8 can optionally include a second frame strut, a third pivot housing, and a fourth pivot housing. The second frame strut can fix a second relative position between the proximal ring frame and the distal ring frame. The third pivot housing can pivotably couple the proximal ring frame and the second frame strut. The fourth pivot housing can pivotably couple the distal ring frame and the second frame strut.

In Example 10, the external fixation system of any one of or any combination of Examples 8 and 9 can optionally include the first pivot housing and the second pivot housing each comprise a post assembly. The post assembly can be connected to the first frame strut and a locking arm located between a first ring and a second ring. The locking arm can be configured to lock the post assembly in a fixed position.

In Example 11, the external fixation system of any one of or any combination of Examples 8-10 can optionally include the proximal ring frame or the distal ring frame including a platform extending from a first surface. The platform can be sized to receive a clamping assembly.

In Example 12, the external fixation system of any one of or any combination of Examples 8-11 can optionally include the first frame strut including a ratcheting member configured to fix a length of the first frame strut.

In Example 13, the external fixation system of any one of or any combination of Examples 8-12 can optionally include a cross bar, a first distraction handle, and a second distraction handle. The crossbar can be connected to the proximal ring frame or the distal ring frame via a pair of distraction extension arms. The first distraction handle and the second distraction handle can be connected to the crossbar.

In Example 14, the external fixation system of Example 13 can optionally include the first distraction handle and the second distraction handle each being pivotably connected to the crossbar.

Example 15 can include an external fixation system. The external fixation system can comprise a proximal ring frame, a distal ring frame, a first pivot housing, and a second pivot housing. The first pivot housing can pivotably connect the proximal ring frame to a first frame strut. The second pivot housing can pivotably connect the distal ring frame to the first frame strut. Each of the first pivot housing and the second pivot housing can include a clamp body and a locking arm. The clamp body can be sized to fit onto the proximal ring frame or the distal ring frame. The locking arm can be connected to the clamp body and pivotable about a point on the clamp body. When the locking arm is in a first position the clamp body can be slideable along a portion of the proximal ring frame or the distal ring frame. When the locking arm is in a second position a portion of the clamp body can contact the proximal ring frame or the distal ring frame to secure the clamp body in a fixed location.

In Example 16, the external fixation system of Example 15 can optionally include the frame strut being selected from a plurality of frame struts, each of the plurality of frame struts having a different size.

In Example 17, the external fixation system of any one of or any combination of Examples 15 and 16 can optionally include the frame strut being polyaxially connected to the proximal ring frame and the distal ring frame via the first pivot housing and the second pivot housing.

In Example 18, the external fixation system of any one of or any combination of Examples 15-17 can optionally include a crossbar, a first distraction handle, and a second distraction handle. The crossbar can be connected to the proximal ring frame or the distal ring frame via a pair of distraction extension arms that space the cross bar from the proximal ring frame or the distal ring frame a fixed distance. The first distraction handle and the second distraction handle can be connected to the crossbar. The first distraction handle and the second distraction handle can be configured to allow a user to reposition the external fixation system once attached to a patient.

In Example 19, the external fixation system of Example 18 can optionally include the first distraction handle and the second distraction handle each being pivotably connected to the crossbar.

In Example 20, the external fixation system of any one of or any combination of Examples 15-19 can optionally include a second frame strut, a third pivot housing, and a fourth pivot housing. The second frame strut can fix a second relative position between the proximal ring frame and the distal ring frame. The third pivot housing can pivotably couple the proximal ring frame and the second frame strut. The fourth pivot housing can pivotably couple the distal ring frame and the second frame strut.

### DESCRIPTION OF THE FIGURES

The above-mentioned and other features and advantages of this invention, and the manner of attaining them, will become more apparent and the invention itself will be better understood by reference to the following description of examples of the invention taken in conjunction with the accompanying drawings, wherein:

FIGS. 1A-1C illustrate external fixation systems in accordance with at least one example of the present disclosure.

FIGS. 2A and 2B illustrate a clamping assembly in accordance with at least one example of the present disclosure.

FIGS. 3A-3C illustrate a clamping assembly in accordance with at least one example of the present disclosure.

FIGS. 4A-4C illustrate a clamping assembly attached to a ring frame in accordance with at least one example of the present disclosure.

FIGS. 5A-5D illustrate a distraction handle assembly in accordance with at least one example of the present disclosure.

FIGS. 6A and 6B illustrate a distraction extension arm in accordance with at least one example of the present disclosure.

FIGS. 7A-7H illustrate a pivot housing in accordance with at least one example of the present disclosure.

FIG. 8 illustrates a locking pin in accordance with at least one example of the present disclosure.

FIGS. 9A and 9B illustrate a frame strut in accordance with at least one example of the present disclosure.

FIGS. 10-15 illustrate a pivot housing in accordance with at least one example of the present disclosure.

FIGS. 16A-16D illustrate a pivot housing in accordance with at least one example of the present disclosure.

FIGS. 17A-17C illustrate locking retaining ring in accordance with at least one example of the present disclosure.

FIG. 18 illustrates a housing in accordance with at least one example of the present disclosure.

FIGS. 19A and 19B illustrate a pivot housing in accordance with at least one example of the present disclosure.

FIG. 20 illustrates a clamp assembly in accordance with at least one example of the present disclosure.

FIG. 21 illustrates a cam locking nut assembly in accordance with at least one example of the present disclosure.

FIG. 22 illustrates a clamp assembly in accordance with at least one example of the present disclosure.

FIG. 23 illustrates a cam locking nut assembly in accordance with at least one example of the present disclosure.

FIG. 24 illustrates a universal ring-to-post clamp assembly in accordance with at least one example of the present disclosure.

FIG. 25 illustrates a clamping assembly in accordance with at least one example of the present disclosure.

FIG. 26 illustrates a clamping assembly in accordance with at least one example of the present disclosure.

FIGS. 27 and 28 illustrate a clamping assembly in accordance with at least one example of the present disclosure.

FIG. 29 illustrates a clamping assembly in accordance with at least one example of the present disclosure.

FIGS. 30A-30E illustrate a clamping assembly in accordance with at least one example of the present disclosure.

FIGS. 31A-31C illustrate a cammed aperture in accordance with at least one example of the present disclosure.

FIGS. 32A-32C illustrate a clamp assembly in accordance with at least one example of the present disclosure.

FIG. 33 illustrates a pivot ball in accordance with at least one example of the present disclosure.

FIG. 34 illustrates a clamp assembly in accordance with at least one example of the present disclosure.

FIG. 35 illustrates a clamp assembly in accordance with at least one example of the present disclosure.

FIG. 36 illustrates a clamp assembly in accordance with at least one example of the present disclosure.

FIG. 37 illustrates an elbow clamp assembly in accordance with at least one example of the present disclosure.

FIG. 38 illustrates a shaft clamp assembly in accordance with at least one example of the present disclosure.

FIG. 39 illustrates a clamping assembly in accordance with at least one example of the present disclosure.

FIG. 40 illustrates a pivot housing in accordance with at least one example of the present disclosure.

FIGS. 41A-41F illustrates a clamping assembly in accordance with at least one example of the present disclosure.

FIG. 42 illustrates a clamping assembly in accordance with at least one example of the present disclosure.

FIGS. 43A-43D illustrate a clamping assembly in accordance with at least one example of the present disclosure.

Corresponding reference characters indicate corresponding parts throughout the several views. The exemplifications set out herein illustrate examples of the invention, and such exemplifications are not to be construed as limiting the scope of the invention any manner.

### DETAILED DESCRIPTION

The above-mentioned aspects of the present application and the manner of obtaining them will become more apparent and the teachings of the present application itself will be better understood by reference to the following description of the embodiments of the present application taken in conjunction with the accompanying drawings.

The embodiments of the present application described below are not intended to be exhaustive or to limit the teachings of the present application to the precise forms disclosed in the following detailed description. Rather, the embodiments are chosen and described so that others skilled in the art may appreciate and understand the principles and practices of the present application.

Unless defined otherwise, all technical and scientific terms used herein have the same meaning as commonly understood by one of ordinary skill in the art to which this application belongs. Although any method and materials similar or equivalent to those described herein can be used in the practice or testing of the present application, the specific methods and materials are now described.

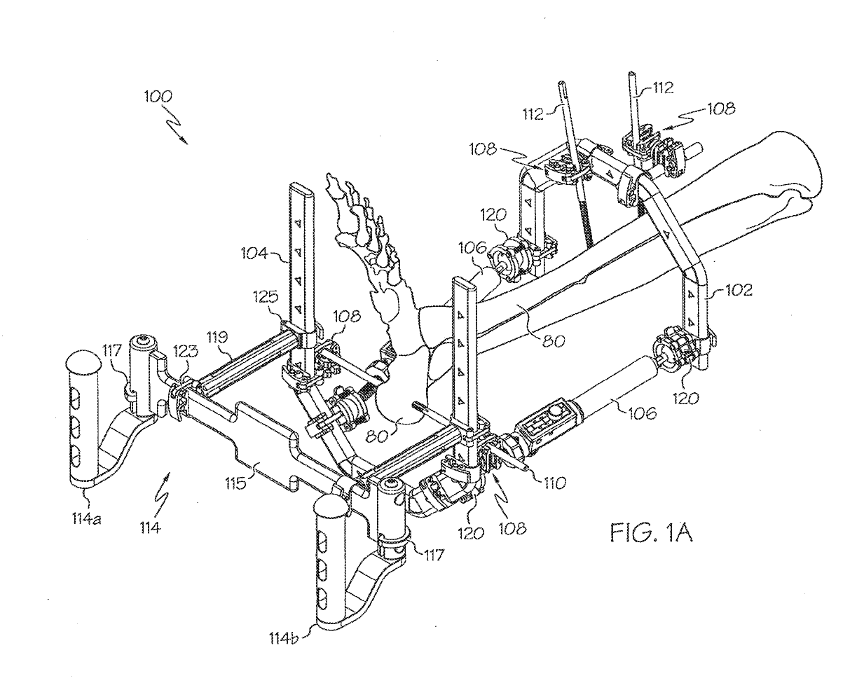

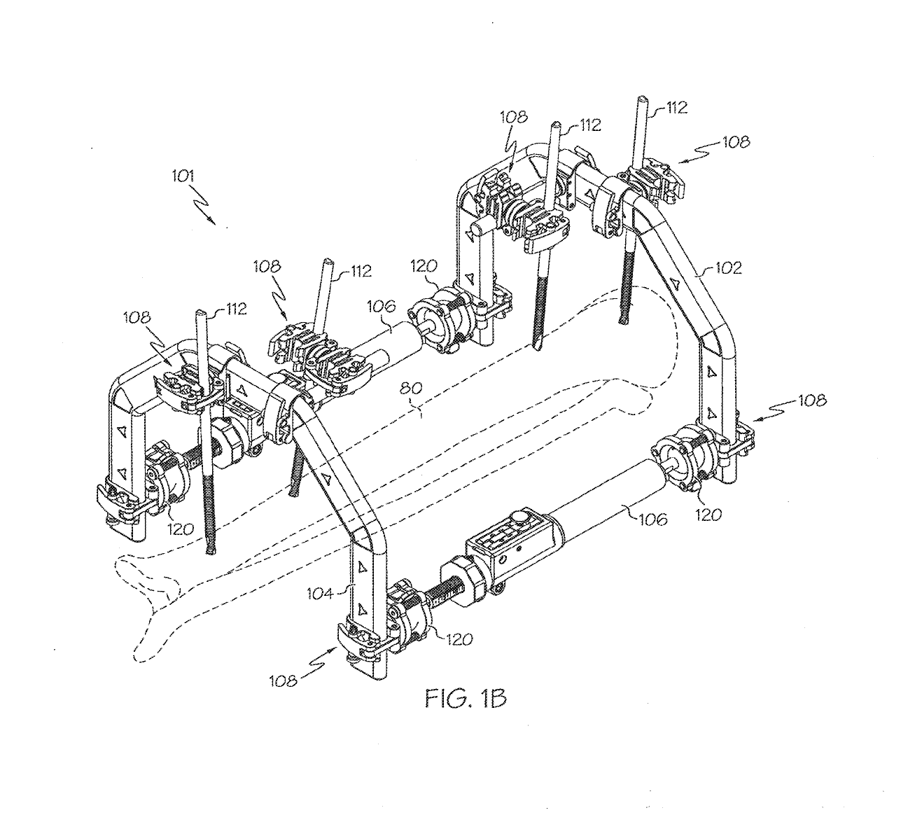

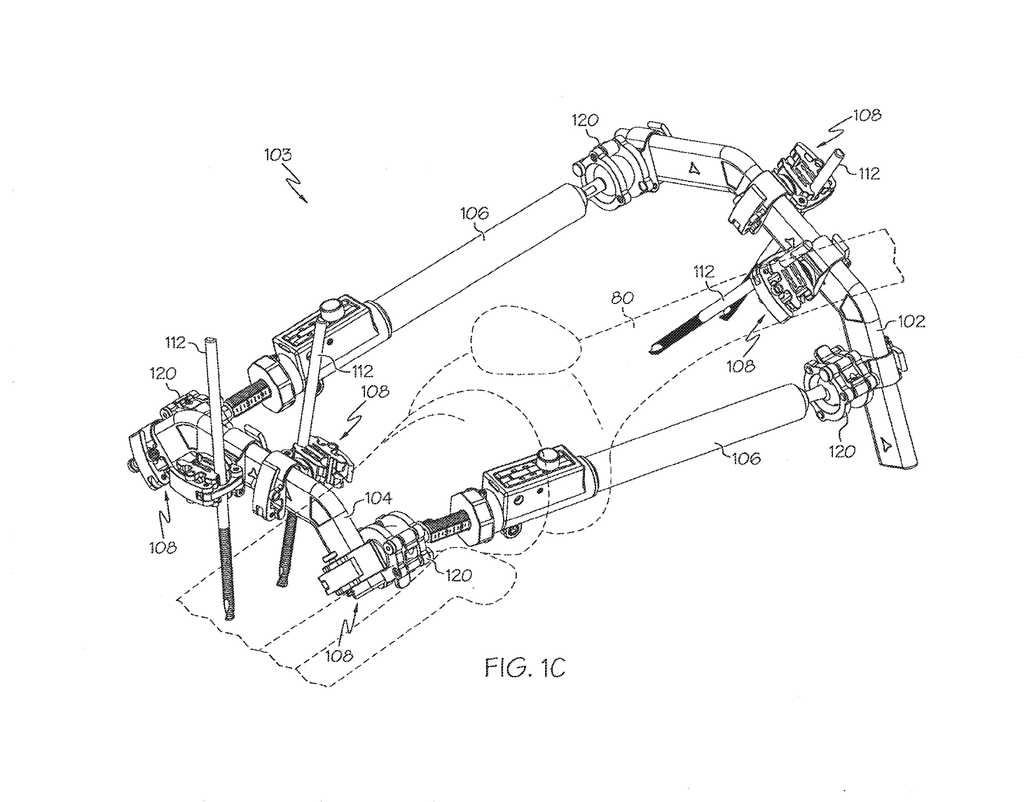

Referring to FIG. 1A, an illustrative ankle spanning external fixation frame system 100 for fixating various bones or bone portions 80 in accordance with the teachings of the present disclosure is illustrated in an environmental view. While this illustrative embodiment depicts the external fixation system 100 associated with a patient's lower extremity, it should be understood and appreciated herein that the teachings of the present disclosure may similarly be utilized on any skeletal portions of the human anatomy, including portions of the human anatomy beyond a patient's lower extremity. Moreover, while the illustrative embodiment depicted in FIG. 1A depicts an ankle spanning external fixation frame, it should be understood and appreciated herein that other lower extremity frame configurations can also be used in accordance with the teachings of the present disclosure. Such other lower extremity frames include, but are not limited to, a mid-shaft tibia spanning frame (such as frame 101 shown in FIG. 1B) and a knee spanning frame (such as frame 103 shown in FIG. 1C).

Referring specifically to FIGS. 1A, 1B and 1C, the external fixation frame systems 100, 101 and 103 each include a proximal ring frame 102 and a distal ring frame 104 positioned on opposite sides of a fracture/fusion site of a patient's bones 80. The proximal and distal ring frames 102, 104 are connected to each other by one or more frame connectors or struts (e.g., ratcheting struts) 106, which can be selected from various sizes and configurations as needed. Illustrative frame connecting struts 106 that may be used in accordance with the teachings of the present disclosure include, but are not limited to, those described in commonly assigned U.S. patent application Ser. No. 13/464,502, which was filed on May 4, 2012, and published as U.S. Patent Publication No. 2013/0296857 on Nov. 7, 2013. The entire disclosure of this application is hereby incorporated in its entirety by this reference.

Various clamps or clamping assemblies 108, which will be described in more detail below, can be used with the frame connectors 106 or independently of the frame connectors 106 for attaching bone pins or wires (e.g., transfixing pins, such as indicated by reference numeral 110), and/or rods, bars, or other fixation devices (e.g., bone screws, such as indicated by reference numeral 112) as desirable for a particular fixation procedure. While not necessarily required herein, the proximal and distal frames 102, 104, the frame connectors 106, the clamping assemblies 108, or any portions thereof, can be radiographically translucent such that the fixation system 100, 101 and 103, when installed, can allow viewing of a fracture/fusion site of the bones 80 on X-ray film. The radiolucent components (or portions thereof) can be formed of, for example, carbon, composite, carbon fiber, or other radiolucent materials.

As will be explained in detail below, various different kinds of clamping assemblies 108 may be used with the external fixation systems 100, 101 and 103 of the present teachings. The type, number, size and order of the various clamping assemblies used to assemble the external fixation frame will not only depend on what type of fixation procedure is being performed, but will also depend on the amount of rotational and translational degrees of freedom desired between the interconnected components. Some of the components that may be interconnected by the clamping assemblies of the present disclosure include, but are not necessarily limited to, proximal and distal ring frames 102, 104, frame connector struts 106, bone screws 112, pins 110, wires, rods, bars, shafts, and other such fixation devices.

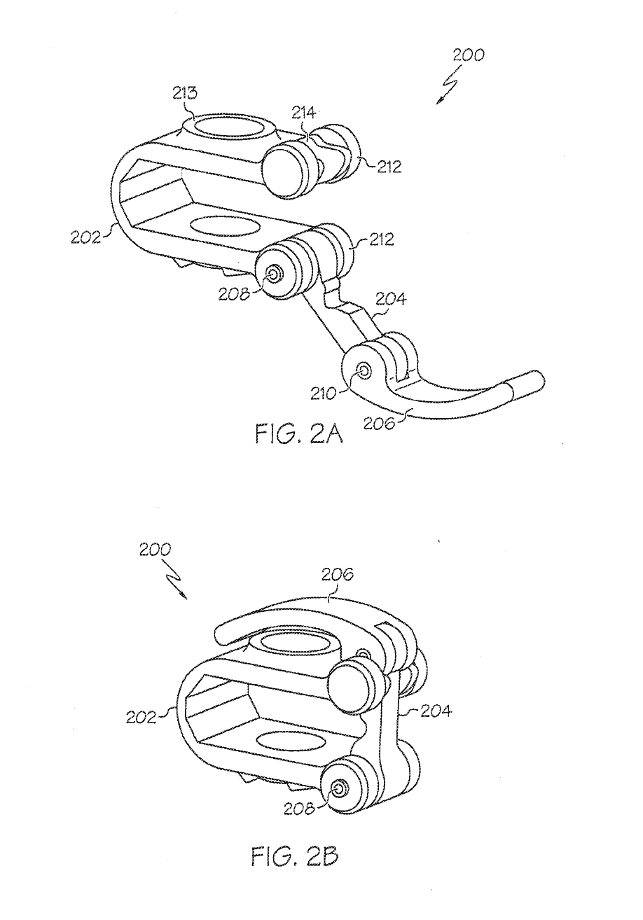

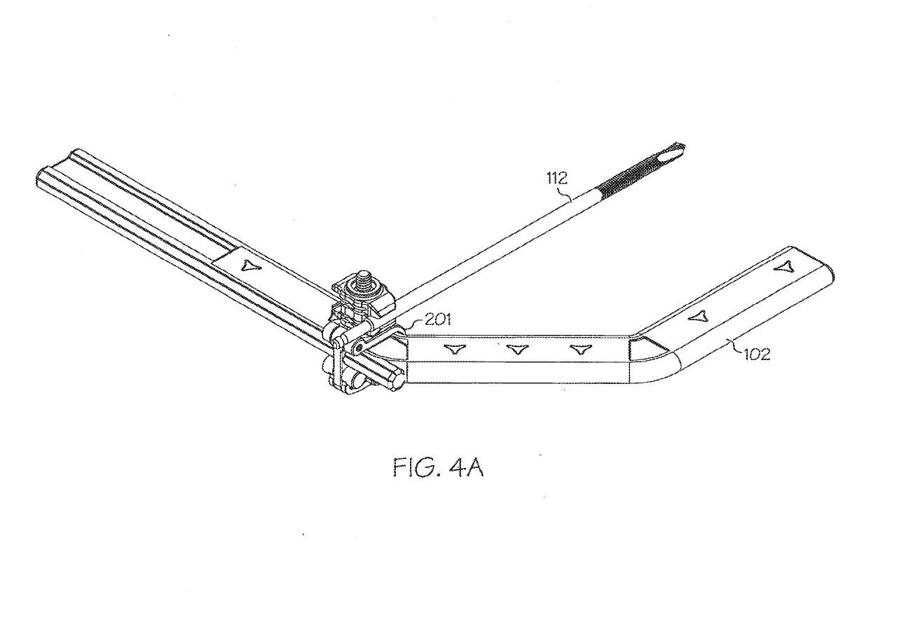

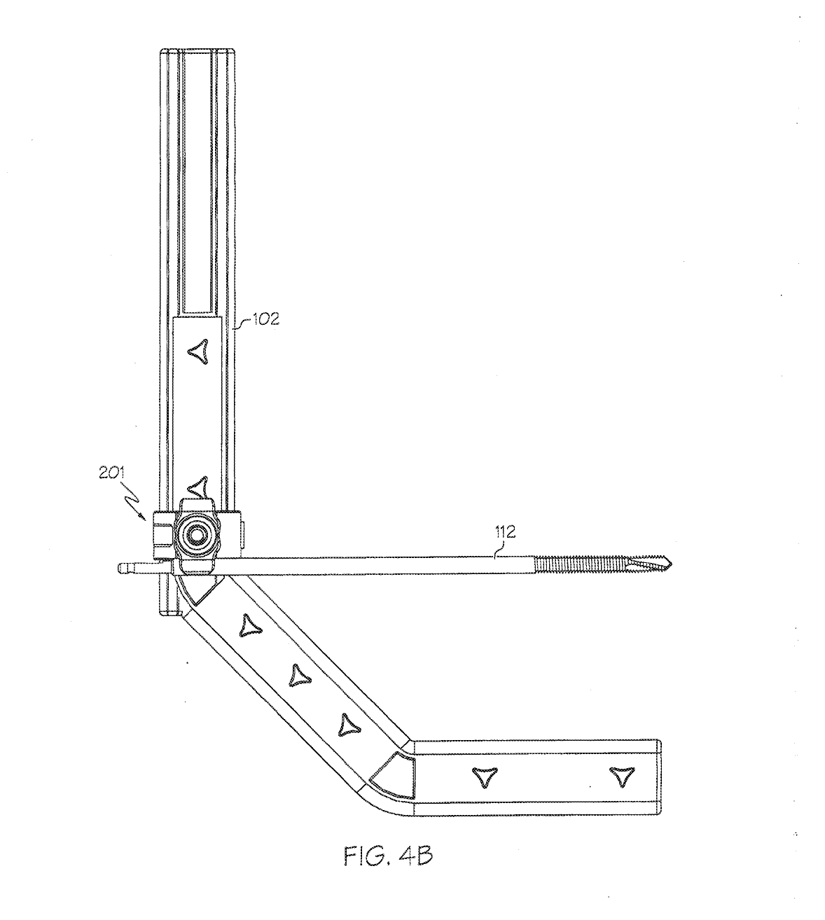

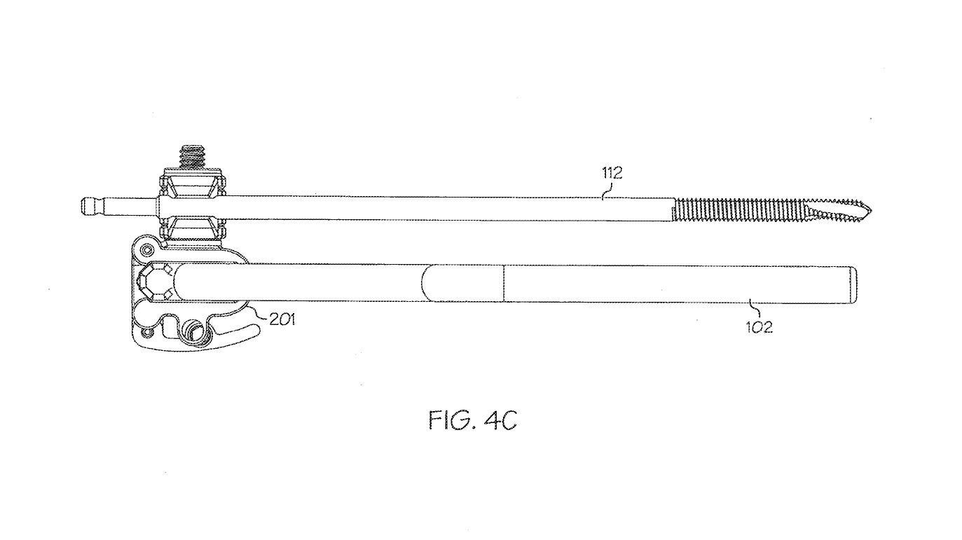

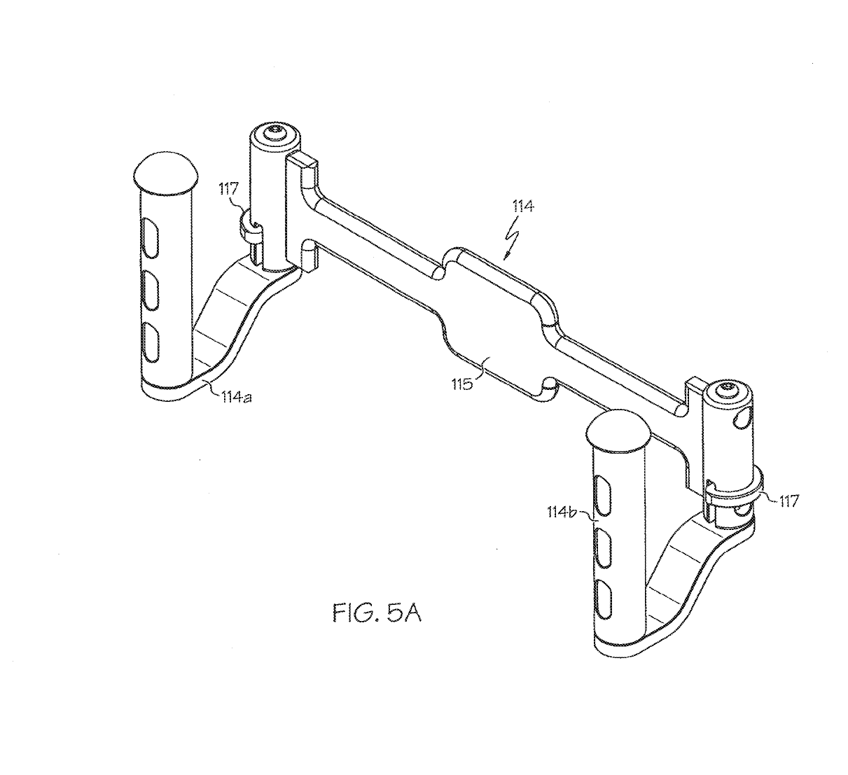

Referring now to various clamping assemblies 108 that may be used in accordance with the present disclosure, FIGS. 2A and 2B depict a first illustrative clamping assembly 200 that includes a clamp body 202, a locking arm 204, a cam arm 206, a locking arm pivot pin 208 and a cam arm pivot pin 210. During assembly of an external fixation frame system 100, 101, 103, the clamping assembly 200 is configured to be snapped onto a ring frame 102, 104 or a rod by positioning the clamp body 202 substantially perpendicular to the ring leg or rod and applying pressure to force end jaws 212 open and over the ring or rod (see FIGS. 4A, 4B and 4C, for instance, which illustrates an illustrative clamping assembly 201 attached to a ring frame 102). Alternatively, the clamping assembly 200 can be snapped onto the ring frame or rod by positioning the clamp body 202 at the end of a ring leg or rod and applying pressure to slide the body onto the ring or rod.

Once positioned on the ring frame 102, 104 or rod, the locking arm 204 is actuatable to compress and center the ring frame or rod inside the clamp body 202. To achieve this, the clamp may be provisionally locked by rotating the locking arm 204 (via the locking arm pivot pin 208) towards and into a cam arm pocket 214. Once the surgeon is satisfied with the position and fixation of the bone fragments 80, the frame or fixator is definitively locked without the use of additional tools or equipment. To achieve the definitive lock, the cam arm 206 is rotated towards the clamp body 202 via the cam arm pivot pin 210 located at the center of the cam arm 206 until it approximates a top surface 213 of the clamp body 202.

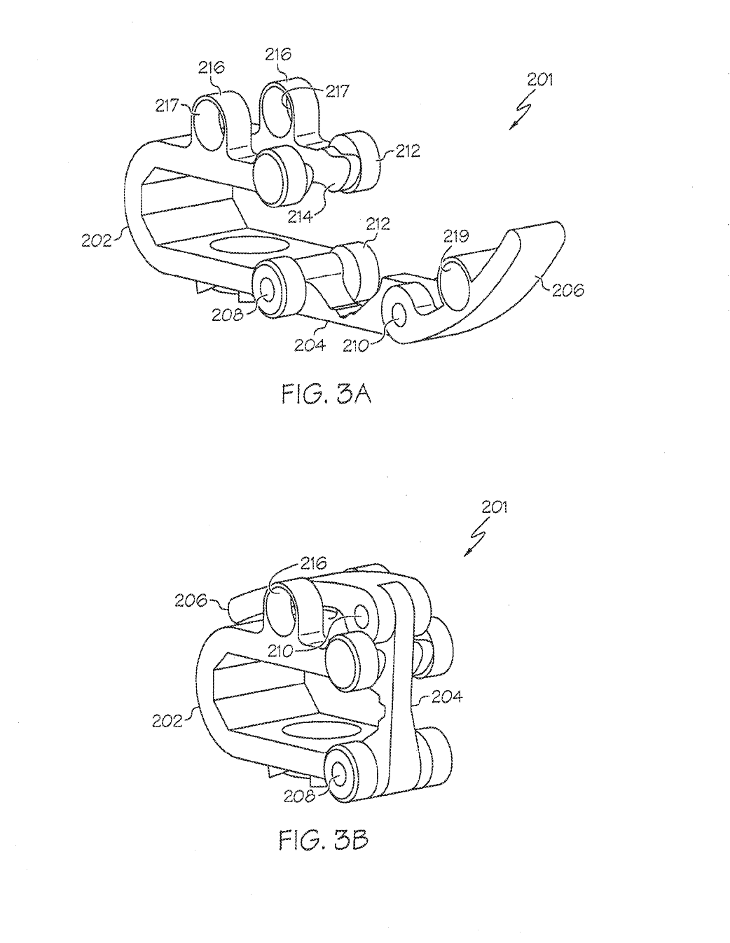

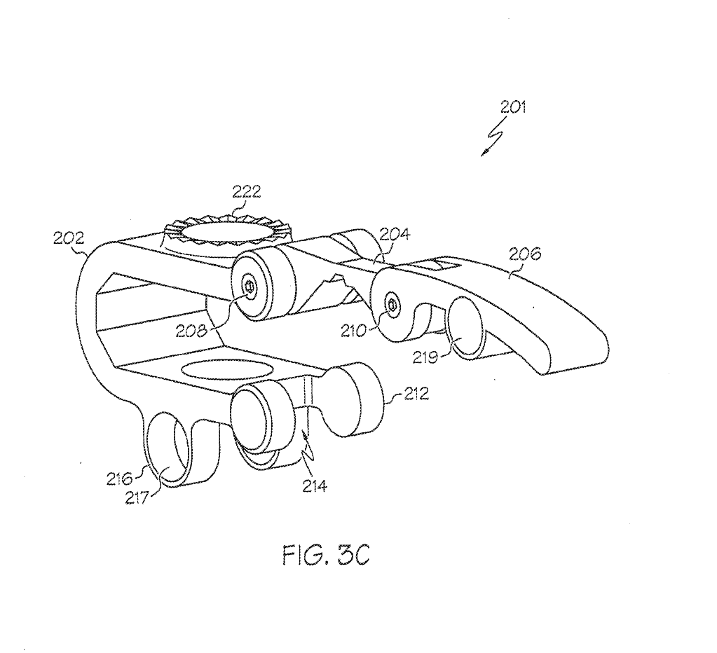

Once the cam arm 206 is positioned against the clamp body 202 during a definitive locking process, in accordance with certain aspects of the present disclosure, the cam arm 206 can be further locked into place by utilizing a locking pin that is configured to be inserted through the cam arm 206. More particularly, FIGS. 3A, 3B and 3C illustrate a clamping assembly 201 having a pair of upwardly projecting tabs 216 extending from the clamp body 202 and each having a through-hole 217 formed therein. When the cam arm 206 is positioned against the clamp body 202, the through-holes 217 align with a through-hole 219 formed into the cam arm 206 such that a common through-hole is created. The locking pin (such as locking pin 144) can then be inserted through this common through-hole, thereby preventing the cam arm 206 from being lifted away from the clamp body 202 until the locking pin 144 is first removed.

By being able to provisionally lock any of the presently disclosed clamping assemblies, the surgeon is not only able to construct the frame or fixator on the patient as presented (i.e., the frame can adapt to the patient's unique anatomy as opposed to requiring the patient's anatomy to first be adjusted in order to fit the frame), but the surgeon can perform the process without needing to utilize additional tools or equipment. As such, the surgeon will be able to position, align and stabilize the bone fragments with the assistance of a C-arm (X-ray) by directly manipulatingFfi the frame itself.

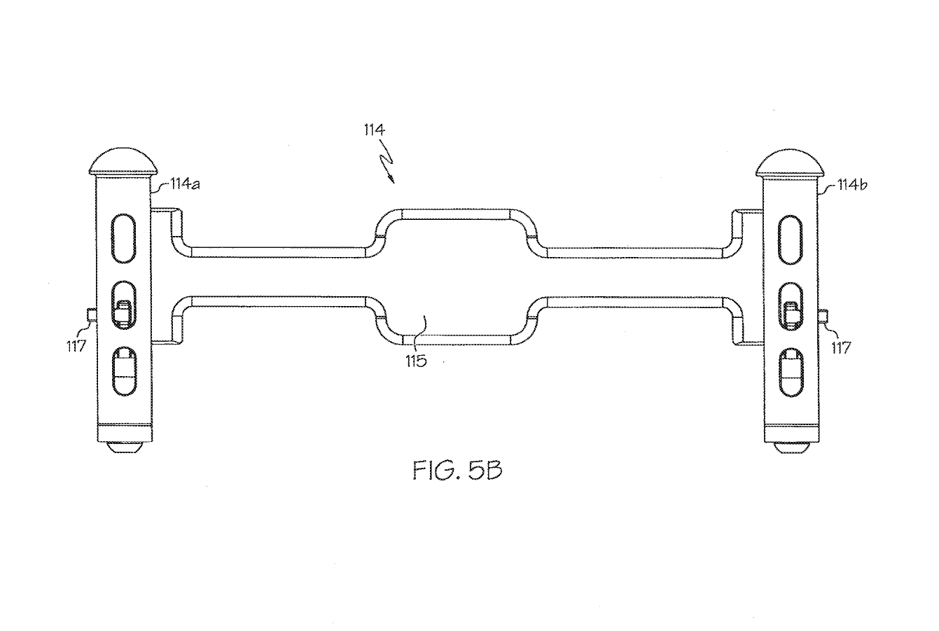

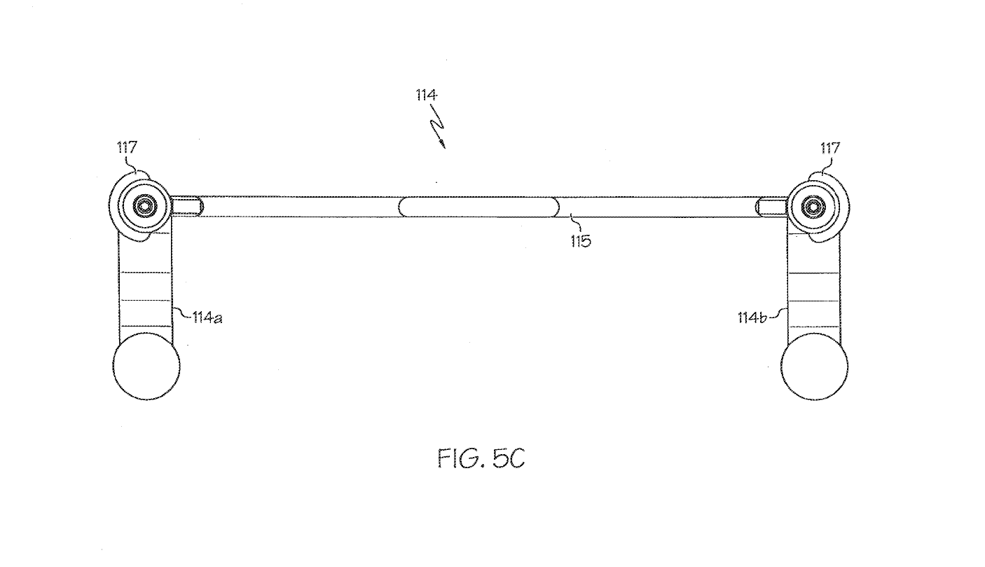

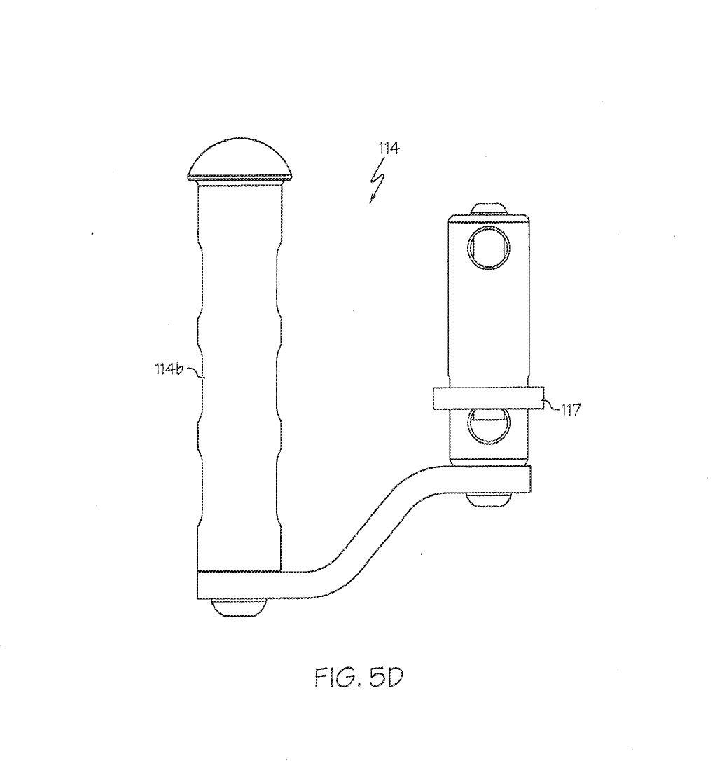

To position, align and stabilize the bone fragments once a clamping assembly (such as clamping assembly 200 or 201, for instance) is provisionally locked to the fixation frame, in accordance with certain aspects of the present disclosure, a distraction handle assembly 114 connected to the distal ring frame 104 can be utilized. In accordance with this aspect of the present disclosure, and specifically referring to FIGS. 5A-5D, the distraction handle assembly 114 includes a pair of distraction handles 114A, 114B that can be manually gripped by the surgeon to adjust the frame during the fracture reduction process as needed. In other words, the surgeon grips the distraction handles 114A, 114B and manipulates (e.g., pulls, pushes, twists) the handles as needed, thereby causing the frame 100, 101 and 103 to respond and change its orientation with respect to the patient's anatomy.

The pair of distraction handles 114A, 114B are connected by a cross bar assembly 115, which provides a gripping feature for the distraction arms. In accordance with certain aspects of the present teachings, the distraction handle assembly 114 may include a handle lock ring 117 that is configured to lock the handle assembly 114 in an open position during its operation. More particularly, in accordance with certain aspects of the present disclosure, the distraction handle assembly 114 is capable of being adjusted between two position—i.e., a first position (open state) where the distraction handles 114A, 114B are at 90° to the cross bar 115 and a second position (closed state) where the handles are collapsed against the cross bar. To rotate the arms from the open state to the closed state, the user pulls up on each of the lock rings 117 located around the handle pivot and rotates the handle assembly towards the cross bar.

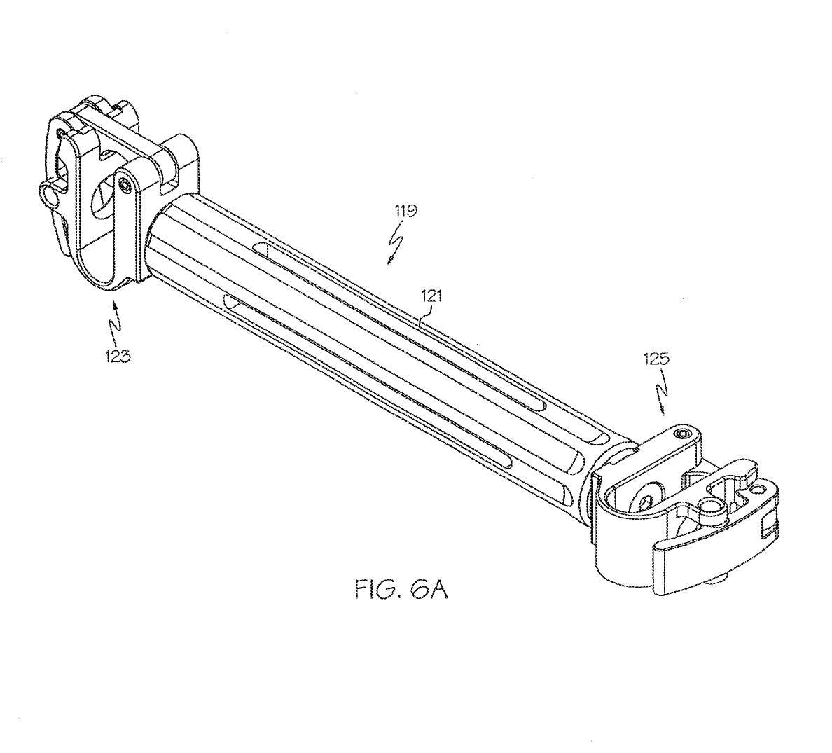

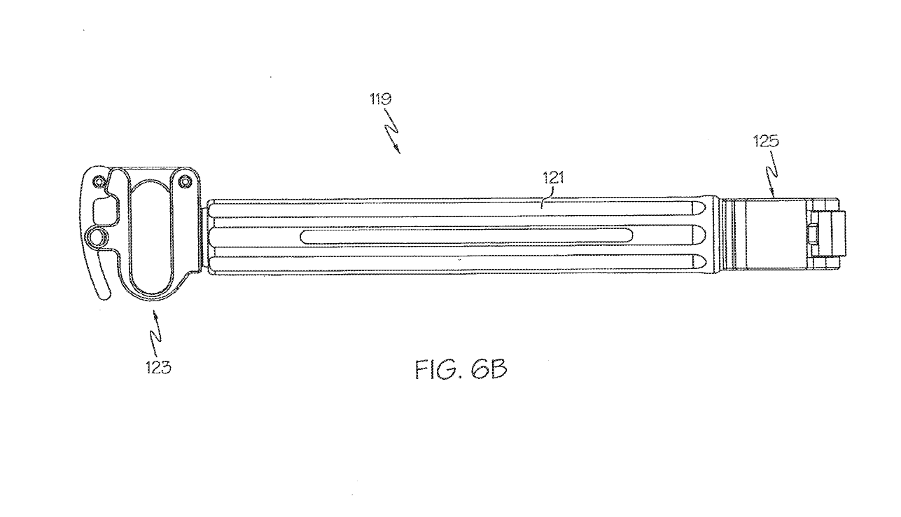

In addition, and to ensure that the surgeon is able to safely adjust the external fixation assembly frames 100, 101 and 103 without exposing his or her hands to the X-ray field during the adjustment process, the handles 114A, 114B of the distraction handle assembly 114 are extended from the distal ring frame 104 via a pair of distraction extension arms 119 (see FIGS. 1A, 6A and 6B). According to certain aspects herein, each extension arm 119 has a barrel 121 that provides a connection between the cross bar assembly 115 and the distal ring frame 104. A fixed ring/cross bar clamp 123 connects to the ring 104 or cross bar 115 in a fixed position, while a pivoting ring/cross bar clamp 125 connects the ring 104 or cross bar 115 in such a manner that it is rotatable 360°. As those of skill in the art will understand and appreciate herein, the basic concept for the use of the distraction handle assembly 114 is to connect the assembly to the pre-constructed external fixation frame (i.e., frames 100, 101 and 103) via the use of two distraction arm extension assemblies 119. The distraction handle assembly is configured to be used by a surgeon as an instrument to assist in the distraction and alignment of bone fractures which are “provisionally” stabilized by the frame, as well as to protect or reduce the intensity of exposure to imaging radiation by allowing the surgeon to position his or her hands away from the frame during a radiological event.

Unlike traditional external fixation systems that require surgeons to lock and unlock various portions of the frame in order to manually adjust and manipulate the orientation of the fixation frame with respect to a patient's anatomy, the external fixation frame systems 100, 101 and 103 of the present disclosure are capable of being polyaxially adjusted (e.g., adjustment along several different axes) through a range of motion having a 360-degree pattern from a longitudinal axis (without the use of additional tools or equipment) while the frame is provisionally locked into place. To achieve such polyaxial adjustment, an adjustable pivot housing 120 is incorporated into the external fixation frame system 100, 101 and 103 and is applied to the patient's bones prior to reducing the fracture. As used herein, the term “polyaxial” or “polyaxially” refers to the ability of a first element to pivot in multiple planes with respect to a second element to which the first element is coupled. The adjustable locking polyaxial pivot housing 120 can be used with either a bilateral frame system or a unilateral frame system. When incorporated into the external fixation frame system, it allows the surgeon to provisionally position the bone fragments via C-arm (X-ray) to align, stabilize and prevent additional neurovascular damage without the need to definitively lock the frame by means of additional instruments or actions.

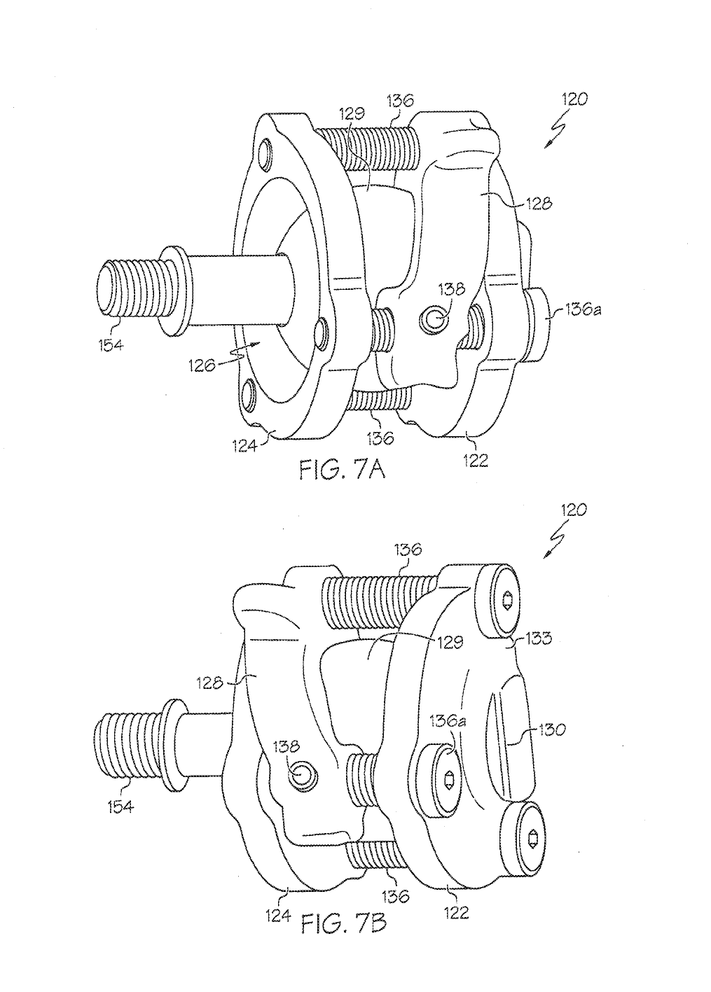

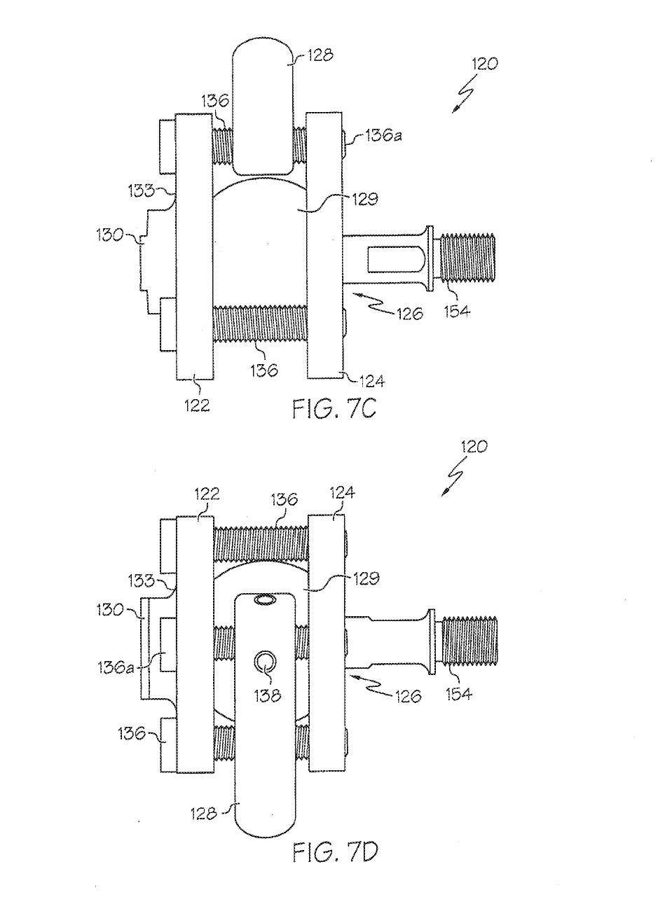

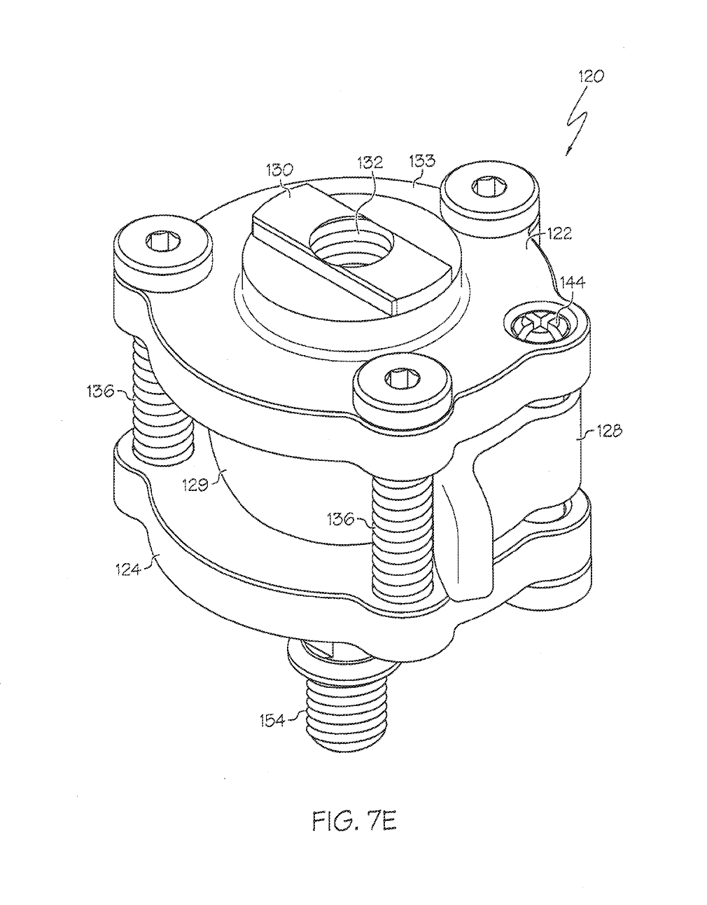

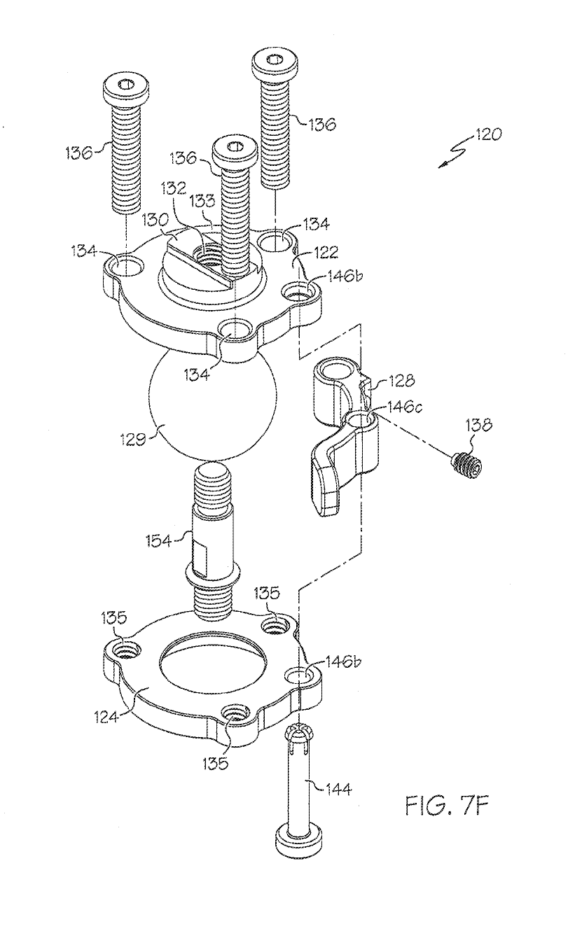

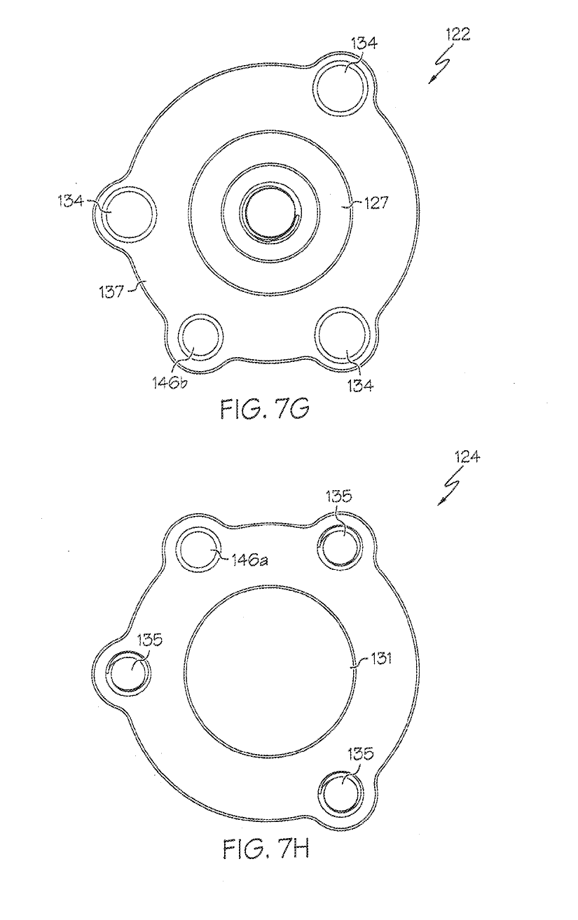

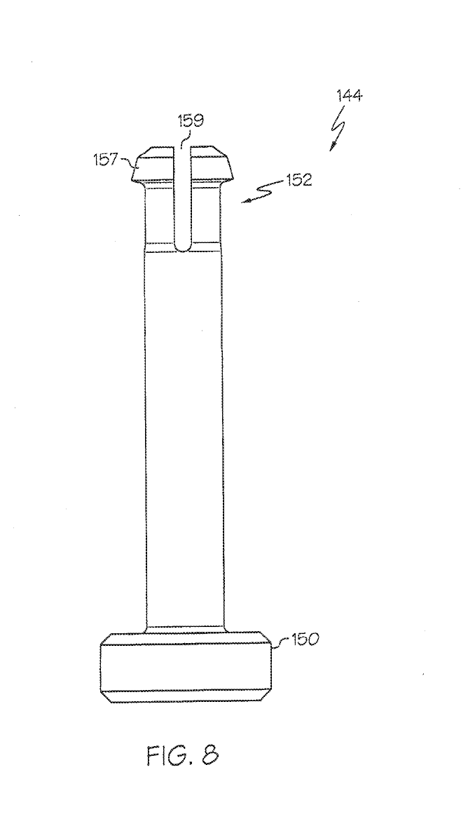

Various views of a polyaxial pivot housing in accordance with one illustrative embodiment of the present disclosure is shown in FIGS. 7A-7H. In accordance with this illustrative embodiment, the polyaxial pivot housing 120 includes a top ring 122, a bottom ring 124, a sphere/post assembly 126 and a locking arm 128. The top ring 122 has a platform 130 extending from its top surface 133 that is configured to connect to a clamping assembly (clamping assembly 200 or 201, for example) by threading the clamp into a threaded bore hole 132 of the platform 130. When the housing 120 is assembled, the top ring 122 provides a compressive loading along with the bottom ring 124 to thereby hold and center the sphere/post assembly's spherical body 129 therebetween. As shown in FIG. 7G, to hold and center the spherical body 129 in response to the compressive loading of the top ring 122, the bottom surface 137 of the top ring 122 includes a substantially circular recessed cavity 127 that is dimensioned to accept the top portion of the spherical body 129 as the pivot housing 120 is locked relative to the frame. Similarly, as shown in FIG. 7H, the bottom ring 124 includes a substantially circular cavity 131 that is dimensioned to accept the bottom portion of the spherical body 129 as the housing is locked into place.

The top ring 122 also includes three clearance holes 134 adapted to receive locking bolts 136 that pass through the top ring 122 and thread into the bottom ring 124 via three corresponding threaded holes 135. Because of the threaded relationship between the locking bolts 136 and the bottom ring 124, when the frame 100, 101 and 103 is initially adjusted by the surgeon by pulling upon the distraction handle assembly 114, the locking bolts 136 allow the polyaxial pivot housing 120 to be provisionally locked into place in response to the compressive forces and tension applied to the spherical body 129 by the top and bottom rings 122, 124 (i.e., it achieves a friction fit). Specifically, the provisional lock is obtained when the top and bottom rings 122, 124 are positioned so that the compressive force is applied to the spherical body 129 to prevent movement by soft-tissue forces. The friction fit and soft tissue tension allows the surgeon to provisionally lock the frame and make minor adjustments to the bone alignment. Once the surgeon is satisfied with the fixation of the bone fragments, the frame can then be definitively locked to maintain the bone fragments within the desired alignment.

To definitively lock the housing 120, in accordance with certain embodiments herein one of the locking bolts 136A is connected to the locking arm 128 by way of a blind set screw 138 that passes through a threaded hole (not shown) of the locking arm 128 and presses against the locking bolt 136A. As the locking cam arm 128 is rotated in a clockwise direction, the set screw 138 prevents the locking bolt from moving independent of the locking arm 128. As a result, as the locking arm 128 is rotated about a longitudinal axis upon being pulled directionally towards the spherical body 129, the bottom ring 124 is caused to move towards the top ring 122, thereby compressing the spherical body 129 between the top and bottom rings 122, 124. This clockwise rotation of the locking arm 128 causes the attached set screw 138 to also turn, thereby compressing the top and bottom rings 122, 124 further against the spherical body 129 to create a “locking fit” that prevents the sphere/post assembly 126 from moving with respect to the fixation frame 100, 101 and 103.

As shown in FIGS. 7E and 8, in accordance with certain aspects of the present disclosure, the pivot housing 120 may further include a locking pin 144 that is configured to hold the locking arm 128 in place once it has been definitively locked. According to this embodiment, the top and bottom rings 122, 124, together with the locking arm 128, include a series of aligned through-holes that are configured to receive the locking pin 144 as it is inserted therethrough. Specifically, the locking pin 144 is inserted into a through-hole 146A of the bottom ring 124 from its outer surface, and particularly in such a manner that the locking pin 144 continues to pass through the through-hole of the locking arm 146c and finally into the through-hole 146B of the top ring 122. As shown in FIG. 8, the locking pin 144 includes a head portion 150 on one end and a locking mechanism 152 on its opposite end. The head portion 150 is dimensioned in such a manner that it is unable to enter the through-hole 146A of the bottom ring 124 as the locking pin is inserted therein. Since the head portion 150 has a circumferential dimension larger than that of the through-hole 146A, when the locking pin 124 is inserted therethrough, the head portion 150 is configured to serve as a stop surface as it flushingly engages the outer surface of the bottom ring 124. The locking mechanism 152 of the locking pin 124 is in turn configured to create a locking engagement with the through-hole 146B of the top ring 122 when the locking pin 124 is fully inserted therein. While various different locking arrangements may be utilized in accordance with the present disclosure, in accordance with certain illustrative aspects herein, the locking mechanism 152 may be shaped in such a manner that it creates a latching engagement with the through-hole. For instance, the locking pin 144 and/or its locking mechanism 152 may be manufactured from a material that is compressible or elastically deformable (e.g., plastic, rubber). In accordance with this illustrative aspect, the locking mechanism 152 may include a slightly flared portion 157 that is configured to compress as it passes through the through-hole 146B, yet expand back to its original dimensions after passing fully therethrough. To accomplish this expansion/retraction, a slot 159 may be formed into the locking mechanism 152 that will allow the flared portion 157 to change its circumferential dimensions by allowing it to move between an expanded and retracted position. Once returning to its original dimensions, the locking mechanism 152 will expand to a size that is slightly larger than the dimensions of the through-hole 146B, thereby locking it into place relative thereto.

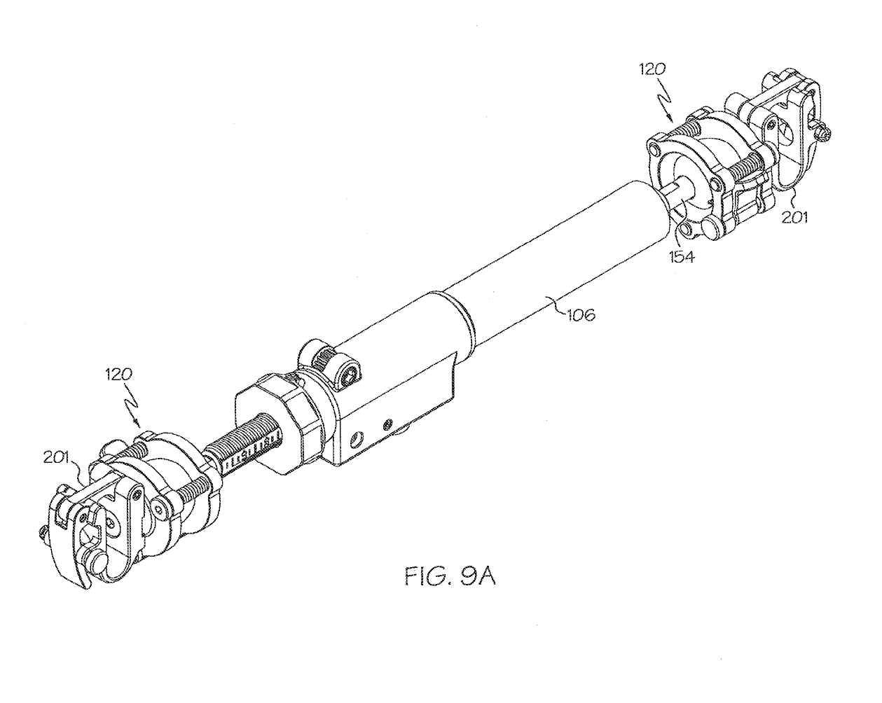

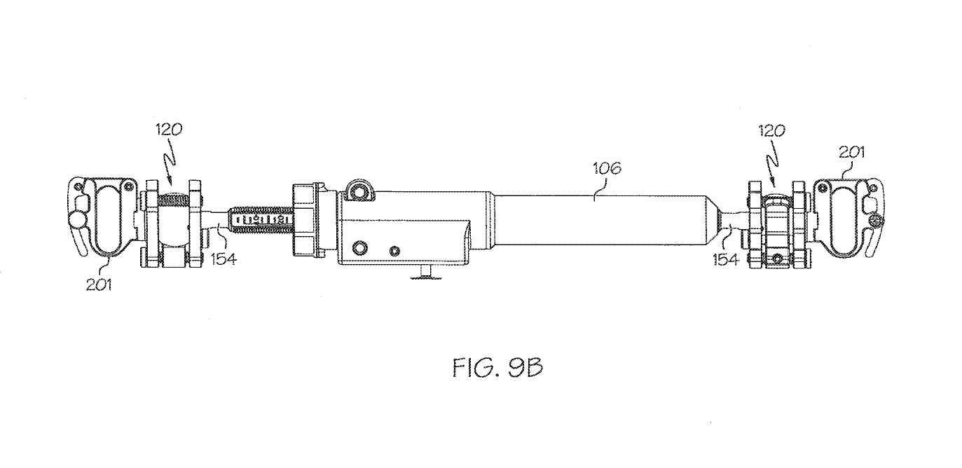

Extending from the spherical body 129 is an attachment post 154 that is configured to attach the pivot housing 120 to a frame connector or ratcheting strut 106 that bridges the proximal ring frame 102 to the distal ring frame 104 (see FIGS. 9A and 9B, for instance). While the attachment post 154 and the spherical body 129 can be manufactured as a single component or unit (e.g., overmolded into a single unit), in accordance with certain aspects of the present disclosure, the attachment post 154 and the spherical body 129 may be independent of one another. For instance, the spherical body 129 may include a threaded aperture (not shown) that is configured to receive a threaded portion (not shown) of the attachment post 154.

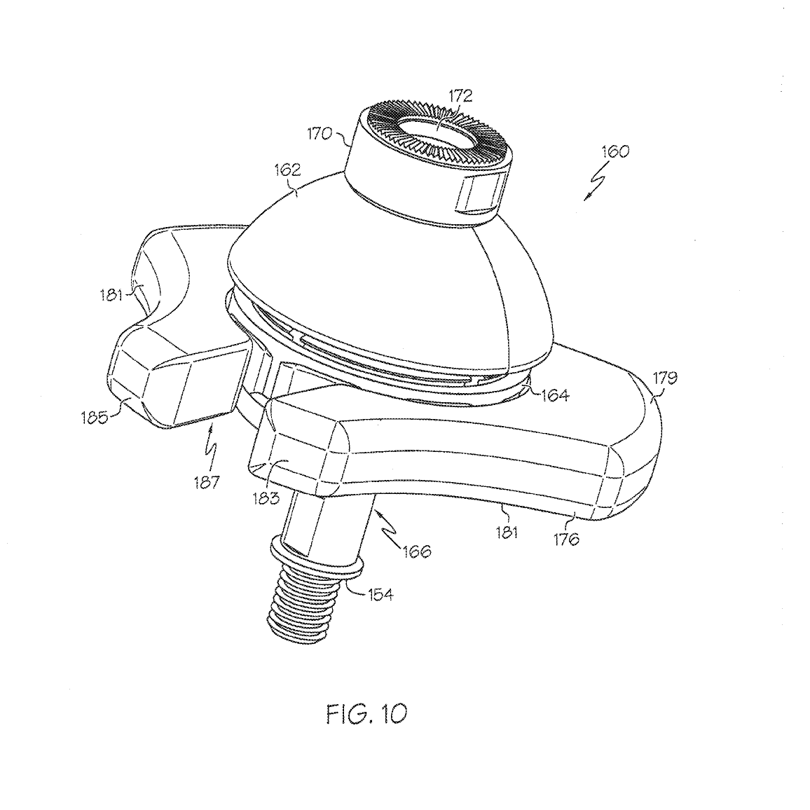

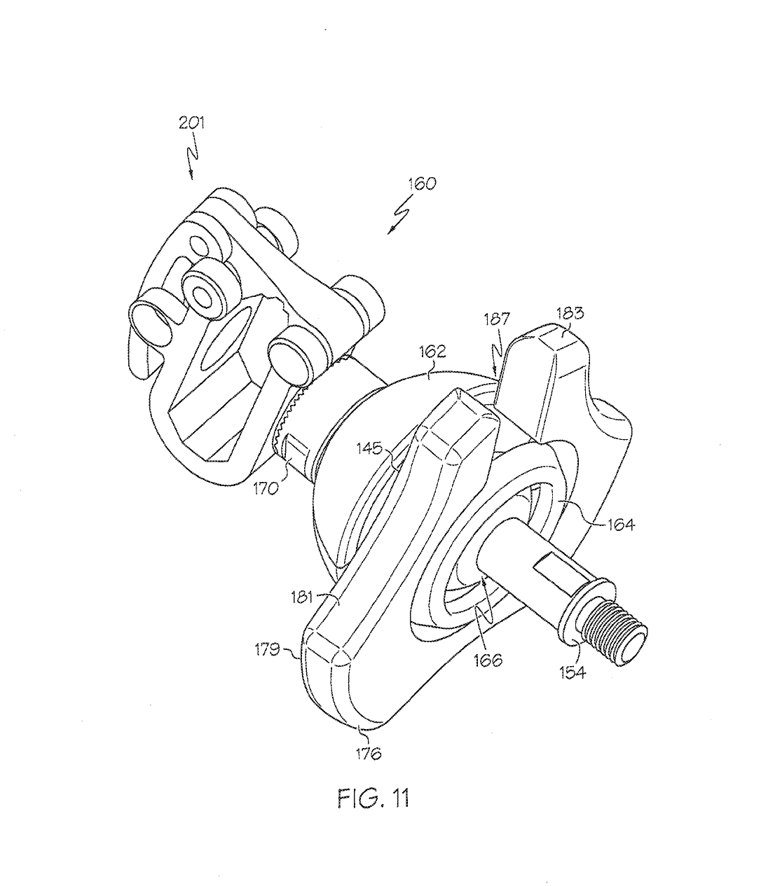

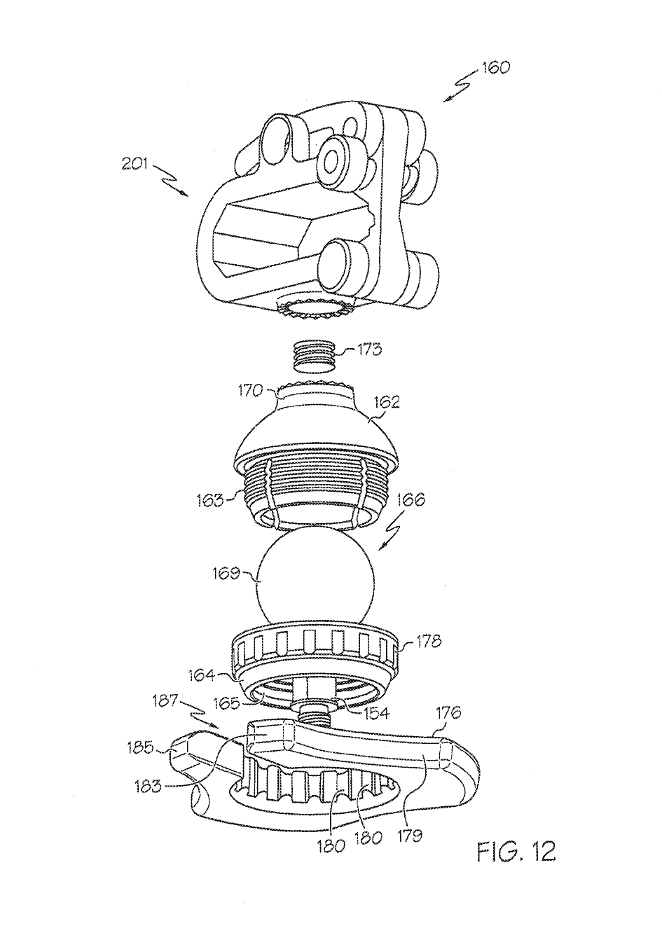

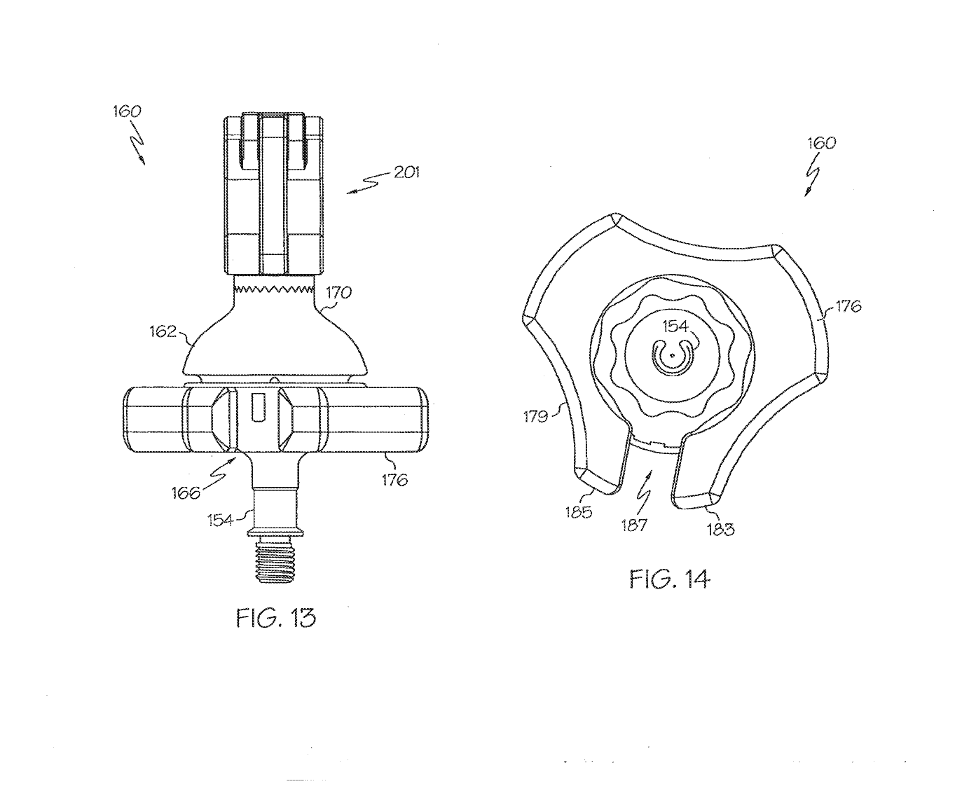

As shown in FIGS. 10-14, another polyaxial pivot housing in accordance with the teachings of the present disclosure is illustrated. According to this embodiment, the polyaxial pivot housing 160 includes an outer collet housing 162, a compression collar or locking nut 164 and a sphere/post assembly 166. The outer collet housing 162 has a platform 170 extending from its top surface that is configured to connect to a clamping assembly (such as clamping assembly 201, for instance) by threading the clamp into a threaded bore hole 172 of the platform 170. While the platform 170 may be integral with the outer collet housing 162 such that the two are formed as a single unit, those of skill in the art will understand and appreciate herein that the platform 170 can also be manufactured as a separate piece that is in turn coupled to the housing (e.g., threaded, fused, welded, etc.).

In accordance with certain aspects of the present disclosure, the threaded bore hole 172 is configured to house a spring mechanism 173 (e.g., a coil spring), such that when the housing is assembled to an external fixation frame 100, 101, 103, the spring 173, together with soft tissue tension, allows the surgeon to provisionally lock the external fixation frame and make minor adjustments to the bone alignment as desired.

When the pivot housing 160 is assembled, the outer collet housing 162 provides a compressive loading along with the locking nut 164 to thereby hold and center the spherical body 169 of the sphere/post assembly 166 therebetween. To accomplish this, the outer collet housing 162 includes a series of threads 163 that are configured to threadingly mesh with corresponding threads 165 of the locking nut 164. In accordance with certain alternative aspects of the present disclosure, and as specifically shown in FIG. 15, a pivot housing 161 may also include a separate compression collet 167 that is configured to fit inside of an inner periphery 171 of the outer collet housing 162 such that when the threads 175, 177 of the outer collect housing 162 and the locking nut 164 are engaged, the compression collet 167 compresses around the spherical body 169 to provisionally or definitively lock the pivot housing 160 relative to the fixation frame 100, 101, 103. In accordance with this aspect of the present disclosure, the outer collet housing 162 is configured to prevent the compression collet 167 from rotating and guides the compression collet to compress against the spherical body 169 during the locking process.

To provisionally lock the external fixation frame 100, 101, 103, the threaded compression collar or locking nut 164 presses against the outer collet housing 162 (and/or compression collet 167 if optionally used) to provide a friction fit. Specifically, the provisional lock is obtained when the locking nut 164 and outer collet housing 162 (and/or compression collet 167) are caused to provide a compressive force to the spherical body 169, thus preventing movement by soft-tissue forces. The friction fit and soft tissue tension allows the surgeon to provisionally lock the frame and make minor adjustments to the bone alignment. Once the surgeon is satisfied with the fixation of the bone fragments, the frame can then be definitively locked to maintain the bone fragments within the desired alignment.

To definitively lock the pivot housing 160, the threads 165 of the locking nut 164 are threadingly mated with the threads 163 of the outer collet housing 162, thereby compressing the sphere/post assembly 166 therebetween (or by threadingly mating threads 175 and 177 if the compression collet 167 is optionally used as shown with pivot housing 161). A removable nut socket or wrench 176 can also be removably attached to the outer periphery of the locking nut 164 and used to further tighten the threaded relationship between the outer collet housing 162 and the locking nut 164 if desired. To accomplish this, the outer periphery of the locking nut 164 may contain a series of ridges or notches 178 that are configured to matingly correspond to a series of ridges or notches 180 on the inner periphery of the removable nut wrench 176.

As those of skill in the art will understand herein, the removable nut wrench 176 can have an inner peripheral circumference that is slightly smaller than that of the outer peripheral circumference of the locking nut 164. In accordance with this embodiment, the removable nut wrench 176 is defined by a partially cylindrical knob body 179 having one or more hollowed out or concave portions or flutes 181 that are fabricated into its outer periphery to provide an improved gripping surface (i.e., the surface can have an ergonomic contour that fits the user's hand comfortably). The knob body 179 is further defined by two end portions or arms 183, 185 that are positioned substantially adjacent to one another, yet do not touch each other (i.e., they are separated by a gap 187). As the nut wrench 176 is mated with the locking nut 164, the arms 183, 185 are caused to separate (move away) from each other, thereby increasing the size of the gap 187. While those of skill in the art will understand and appreciate herein that forming a gap or a voided region within an otherwise solid object will impart some inherent elasticity to the object, in accordance with certain aspects of the present disclosure, the body 179 may further be formed of a material having elastic deformation properties to further enhance this quality as desired. By causing the removable nut wrench 176 to be elastically deformable, as the wrench 176 is fitted over the locking nut 164, the wrench will return to its original shape once positioned over the locking nut 164, thereby creating a snapping engagement thereto. After the nut wrench 176 is used to further tighten the locking nut 164, it can then be removed from the housing 160, 161 if desired so that the patient (to which the external fixation frame is installed) is unable to tamper with the frame.

Extending from the spherical body 169 is an attachment post 154 that is configured to connect (e.g., thread) the pivot housing 160, 161 to a frame connector or ratcheting strut 106 that bridges the proximal ring frame 102 to the distal ring frame 104. While the attachment post 154 and the spherical body 169 can be manufactured as a single component or unit, in accordance with certain aspects of the present disclosure, the attachment post 154 and the spherical body 169 may be fabricated independently of one another. For instance, the spherical body 169 may include a threaded aperture that is configured to receive a threaded portion of the attachment post 154. Moreover, the outer collet housing 162 and the compression collet 167 (if used) each may be manufactured from single pieces or multiple pieces interconnected if desired. As such, the present disclosure is not intended to be limited herein.

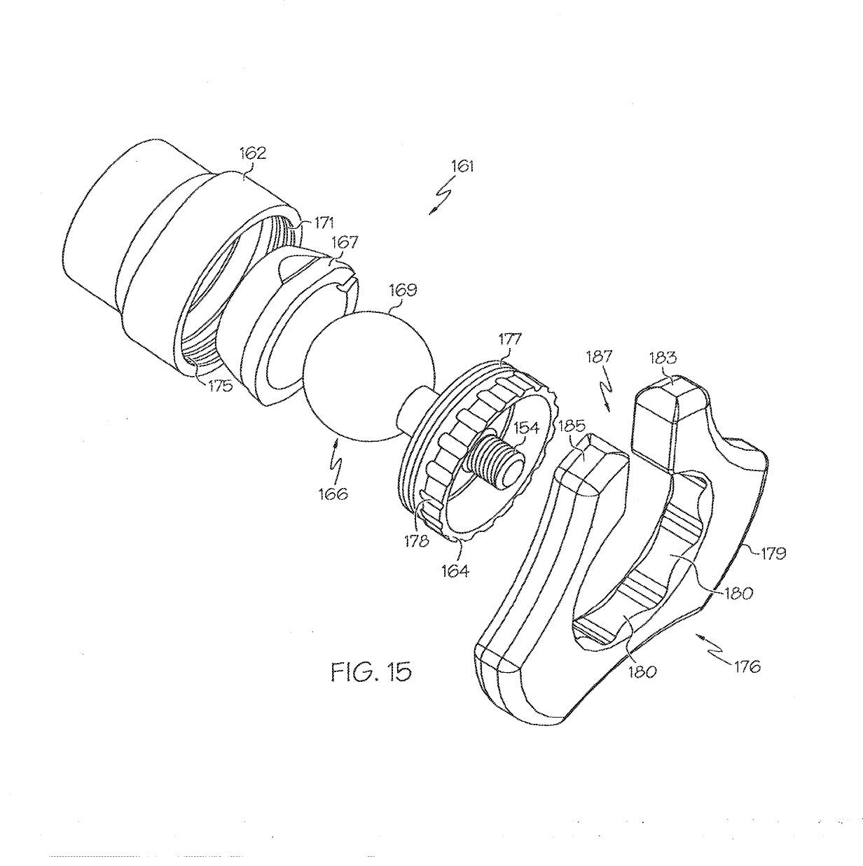

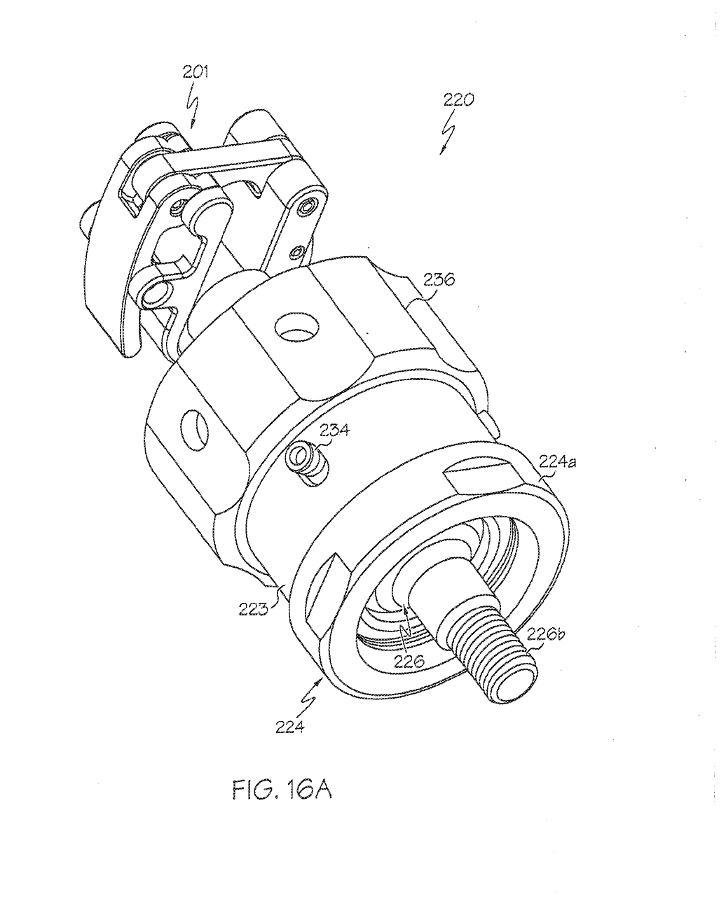

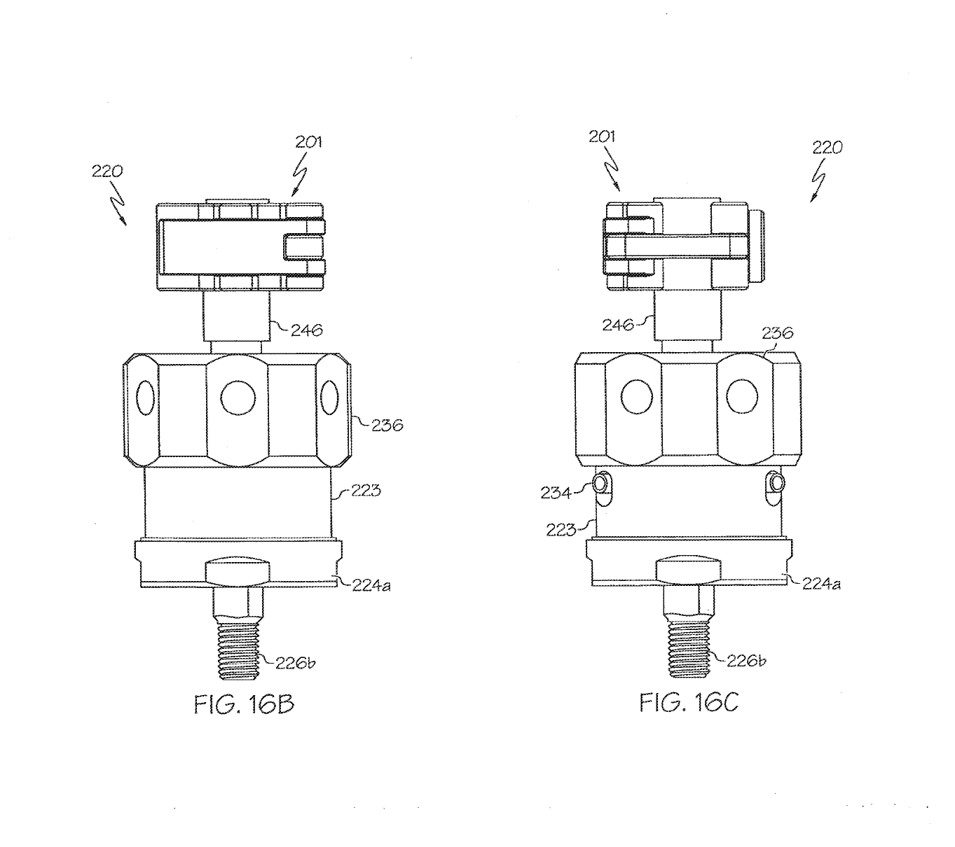

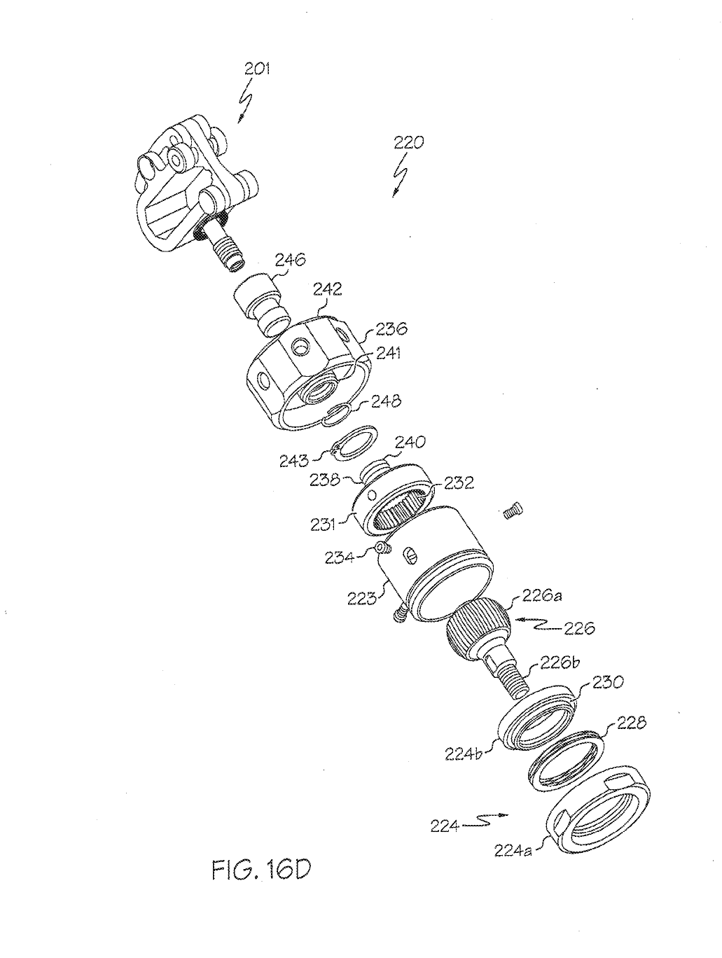

As shown in FIGS. 16A-16D, another illustrative design for a polyaxial pivot housing 220 in accordance with another aspect of the present disclosure is shown. In accordance with this embodiment, the housing 220 includes an outer sleeve 223 that is configured to receive a base assembly 224 and a serrated sphere/post assembly 226, the serrated sphere/post assembly 226 being comprised of a spherical ball portion 226A and a strut attachment post portion 226B. The base assembly 224 is composed of two separate base portions (i.e., a first base portion 224A and a second base portion 224B) that are separated from each other by a wave spring 228 (i.e., the first and second base portions 224A, 224B sandwich the wave spring 228 therebetween). Specifically, the wave spring 228 is configured to be housed within a ridge or shelf 230 of the second base portion 224B. When the housing is assembled, the first and second base portions 224A, 224B mate with each other (via the wave spring 228) and force the serrated sphere/post assembly 226 into the outer sleeve 223. The spherical ball portion 226A of the serrated sphere/post assembly 226 in turn interfaces with a serrated base assembly 231 that has an inner serrated periphery 232 that is configured to matingly receive the outer surface of the spherical ball portion 226A. When the housing 220 is assembled, anti-rotation screws 234 can be inserted into outer sleeve 223 to prevent the serrated base assembly 231 from rotating inside of the outer sleeve 223.

A locking nut portion 236 is in turn configured to be threaded to the serrated base assembly 231 by way of a threaded post 238 that extends from the serrated base assembly 231. In accordance with this aspect of the present disclosure, when the pivot housing 220 is assembled, the serrated base assembly 231 provides a compressive loading along with the locking nut portion 236 to thereby hold and center the serrated spherical ball portion 226A of the sphere/post assembly 226 therebetween. To accomplish this, the threaded post 238 includes a series of threads 240 that are configured to threadingly mesh with corresponding threads 241 within a bore hole 242 of the locking nut portion 236. A retaining ring 243 is held between the top surface of the serrated base assembly 231 and the bottom surface of the locking nut portion 236.

A clamping assembly platform 246 is in turn coupled to the locking nut's bore hole 242 on the side opposite the threaded portion 241. The clamping assembly platform 246 includes a threaded internal aperture (not shown) that is configured to receive a clamping assembly (such as assembly 108). To secure the clamping assembly platform 246 to the locking nut portion 236, a retaining ring 248 is utilized.

As mentioned previously, once the external fixation frame is attached to a patient via percutaneous bone pins, the surgeon will position the bone fragments via C-arm (X-ray) to align, stabilize and prevent additional neurovascular damage. The internal wave spring 228 and soft tissue tension experienced by the presently disclosed pivot housing 220 allows the surgeon to provisionally lock the frame and make minor adjustments to the bone alignment. Once the surgeon is satisfied with the fixation of the bone fragments, the frame may be definitively locked to maintain the bone fragment's alignment. To achieve the definitive lock, the locking nut portion 236 is rotated such that the internal thread moves the serrated base assembly 231 against the serrated surface of the serrated spherical ball portion 226A of the sphere/post assembly 226.

The attachment post portion 226B that extends from the serrated spherical ball is configured to function as an means to connect (e.g., thread) the pivot housing 220 to a frame connector or ratcheting strut 106 that bridges the proximal ring frame 102 to the distal ring frame 104.

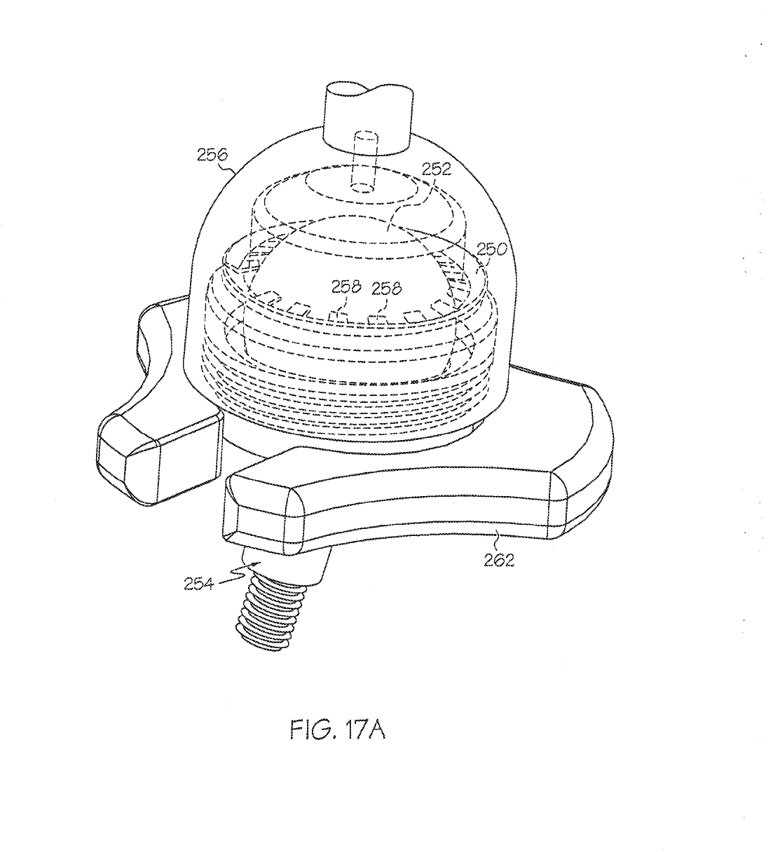

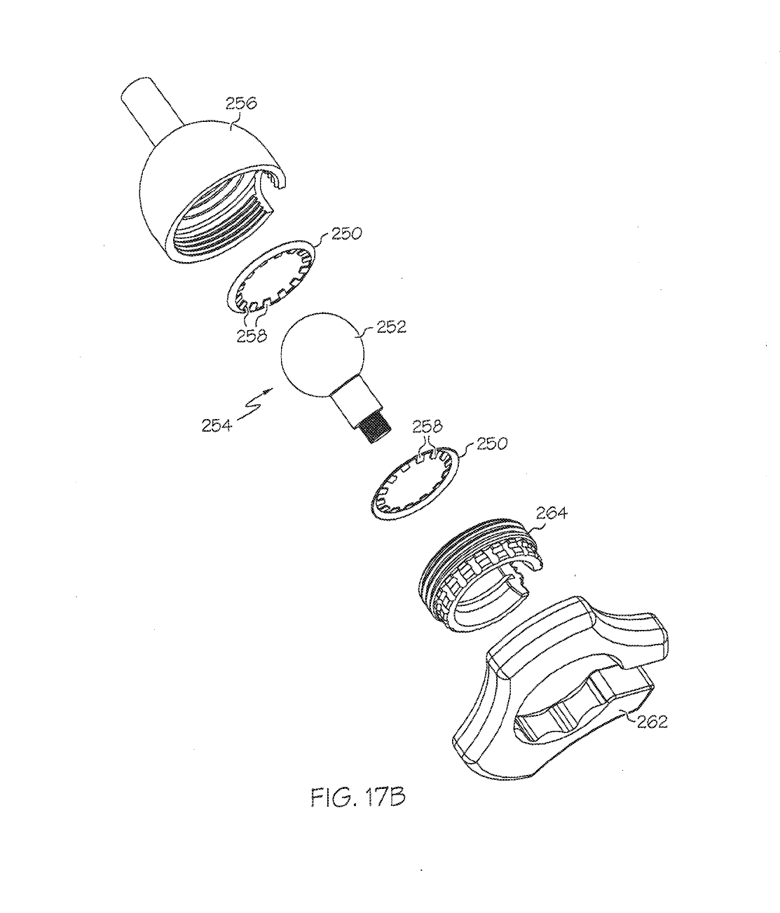

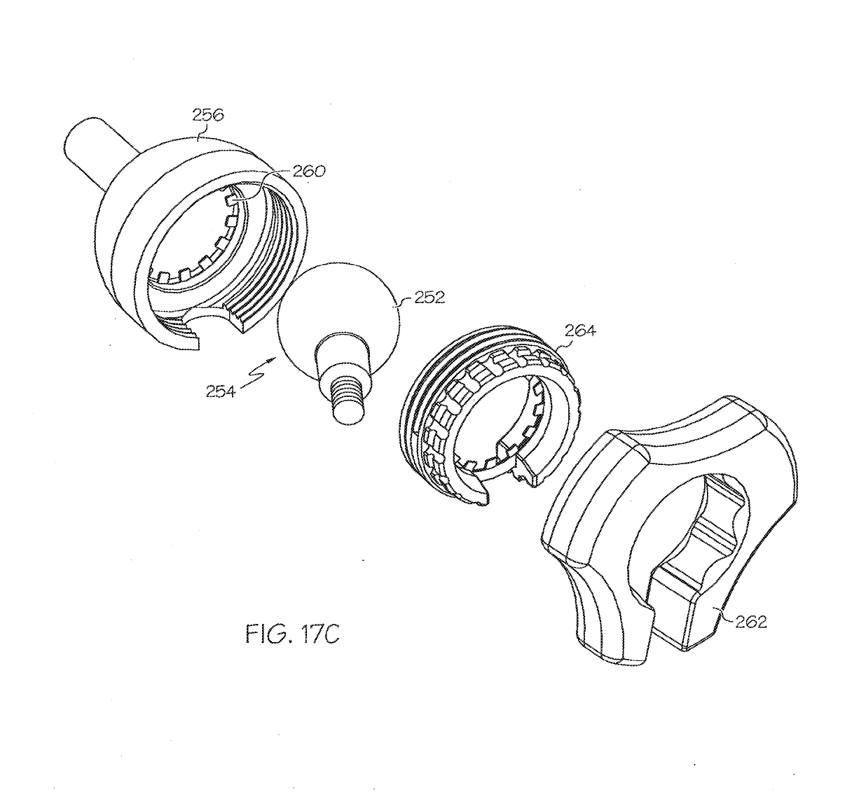

As those of skill in the art will understand and appreciate herein, various different means can be utilized for provisionally and definitively locking any of the poly polyaxial pivot housings (e.g., 120, 160, 161 and 220) disclosed within of the present application. For instance, as shown in FIGS. 17A-17C, one or more self-locking retaining rings 250 can be mated with a top and/or bottom surfaces of the spherical ball portion 252 of the sphere/post assembly 254. In accordance with this illustrative aspect of the present disclosure, the spherical ball portion 252 is able to rotate freely inside the housing 256 until an axial force drives the ball 252 into the socket. As this force is applied to the ball, the teeth 258 of the one or more retaining rings 250 are caused to dig into the ball, thereby preventing it from further rotation. While the use of a retaining ring or rings may be desirable to arrest movement of the ball 252 in accordance with certain aspects of the present disclosure, it should be understood and appreciated herein that in accordance with other embodiments, a series of teeth or ridges 260 can be fabricated into the inner periphery of the housing 256 to arrest movement of the ball in lieu of (or in addition to) a retaining ring (see FIG. 17C). Despite the means chosen to arrest movement of the ball 252 (e.g., retaining ring or fabricated ridges), to allow a threaded member (not shown) to apply additional compression to the ball 252 and thereby achieve a permanent or definitive lock, a removable wrench 262 can be connected to the compression collar 264 and tightened as explained above to compress the ball 252 within the housing 256.

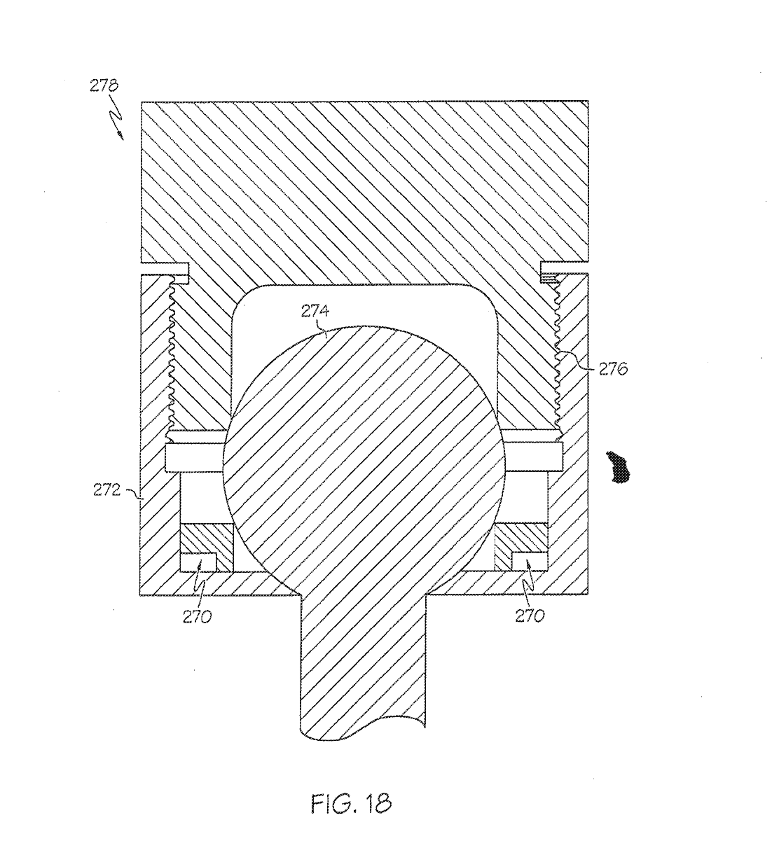

In accordance with yet another embodiment, and as is specifically shown in FIG. 18, a wave spring 270 can be inserted into the housing 272 such that it pinches the ball 274 on each side of its equator to achieve the provisional lock. More particularly, as tension is applied to the frame via a strut, the ball 274 compresses the wave spring 270 so that there is no longer contact with the edge of the socket. Permanent (definitive) fixation can be achieved by tightening the outer threads 276 of the housing 278 until the ball 274 is tightly fixed.

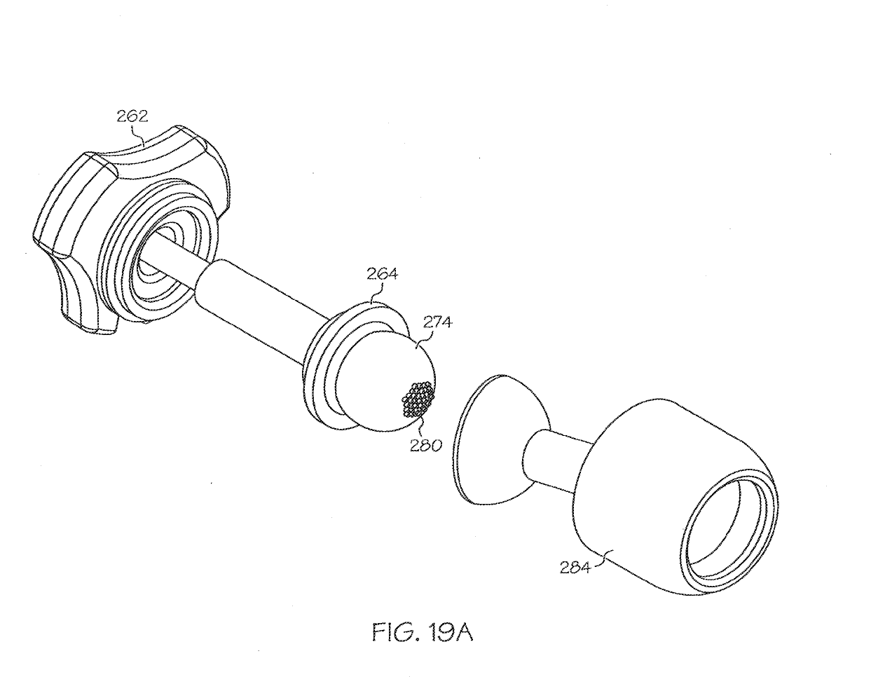

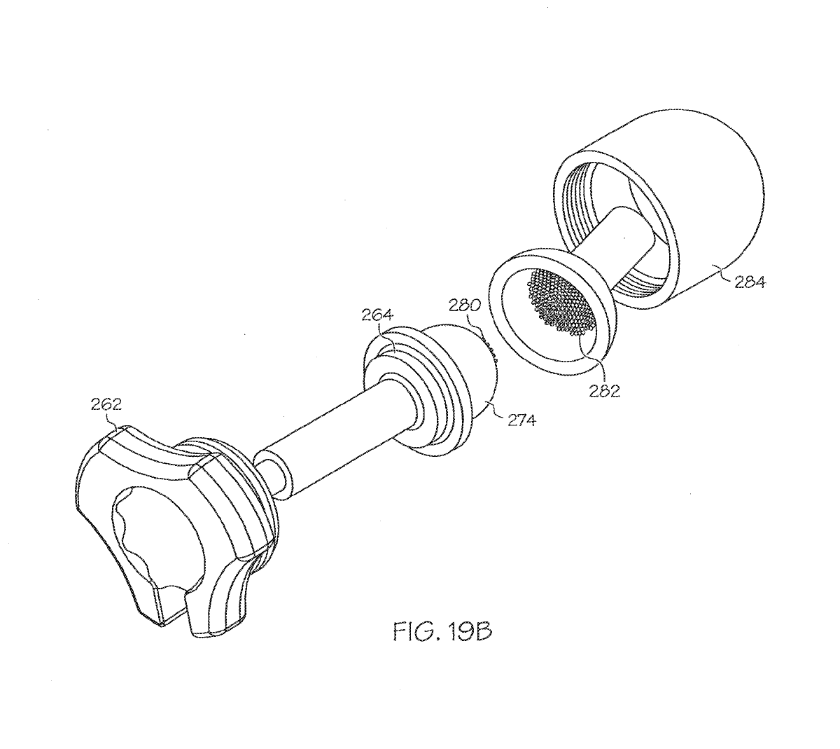

As shown in FIGS. 19A and 19B, instead of pinching the ball 274 with a wave spring, those of skill in the art will understand and appreciate that it is also possible to include protrusions 280 from the ball that are configured to matably engage corresponding recessed portions 282 of the socket 284 to prevent rotation. While not shown specifically herein, it is also possible to prevent rotation by including sharp spikes on the socket that are designed to cut into a semi-soft ball.

In accordance with certain fixation procedures of the present disclosure, it may be desirable to position or stack one or more clamping assemblies on top of each other. While it may be desirable to allow these stacked components to freely articulate with respect to one another in certain situations, in accordance with other aspects of the present teachings, it may be desirable to establish a fixed relationship between these parts. While there are numerous ways to establish a fixed relationship between any of the disclosed clamping assemblies discussed herein, in accordance with certain aspects of the present teachings, the clamping assemblies may have one or more serrated surfaces (see serrated surface 222 of FIG. 3C, for instance) that are configured to structurally mesh with one another when desired such that rotational independence of the stacked assemblies is not possible.

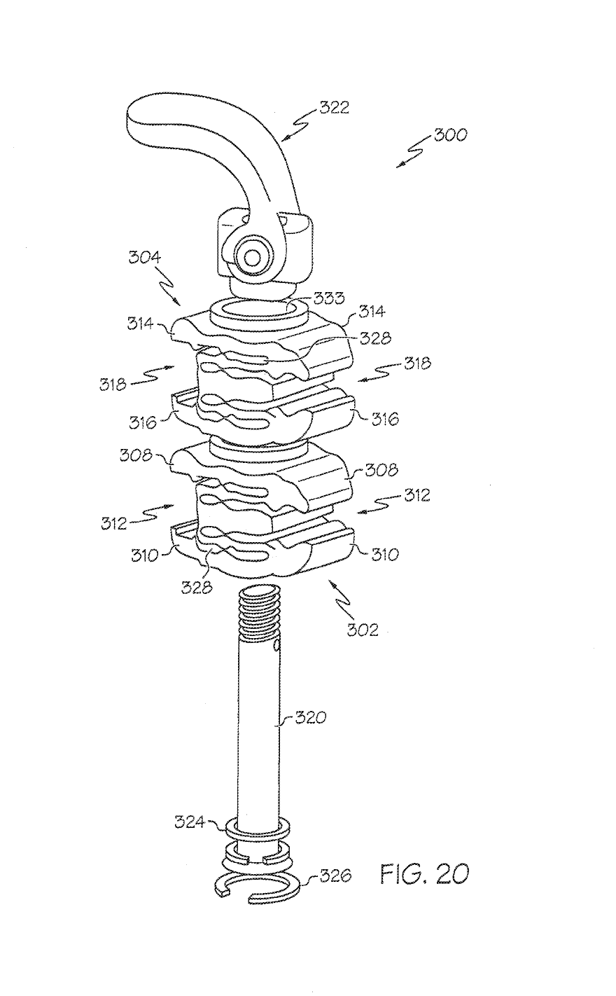

An illustrative embodiment of a stacked or combination rod/pin clamping assembly 300 in accordance with the present disclosure is shown with particular reference to FIG. 20. The clamping assembly 300 in accordance with this aspect of the present disclosure is operative for connecting various elongated members having a cylindrical shape, such as, for instance rods and pins. The clamp assembly 300 is illustrated to include a first or lower clamp member 302 and a second or upper clamp member 304. As used herein, terms of orientation, including but not limited to such as “upper” and “lower” are included merely for purposes of referencing the drawings and are not to be considered limiting in nature. Explaining further, it will be readily apparent to those skilled in the art that any of the disclosed clamping assemblies (including clamp assembly 300, for instance) are contemplated to be equally operative in any conceivable orientations with respect to one another, and as such, the various illustrative orientations shown within the drawings are not intended to serve as an all-inclusive list of the available orientations to which these components can be used in conjunction with the presently disclosed external fixation frame systems.

The first clamp member 302 includes a pair of upper jaw portions 308 and a pair of lower jaw portions 310 which cooperate to define respective openings 312 for receiving cylindrical rods and/or pins that are needed to assemble the external fixation system. Similarly, the second clamp member 304 includes a pair of upper jaw portions 314 and a pair of lower jaw portions 316 which cooperate to define respective openings 318 for receiving cylindrical rods and/or pins inserted therein as well.

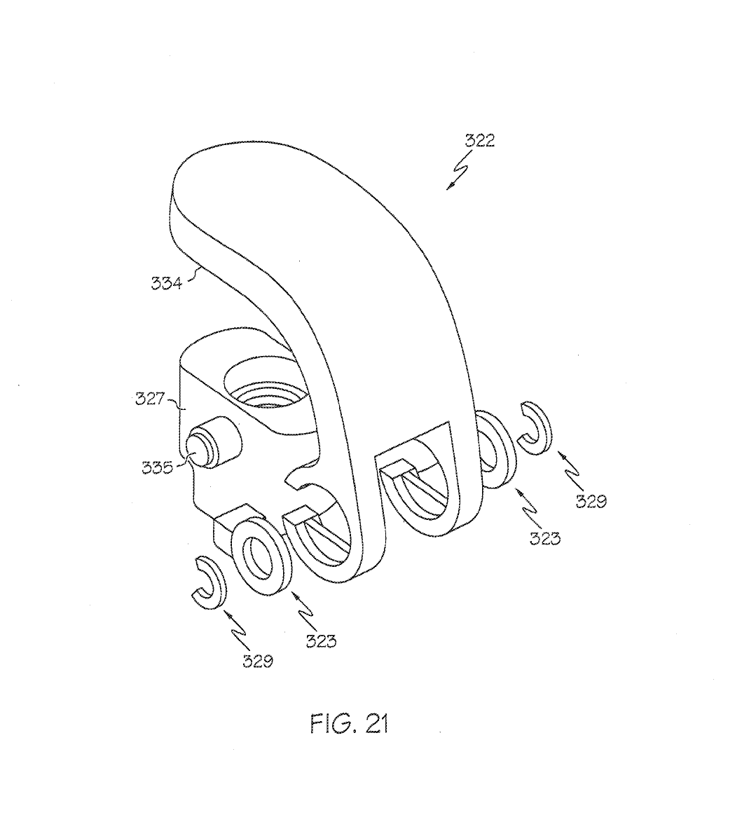

In accordance with this illustrative embodiment, the first and second clamp members 302, 304 are connected to one another by a threaded clamp bolt 320 that passes through a common aperture 333 of the clamp members in such a manner that it compresses the first and second clamp members 302, 304 against one another, as well as the rods and/or pins held therein. The threaded clamp bolt 320 is configured to thread into a cam locking nut assembly 322 that is housed within an opening on the top surface of the second clamp member 304. As shown in FIG. 21, the cam locking nut assembly 322 includes a body 327 that interfaces with the threaded clamp bolt 320 to provide provisional locking, as well as a pivot arm 334 which actuates the definitive lock with movement of a pair of offset cam rings 323. The offset cam rings 323 in turn transmit motion of the pivot arm 334 around pivot posts 335 to a definitive locking force against the first and second clamp members 302, 304 as they are centered by the threaded clamp bolt 320. The offset cam rings 323 and the cam lock pivot arm 334 are held on the pivot posts 335 and into the cam locking nut body 327 by way of a pair of external locking rings 329.

Once the various rods and/or pins are positioned within the first and second clamp members 302, 304, the clamps may be provisionally locked by turning (rotating) the cam locking nut assembly 322 around the threaded clamp bolt rod 320 until the assembly 300 is finger tight. As the threaded clamp bolt 320 is tightened, an O-ring 324 acts as a spring, while an internal snap ring 326 holds the clamp bolt 320 and O-ring 324 inside of the lower section of the first clamp member 302. Once the surgeon is satisfied with the position and fixation of the bone fragments, the assembly 300 can be definitively locked by rotating the pivot arm 334 of the cam locking nut assembly 322 in a direction that moves the offset cam rings 323 into contact with the opposing items, thereby creating an additional offset distance and additional locking load.

In accordance with certain aspects of the present teachings, the first and second clamp members 302, 304 may have a series of axial slots 328 formed into their respective bodies. According to certain aspects of this embodiment, the axial slots may be positioned adjacent to and/or at least partially terminating into the openings 312, 318 to thereby allow the jaw portions of the clamp members to be elastically displaced in response to a cylindrical rod, pin or the like being introduced therein. In other words, as a rod, pin, etc. is laterally introduced into one of the openings 312, 318, the upper and lower jaw portions of that clamping member are urged apart from one another. Once the cylindrical object is fully seated within the upper and lower jaw portions of the clamping member, a counter-force snappingly retains the object temporarily in place, particularly as the upper and lower jaw portions return to their original position prior to being displaced. As those of skill in the art will understand and appreciate herein, by fabricating such axial slots 328 into the clamping assembly, the elastic deformation properties associated with such a design inherently allows the upper and lower jaw portions to function much like a leaf spring, and as a result, cylindrical items placed therein can be snappingly retained within the defined opening.

To provide means for arresting rotational movement of the first clamp member 302 relative to the second clamp member 304 about a longitudinal axis of the clamp bolt 320, the first and second clamp members 302 and 304 may be formed to include cooperating serrations. As shown by reference numeral 222 in FIG. 3C for instance, an upper surface of the first clamp member 302 can similarly include a serrated portion having a plurality of serrations radially extending from a common aperture. The serrated portion can be adapted to engage a substantially identical serrated portion provided on an adjacent lower surface of the second clamp member 304. When the clamp bolt 320 compresses the first and second clamp member 302, 304 against each other as the cam locking nut assembly 322 is initially tightened, the serrated portions of the first and second clamp members 302 and 304 are drawn together to prevent relative movement therebetween.

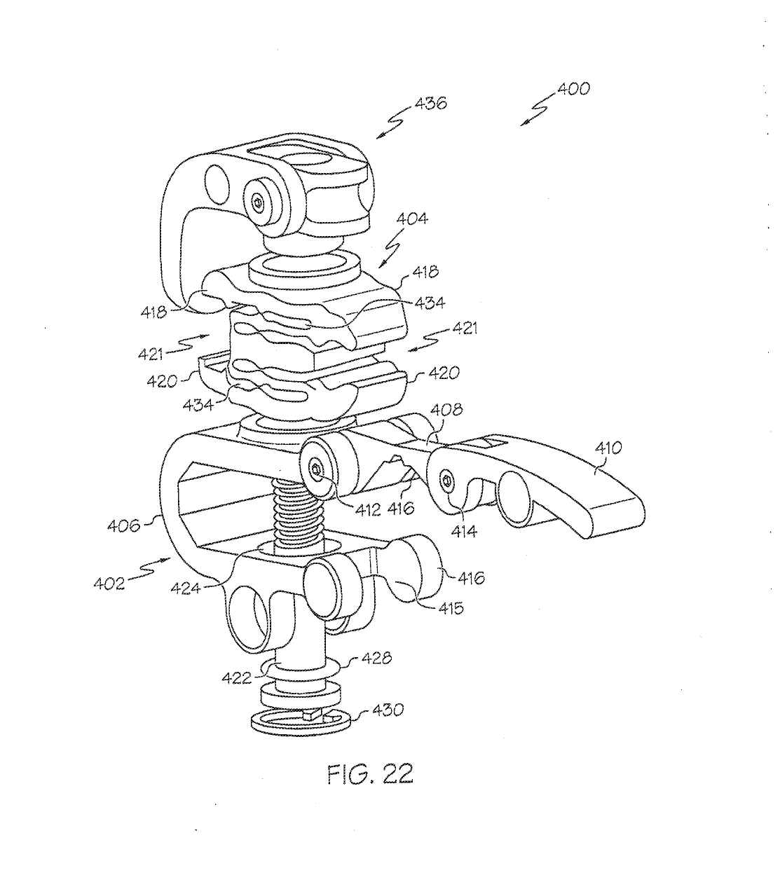

Moving now to FIG. 22, a universal ring frame to rod/pin clamp assembly 400 in accordance with another teaching of the present disclosure is depicted. The clamp assembly 400 is illustrated to include a first or lower clamp member 402 and a second or upper clamp member 404. The first clamp member 402 includes a clamp body 406, a locking arm 408, a cam arm 410, a locking arm pivot pin 412 and a cam arm pivot pin 414. The first clamp member 402 is configured to be snapped onto a ring frame 102,104 or a rod by positioning the clamp body 406 substantially perpendicular to the ring leg or rod and applying pressure to force end jaws 416 open and over the ring or rod. Alternatively, the first clamp member 402 can be snapped onto the ring frame or rod by positioning the clamp body 406 at the end of a ring leg or rod and applying pressure to slide the body onto the ring or rod.

The second clamp member 404 includes a pair of upper jaw portions 418 and a pair of lower jaw portions 420 which cooperate to define openings 421 for receiving cylindrical rods and/or pins that are needed to assemble the external fixation system.

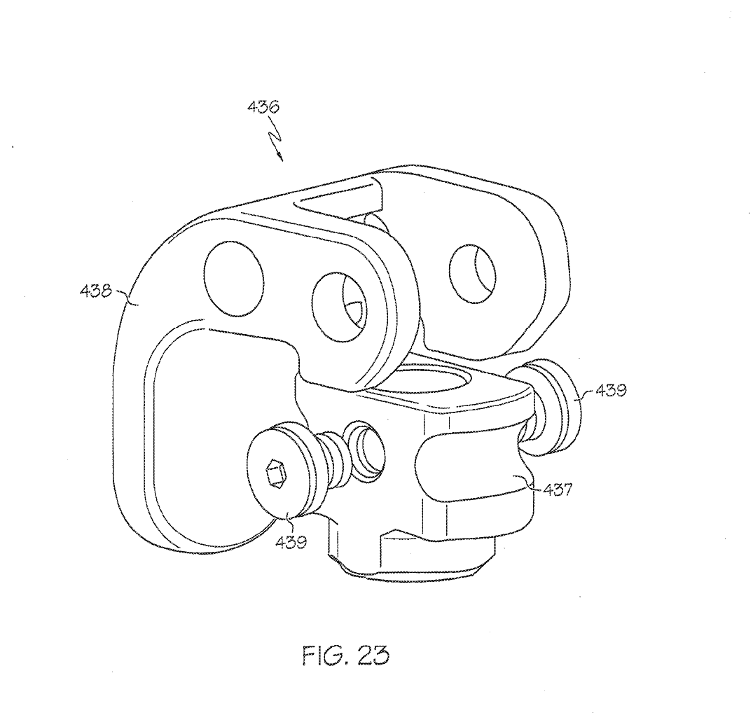

In accordance with this illustrative embodiment, the first and second clamp members 402, 404 are connected to one another by a threaded clamp bolt 422 that passes through a common aperture 424 of the clamp members in such a manner that it compresses the first and second clamp members 402, 404 against one another, as well as the rings, rods and/or pins held therein. The threaded clamp bolt 422 is configured to thread into a cam locking nut assembly 436. As shown in FIG. 23, the cam locking nut assembly 436 includes a body 437 that interfaces with the threaded clamp bolt 422 to provide provisional locking, as well as an off-set cam pivot arm 438 which pivots around a pair of low profile or bottom head cap screws 439.

Once the various rods, pins and/or rings are positioned within the first and second clamp members 402, 404, the clamps may be provisionally locked by turning the cam locking nut assembly 436 around the threaded clamp bolt rod 422 until the assembly 400 is finger tight. As the threaded clamp bolt 422 is tightened, an O-ring 428 acts as a spring, while an internal snap ring 430 holds the clamp bolt 422 and O-ring 428 inside of the lower section of the first clamp member 402. Moreover, the locking arm 408 of the first clamp member 402 is rotated (via the locking arm pivot pin 412) towards and into a cam arm pocket 415 to further create a provisional lock with respect to the first clamp member.

Once the surgeon is satisfied with the position and fixation of the bone fragments, the assembly can be definitively locked by rotating the pivot arm 438 of the cam locking nut assembly 436 to create an additional offset distance and additional locking load, as well as by rotating the cam arm 410 of the first clamp member 402 towards the clamp body 406 via the cam arm pivot pin 414 located at the center of the cam arm 410 until it touches the clamp body 406.

In accordance with certain aspects of the present teachings, the second clamp member 404 may have a series of axial slots 434 formed into its body. According to certain aspects of this embodiment, the axial slots may be positioned adjacent to and/or at least partially terminating into the openings 421 to thereby allow the jaw portions 418, 420 to be elastically displaced in response to a cylindrical rod, pin or the like being introduced therein. In other words, as a rod, pin, etc. is laterally introduced into one of the openings 421, the upper and lower jaw portions defining that opening are urged apart from one another. Once the cylindrical object is fully seated within the upper and lower jaw portions, a counter-force snappingly retains the object temporarily in place, particularly as the upper and lower jaw portions return to their original position prior to being displaced. As those of skill in the art will understand and appreciate herein, by fabricating such axial slots 434 into the clamp member, the elastic deformation properties associated with such a design inherently allows the upper and lower jaw portions to function much like a leaf spring, and as a result, cylindrical items placed therein can be snappingly retained within the defined opening.

To provide means for arresting rotational movement of the first clamp member 402 relative to the second clamp member 404 about a longitudinal axis of the clamp bolt 422, the first and second clamp members 402 and 404 may be formed to include cooperating serrations as described above with respect to assembly 201.

Referring to FIG. 24, a universal ring-to-post clamp assembly 500 is provided. In accordance with this embodiment, the assembly 500 comprises a post body 502 that interfaces with the ring clamp body 504. More particularly, the clamping assembly 500 includes a clamp body 504, a locking arm 506, a cam arm 508, a locking arm pivot pin 510, a cam arm pivot pin 512, and a post body 502 that interfaces with the clamp body 504.

In accordance with this illustrative embodiment, the universal post body 502 and the ring clamp body 504 are connected to one another by a threaded clamp bolt 522 that passes through a common aperture 523 of the bodies 502, 504 in such a manner that it compresses the bodies against one another, as well as the ring held therein. The threaded clamp bolt 522 is configured to thread into a cam locking nut assembly 436 that is housed within an opening on the top surface of the universal post body 502.

Once the ring frame is positioned within the ring clamp body 504, the clamp may be provisionally locked by turning the cam locking nut assembly 436 around the threaded clamp bolt rod 522 until the assembly 500 is finger tight. As the threaded clamp bolt 522 is tightened, an O-ring 528 acts as a spring, while an internal snap ring 530 holds the clamp bolt 522 and O-ring 528 inside of the lower section of the clamp body 504. Moreover, the locking arm 506 of the ring clamp body 502 is rotated (via the locking arm pivot pin 510) towards and into a cam arm pocket 515 to further create a provisional lock with respect to the first clamp member.

Once the surgeon is satisfied with the position and fixation of the bone fragments, the assembly can be definitively locked by rotating the pivot arm 438 of the cam locking nut assembly 436 to create an additional offset distance and locking load, as well as by rotating the cam arm 508 towards the ring clamp body 502 via the cam arm pivot pin 512 located at the center of the cam arm 508 until it touches the clamp body 502.

To provide means for arresting rotational movement of the first and second bodies, 502, 504 relative to one another about a longitudinal axis of the clamp bolt 522, the first and second bodies 502, 504 may be formed to include cooperating serrations as described above with respect to assembly 201.

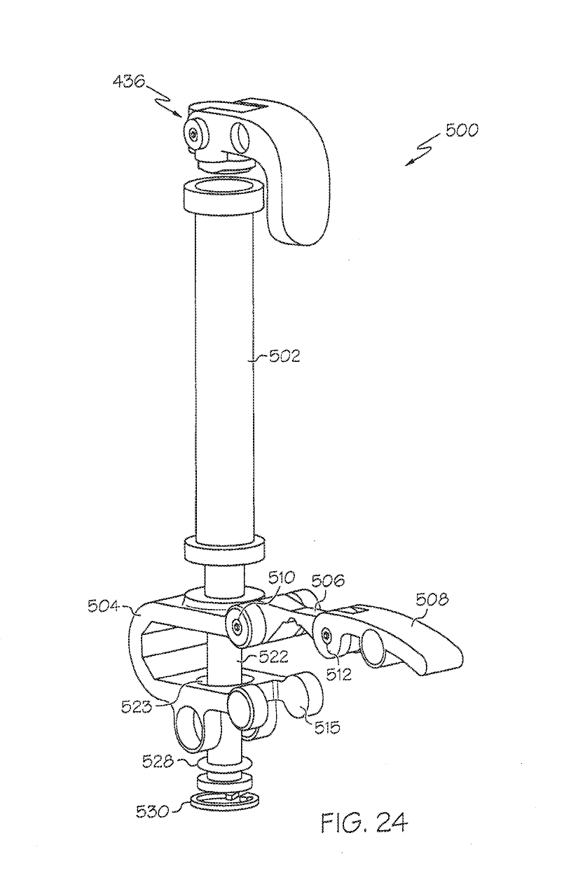

Referring to FIG. 25, another illustrative clamping assembly 600 in accordance with the present disclosure is illustrated. This illustrative assembly is similar to clamping assembly 300 illustrated in FIG. 20 yet includes two cam locking nut assemblies in conjunction with a centralized serrated washer component. In accordance with this aspect of the present disclosure, the clamping assembly 600 is operative for connecting various elongated members having a cylindrical shape, such as, for instance rods and pins. The clamp assembly 600 is illustrated to include a first or lower clamp member 602 and a second or upper clamp member 604. The first clamp member 602 includes a pair of upper jaw portions 608 and a pair of lower jaw portions 610 which cooperate to define respective openings 612 for receiving cylindrical rods and/or pins that are needed to assemble the external fixation system. Similarly, the second clamp member 604 includes a pair of upper jaw portions 614 and a pair of lower jaw portions 616 which cooperate to define respective openings 618 for receiving cylindrical rods and/or pins inserted therein as well.

In accordance with this illustrative embodiment, the first and second clamp members 602, 604 are spaced apart or separated from one another by a serrated washer assembly 621 that holds and contains first and second threaded clamp bolts 622A, 622B that are each configured to independently pass through an aperture formed within one of the respective clamp members 602, 604. In particular, the serrated washer assembly 621 has a first serrated surface 625A and an opposing second serrated surface 625B, the first and second serrated surfaces being substantially parallel to one another. The first and second serrated surfaces 625A, 625B each include a substantially circular recessed portion or cavity (not particularly shown) that is configured to receive an O-ring 633A, 633B associated with the respective first and second threaded clamp bolts 622A, 622B. The O-rings 633A, 633B are in turn compressed against the threaded clamp bolts 622A, 622B by washers 628A, 628B.

The first and second threaded clamp bolts 622A, 622B are each configured to thread into a cam locking nut assembly 436 that is respectively housed within an opening of the first and second clamp members 602, 604. As shown in FIG. 23, the cam locking nut assemblies 436 include a body 437 that interfaces with the threaded clamp bolts 622A, 622B to provide provisional locking, as well as a locking pivot arm 438 which pivots around a low profile or button head cap screws 439 and has an off-set cam that adds distance for tightening the threaded clamp bolts 622A, 622B.

Once the various rods and/or pins are positioned within the first and second clamp members 602, 604, the clamps may be provisionally locked by turning the respective cam locking nut assemblies 436 around the threaded clamp bolts 622A, 622B until the assembly 600 is finger tight. As the threaded clamp bolts 622A, 622B are tightened, the O-rings 633A, 633B act as springs, while internal snap rings 640a, 640b hold the clamp bolts 622A, 622B, O-rings 633A, 633B and washers 628a, 628b inside of the serrated washer assembly 621. Once the surgeon is satisfied with the position and fixation of the bone fragments, the assembly 600 can be definitively locked by rotating the locking pivot arms 638 of the cam locking nut assemblies 436 in a direction that moves the offset cam rings into contact with the opposing item, thereby creating an additional offset distance and additional locking load.

In accordance with certain aspects of the present teachings, the first and second clamp members 602, 604 may have a series of axial slots 642 formed into their respective bodies. According to certain aspects of this embodiment, the axial slots may be positioned adjacent to and/or at least partially terminating into the openings 612, 618 to thereby allow the jaw portions of the clamp members to be elastically displaced in response to a cylindrical rod, pin or the like being introduced therein. In other words, as a rod, pin, etc. is laterally introduced into one of the openings 612, 618, the upper and lower jaw portions of that clamping member are urged apart from one another. Once the cylindrical object is fully seated within the upper and lower jaw portions of the clamping member, a counter-force snappingly retains the object temporarily in place, particularly as the upper and lower jaw portions return to their original position prior to being displaced. As those of skill in the art will understand and appreciate herein, by fabricating such axial slots 642 into the clamping assembly, the elastic deformation properties associated with such a design inherently allows the upper and lower jaw portions to function much like a leaf spring, and as a result, cylindrical items placed therein can be snappingly retained within the defined opening.

To provide means for arresting rotational movement of the first clamp member 602 relative to the second clamp member 604 about a longitudinal axis of the clamp bolts 622A, 622B, the first and second clamp members 602 and 604 may be formed to include cooperating serrated portions or surfaces that are configured to interact with the serrated surfaces 625A, 625B of the serrated washer assembly 621. The serrated portions of the first and second clamp members 602, 604 can be adapted to engage the serrated surfaces 625A, 625B of the serrated washer assembly 621 such that when the clamp bolts 622A, 622B compress the first and second clamp member 602, 604 against the serrated washer assembly 621 as the cam locking nut assemblies 636A, 636B are initially tightened, the serrated portions of the first and second clamp members 602 and 604 are drawn against the serrated surfaces 625A, 625B of the washer assembly such that relative movement between the first and second clamp members is prevented.

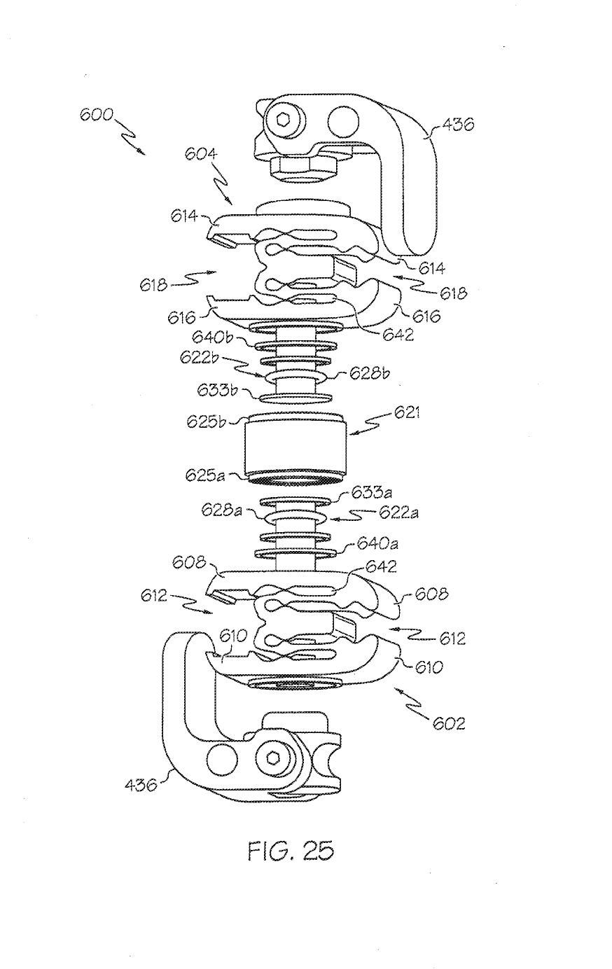

Referring to FIG. 26, an illustrative universal rod/pin clamping assembly 700 in accordance with the present disclosure is illustrated. The clamping assembly 700 is illustrated to include a first or lower clamp member 702 and a second or upper clamp member 704. The first clamp member 702 includes a pair of upper jaw portions 708 and a pair of lower jaw portions 710 which cooperate to define respective openings 712 for receiving cylindrical rods and/or pins that are needed to assemble the external fixation system. Similarly, the second clamp member 704 includes a pair of upper jaw portions 714 and a pair of lower jaw portions 716 which cooperate to define respective openings 718 for receiving cylindrical rods and/or pins inserted therein as well.

In accordance with this illustrative embodiment, the first and second clamp members 702, 704 are coupled to one another by a pair of locking assemblies 721A, 721B that are each independently configured to pivot with respect to one another, as well as with respect to the clamp members 702, 704. In accordance with this aspect of the present disclosure, the bottom surface of a second pivot body 719B of the second locking assembly 721B is configured to rest upon the top surface of a first pivot body 719A of the first locking assembly 721A. Both the first and second pivot bodies 719A, 719B each have a through-hole (not shown) such that when they are stacked on top of each other, a common through-hole is created. Within this common through-hole is housed a universal serrated washer 725. The universal serrated washer 725 has a pair of opposing serrated surfaces 725A (only one surface shown), as well as a pair of retaining ring grooves (not shown). When assembled, a pair of retaining rings 723A, 723B is inserted within the grooves and serve as a means for preventing the first and second pivot bodies 719A, 719B from disengaging from one another.

The first and second clamp members 702, 704 are coupled to first and second threaded clamp bolts 724A, 724B that are each configured to independently pass through apertures which are formed in the respective clamp members 702, 704. The first and second threaded clamp bolts 724A, 724B are each configured to thread into a threaded aperture (not shown) of the serrated washer 725.

Once the various rods and/or pins are positioned within the first and second clamp members 702, 704, the clamps may be provisionally and independently locked by rotating their respective locking pivot arm 738A, 738B (via the locking arm pivot pins 740A, 740B) towards and into cam arm pockets. Once the surgeon is satisfied with the position and fixation of the bone fragments, the frame or fixator can be definitively locked without the use of additional tools or equipment. To achieve the definitive lock, the cam arms 739A, 739B are rotated towards the clamp members 702, 704 via the cam arm pivot pins 741A, 741B located at the center of the respective cam arms 739A, 739B until it touches the clamp member.

Once the cam arms 739A, 739B are positioned within the clamp members 702, 704 during a definitive locking process, in accordance with certain aspects of the present disclosure, the cam arms 739A, 739B can be further locked into place by utilizing a locking pin (not shown) that is configured to be inserted through apertures 743A, 743B of the cam arms 739A, 739B. According to this embodiment, the clamp members 702, 704 each have a pair of upwardly projecting tabs 742A, 742B, each having a through-hole formed therein. When the cam arms 739A, 739B are positioned within the clamp members 702, 704, the through-holes align with apertures 743A, 743B formed into the cam arms 739A, 739B such that a common through-hole is created. The locking pin can then be inserted through this common through-hole, thereby preventing the cam arms 739A, 739B from being individually lifted away from the clamp members 702, 704 until the locking pin is first removed.

To provide means for arresting rotational movement of the first clamp member 702 relative to the second clamp member 704 about a longitudinal axis of the clamp bolts 724A, 724B, the first and second clamp members 702 and 704 may be formed to include cooperating serrated portions or surfaces that are configured to interact with serrated surface 725A of the serrated washer assembly 725. The serrated portions of the first and second clamp members 702, 704 can be adapted to engage the serrated surfaces of the serrated washer 725 such that when the clamp bolts 724A, 724B compress the first and second clamp member 702, 704, the serrated portions of the first and second clamp members 702 and 704 are drawn against the serrated surface of the serrated washer 725 such that relative movement between the first and second clamp members is prevented.

In accordance with certain aspects of the present teachings, the first and second clamp members 702, 704 may have a series of axial slots 746 formed into their respective bodies. According to certain aspects of this embodiment, the axial slots may be positioned adjacent to and/or at least partially terminating into the openings 712, 718 to thereby allow the jaw portions of the clamp members to be elastically displaced in response to a cylindrical rod, pin or the like being introduced therein. In other words, as a rod, pin, etc. is laterally introduced into one of the openings 712, 718, the upper and lower jaw portions of that clamping member are urged apart from one another. Once the cylindrical object is fully seated within the upper and lower jaw portions of the clamping member, a counter-force snappingly retains the object temporarily in place, particularly as the upper and lower jaw portions return to their original position prior to being displaced. As those of skill in the art will understand and appreciate herein, by fabricating such axial slots 746 into the clamping assembly, the elastic deformation properties associated with such a design inherently allows the upper and lower jaw portions to function much like a leaf spring, and as a result, cylindrical items placed therein can be snappingly retained within the defined opening.

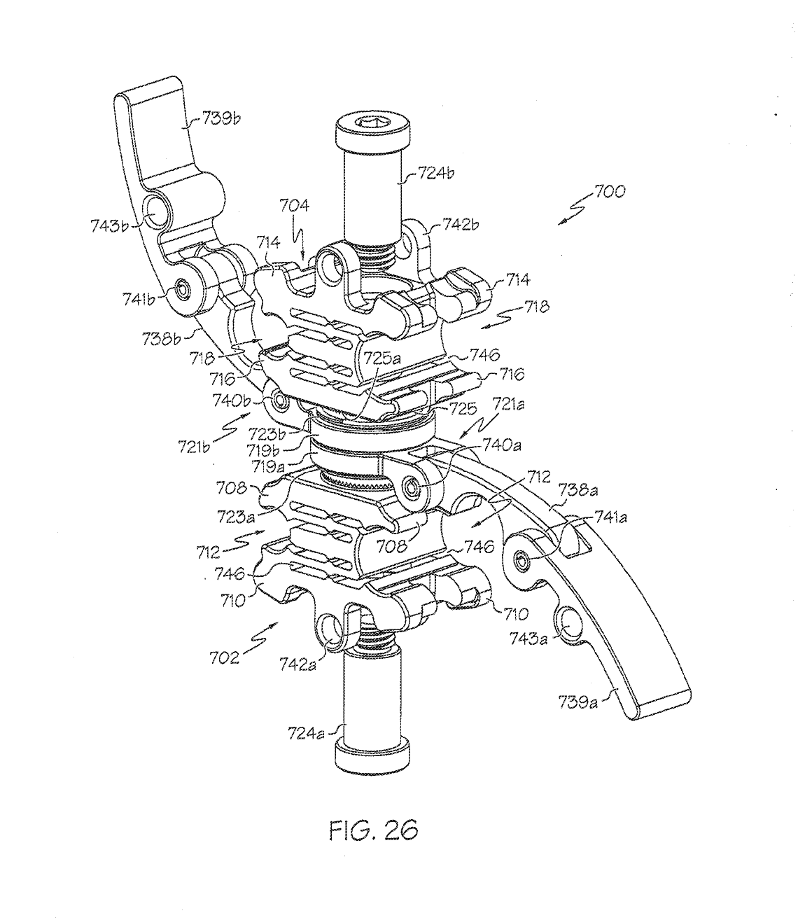

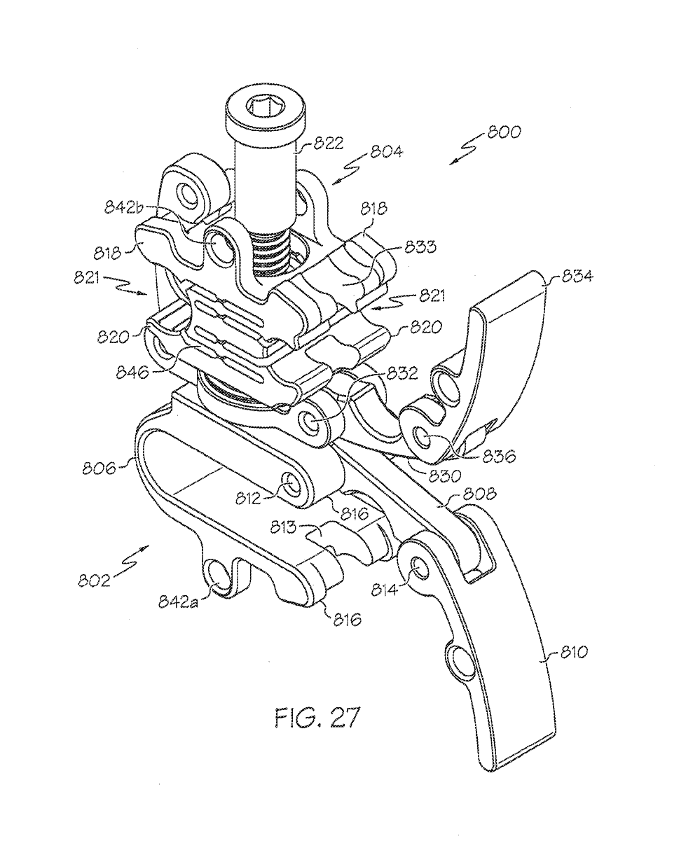

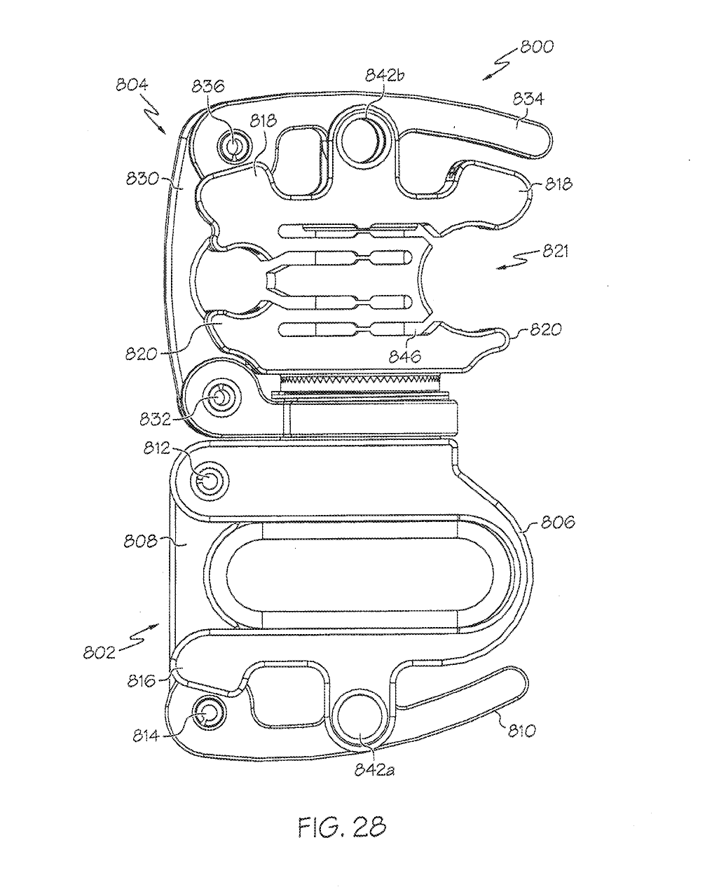

Referring to FIGS. 27 and 28, another illustrative clamping assembly 800 in accordance with the present disclosure is illustrated. In accordance with this aspect of the present disclosure, the illustrative clamping assembly 800 is a universal ring frame to rod/pin clamp assembly. According to this aspect of the present disclosure, the clamp assembly 800 is illustrated to include a first or lower clamp member 802 and a second or upper clamp member 804. The first clamp member 802 includes a clamp body 806, a locking arm 808, a cam arm 810, a locking arm pivot pin 812 and a cam arm pivot pin 814. The first clamp member 802 is configured to be snapped onto a ring frame or a rod (e.g., ring frames 102, 104) by positioning the clamp body 806 substantially perpendicular to the ring leg or rod and applying pressure to force end jaws 816 open and over the ring or rod. Alternatively, the first clamp member 802 can be snapped onto the ring frame or rod by positioning the clamp body 806 at the end of a ring leg or rod and applying pressure to slide the body onto the ring or rod.

The second clamp member 804 includes a pair of upper jaw portions 818 and a pair of lower jaw portions 820 which cooperate to define openings 821 for receiving cylindrical rods and/or pins that are needed to assemble the external fixation system.

In accordance with this illustrative embodiment, the first and second clamp members 802, 804 are connected to one another by a threaded clamp bolt 822 that passes through a common aperture of the clamp members in such a manner that it compresses the first and second clamp members 802, 804 against one another, as well as the rings, rods and/or pins held therein.

Once the various rings, rods and/or pins are positioned within the first and second clamp members 802, 804, first and second locking assemblies compress and hold the rings, rods and/or pins therein. To achieve this, the clamp members may be provisionally and independently locked. To provisionally lock the first clamp member 802, the locking arm 808 is rotated (via the locking arm pivot pin 812) towards and into a cam arm pocket 813. Once the surgeon is satisfied with the position and fixation of the bone fragments, the frame or fixator can be definitively locked without the use of additional tools or equipment. To achieve the definitive lock, the cam arm 810 is rotated towards the clamp body 806 via the cam arm pivot pin 814 located at the center of the cam arm 810 until it touches the clamp body.

Similarly, to independently and provisionally lock the second clamp member 804, a locking arm 830 that is coupled to the second clamp member is rotated (via the locking arm pivot pin 832) towards and into a cam arm pocket 833. Once the surgeon is satisfied with the position and fixation of the bone fragments, the frame or fixator can be definitively locked without the use of additional tools or equipment. To achieve the definitive lock, the cam arm 834 is rotated towards the second clamp member 804 via a cam arm pivot pin 836 located at the center of the cam arm 834 until it touches the second clamp member.

Once the cam arms 810, 834 are positioned within their respective clamp members 802, 804 during a definitive locking process, in accordance with certain aspects of the present disclosure, the cam arms 810, 834 can be further locked into place by utilizing a locking pin (not shown) that is configured to be inserted through the cam arms 810, 834. According to this embodiment, the clamp members 802, 804 each have a pair of upwardly projecting tabs 842A, 842B with through-holes formed therein. When the cam arms 810, 834 are positioned within the clamp members 802, 804, the through-holes align with a through-hole formed into the cam arms 810, 834 such that a common through-hole is created. The locking pin can then be inserted through this common through-hole, thereby preventing the cam arms 810, 834 from being individually lifted away from the clamp members 802, 804 until the locking pin is first removed.

While clamping assembly 800 can be configured such that the first and second clamp members 802, 804 are able to independently rotate with respect to one another, in accordance with certain aspects of the present disclosure, it may be desirable to arrest angular or rotational movement between such components. To provide means for arresting such movement of the first clamp member 802 relative to the second clamp member 804 about an a longitudinal axis of the clamp bolt 822, the first and second clamp members 802 and 804 may be formed to include cooperating serrated portions or surfaces that are configured to interact with each other such that relative movement between the first and second clamp members is prevented.

In accordance with certain aspects of the present teachings, the second clamp member 804 may have a series of axial slots 846 formed into its body. According to certain aspects of this embodiment, the axial slots may be positioned adjacent to and/or at least partially terminating into the openings 821 to thereby allow the jaw portions of the clamp member to be elastically displaced in response to a cylindrical rod, pin or the like being introduced therein. In other words, as a rod, pin, etc. is laterally introduced into one of the openings 821, the upper and lower jaw portions of that clamping member are urged apart from one another. Once the cylindrical object is fully seated within the upper and lower jaw portions of the clamping member, a counter-force snappingly retains the object temporarily in place, particularly as the upper and lower jaw portions return to their original position prior to being displaced. As those of skill in the art will understand and appreciate herein, by fabricating such axial slots 846 into the clamping assembly, the elastic deformation properties associated with such a design inherently allows the upper and lower jaw portions to function much like a leaf spring, and as a result, cylindrical items placed therein can be snappingly retained within the defined opening.

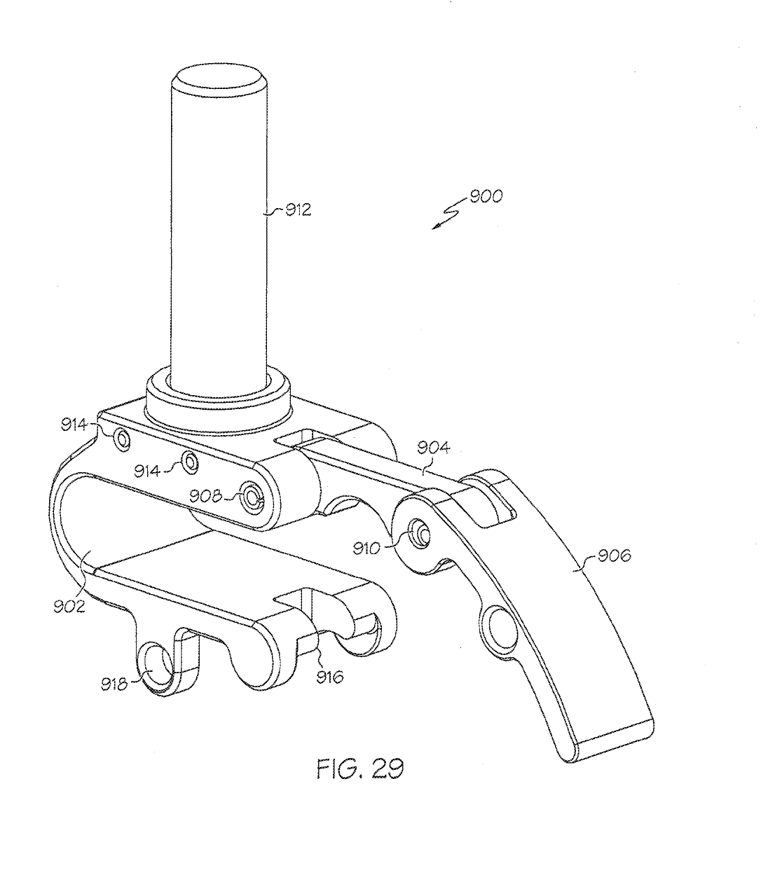

FIG. 29 depicts an illustrative universal ring-to-post clamping assembly 900 in accordance with one embodiment of the present teachings. In accordance with this aspect of the present disclosure, the clamping assembly 900 includes a clamp body 902, a locking arm 904, a cam arm 906, a locking arm pivot pin 908, a cam arm pivot pin 910, a universal post 912 (which is configured to interface with a universal rod/pin clamp assembly) and a retaining pin 914 that retains the universal post into the clamp body 902.

Once the clamp body 902 is positioned on a ring frame, the locking arm 904 compresses and centers the ring frame inside the clamp body 902. To achieve this, the clamp may be provisionally locked by rotating the locking arm 904 (via the locking arm pivot pin 908) towards and into a cam arm pocket 916. Once the surgeon is satisfied with the position and fixation of the bone fragments, the frame or fixator is definitively locked without the use of additional tools or equipment. To achieve the definitive lock, the cam arm 906 is rotated towards the clamp body 902 via the cam arm pivot pin 910 located at the center of the cam arm 906 until it touches the clamp body 902.

Once the cam arm 906 is positioned against the clamp body 902 during a definitive locking process, in accordance with certain aspects of the present disclosure, the cam arm 906 can be further locked into place by utilizing a locking pin (not shown) that is configured to be inserted through the cam arm 906. According to this embodiment, the clamp body 902 has a pair of upwardly projecting tabs 918, each having a through-hole formed therein. When the cam arm 906 is positioned against the clamp body 902, the through-holes align with a through-hole formed into the cam arm 906 such that a common through-hole is created. The locking pin can then be inserted through this common through-hole, thereby preventing the cam arm 906 from being lifted away from the clamp body 902 until the locking pin is first removed.

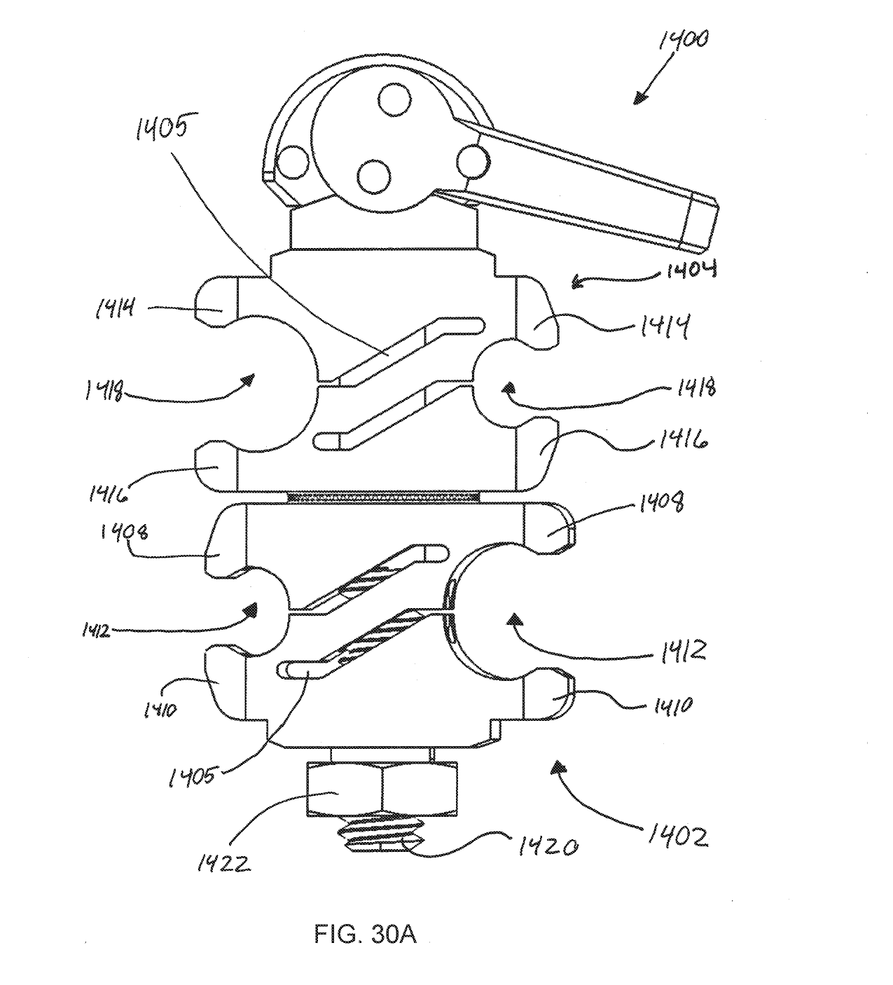

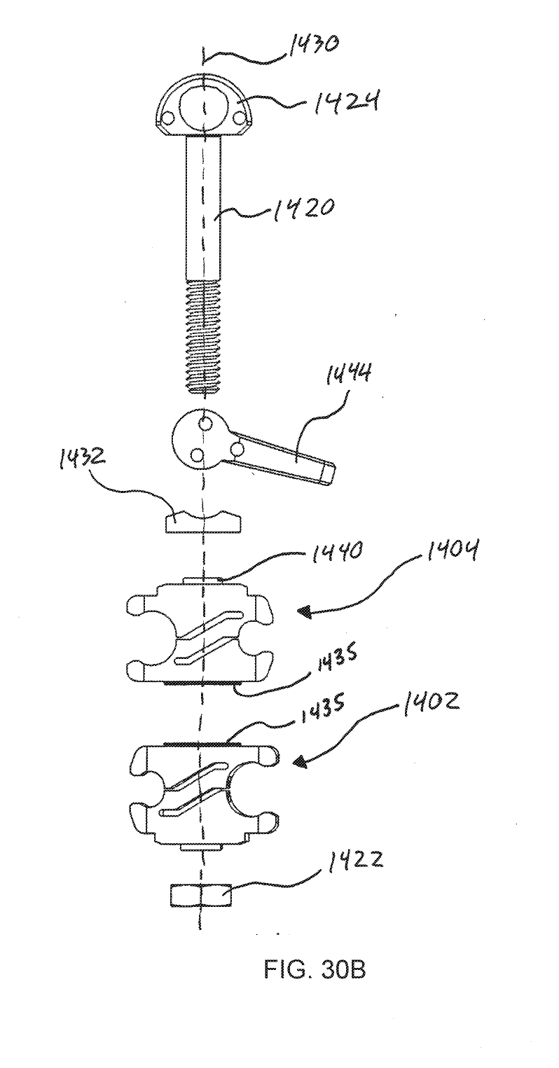

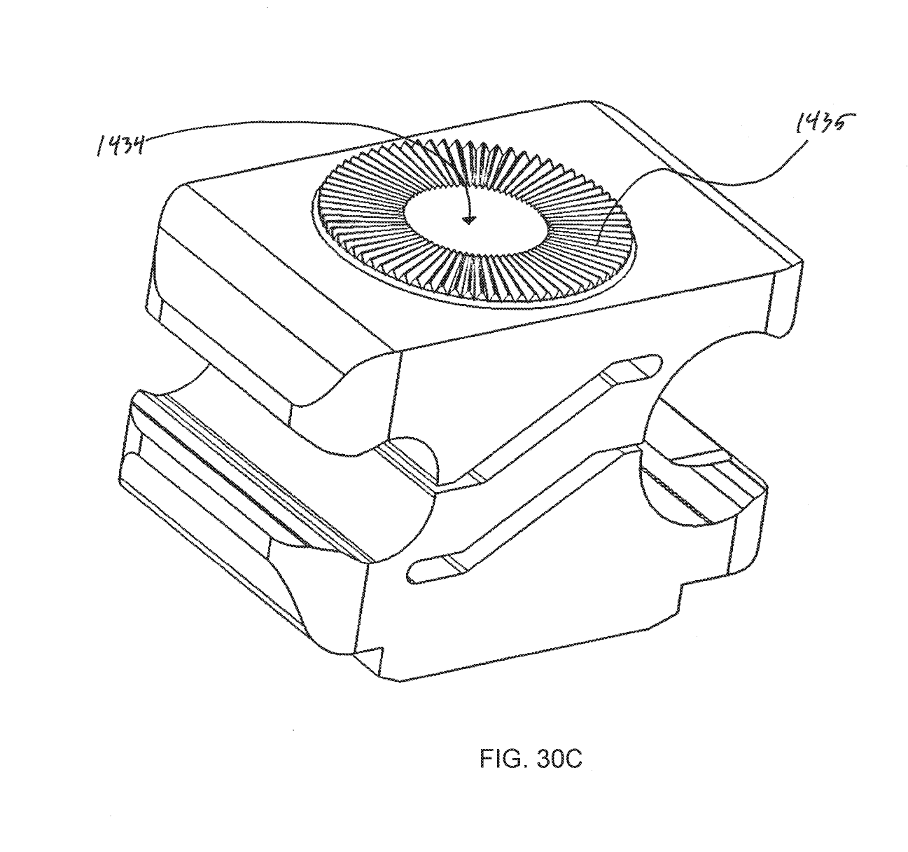

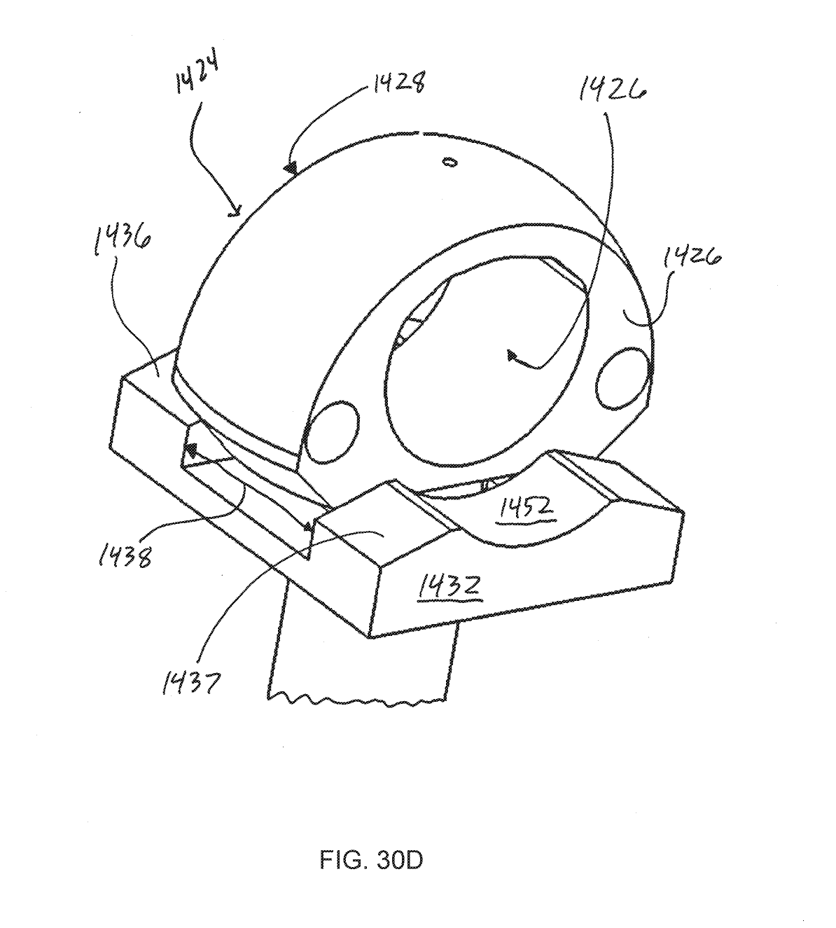

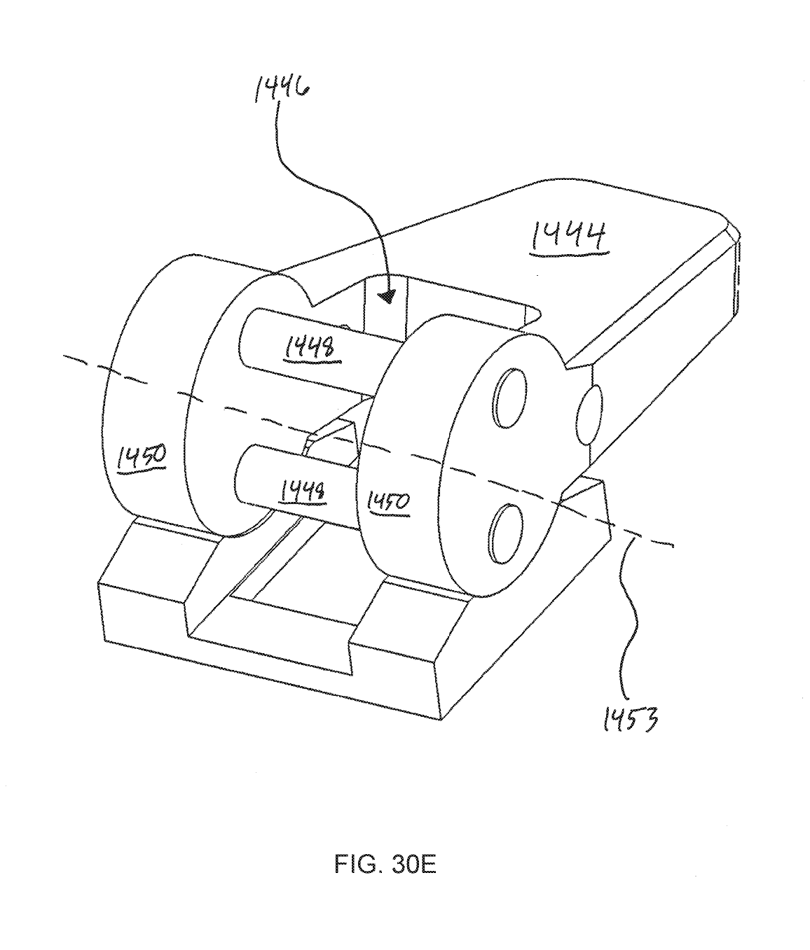

Another embodiment of a stacked or combination clamping assembly 1400 in accordance with the present disclosure is shown with particular reference to FIGS. 30A-30E. The clamping assembly 1400 in accordance with this aspect of the present disclosure is operative for connecting various elongated members having a cylindrical shape, such as, for instance rods and pins. The clamp assembly 1400 is illustrated to include a first or lower clamp member 1402 and a second or upper clamp member 1404.

The first clamp member 1402 includes a pair of upper jaw portions 1408 and a pair of lower jaw portions 1410 which cooperate to define respective openings 1412 for receiving cylindrical rods and/or pins that are needed to assemble the external fixation system. Similarly, the second clamp member 1404 includes a pair of upper jaw portions 1414 and a pair of lower jaw portions 1416 which cooperate to define respective openings 1418 for receiving cylindrical rods and/or pins inserted therein as well.

In accordance with this illustrative embodiment, the first and second clamp members 1402, 1404 are connected to one another by a threaded clamp bolt 1420 that passes through a common aperture 1434 (FIG. 30C) of the clamp members in such a manner that it may compress the first and second clamp members 1402, 1404 against one another, as well as the rods and/or pins held therein. The threaded clamp bolt 1420 is configured to thread into a locking nut 1422 that is located along the bottom surface of the first clamp member 1402.

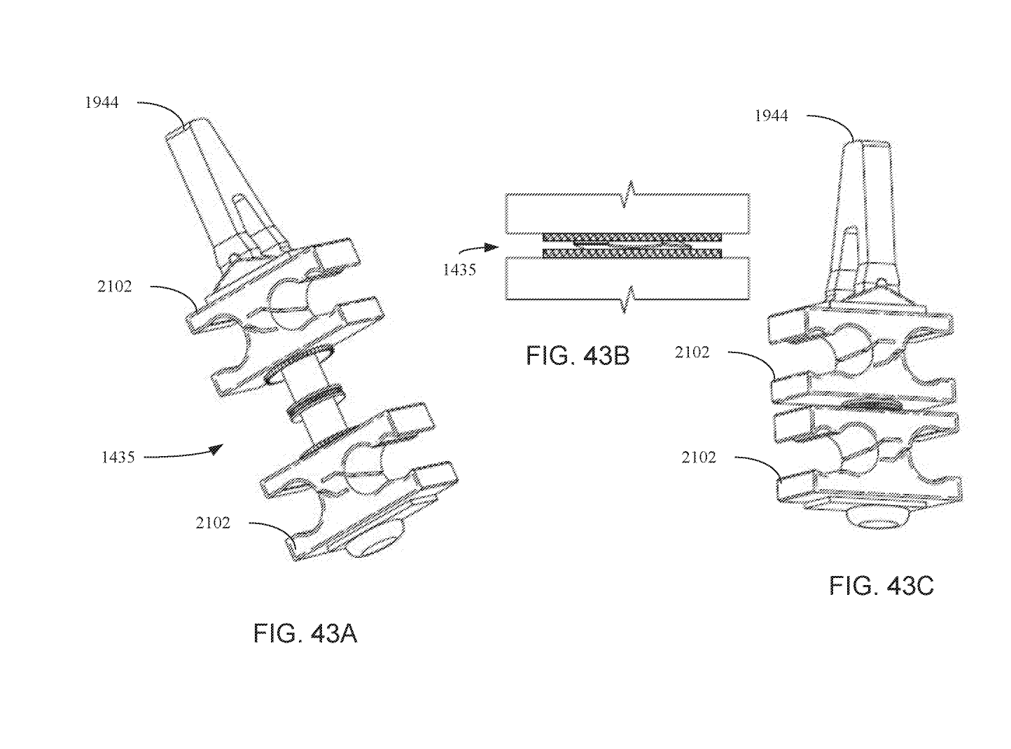

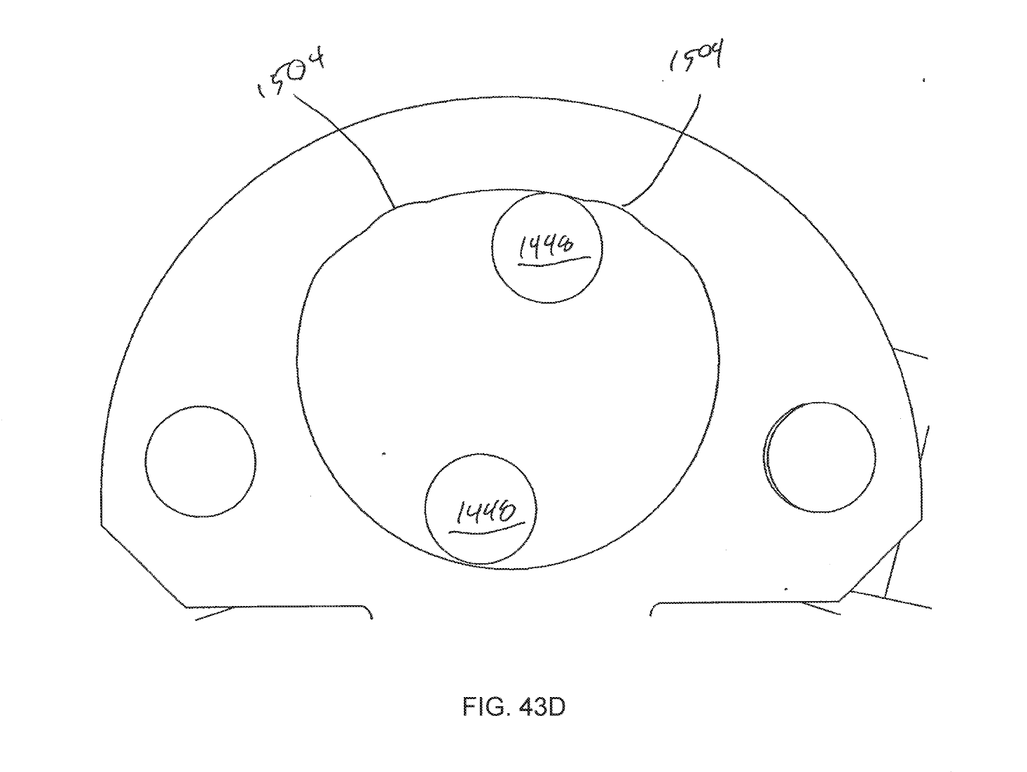

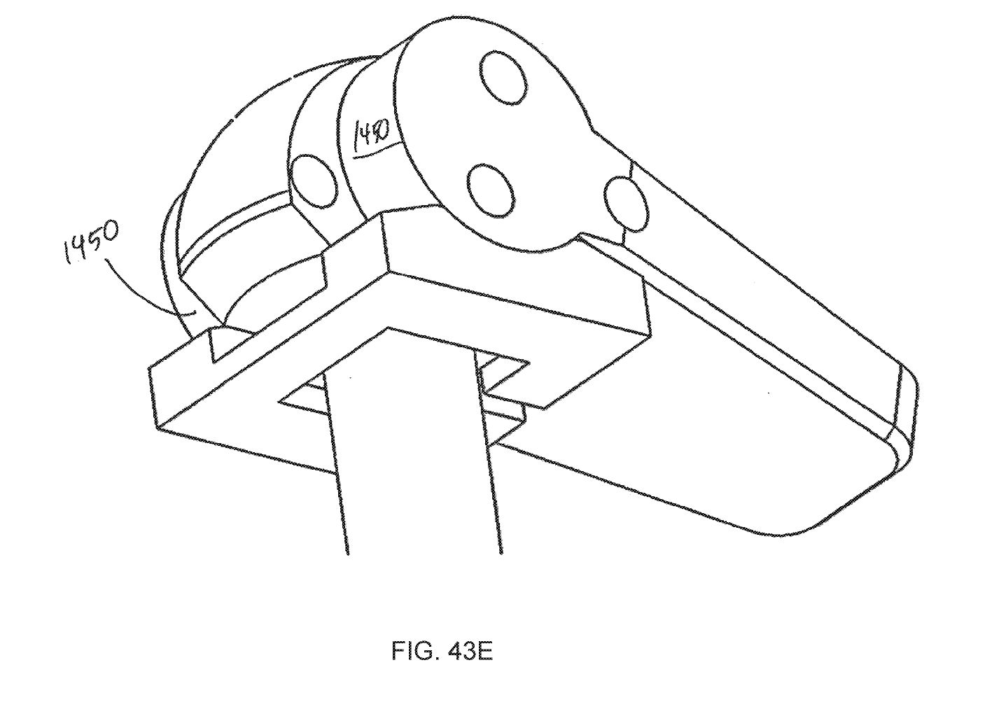

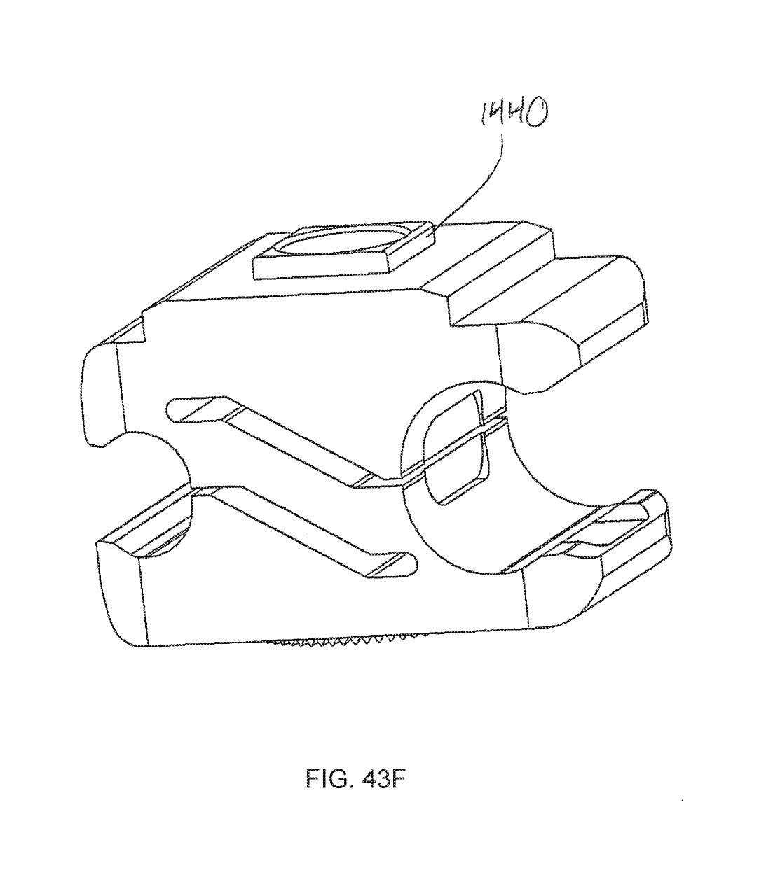

In one aspect of the present disclosure, first and second clamp members 1402, 1404 may have a corresponding serrated portion 1435. The serrated portions 1435 may allow the first and second clamp members 1402, 1404 to be coupled to one another at a plurality of radial orientations relative to one another. In one embodiment, when the first and second clamp 1402, 1404 are not in the compressed orientation, they may be rotated relative to one another to provide a plurality of radial dispositions. In the embodiment shown in FIG. 43, a springing mechanism may be disposed about a radially inner cavity of the serrated portion 1425. The springing mechanism may provide an axial force separating the serrated portions 1425 from one another when the first and second clamp are not in a compressed orientation. Alternatively, when they are transitioned to a compressed orientation, the serrated sections 1435, and the springing mechanism therein, may become compressed toward one another with sufficient force to substantially restrict radial movement, thus locking the radial disposition of the first clamp member 1402 relative to the second clamp member 1404.

In accordance with certain aspects of the present teachings, the first and second clamp members 1402, 1404 may have a series of axial slots 1405 formed into their respective bodies. According to certain aspects of this embodiment, the axial slots 1405 may be positioned adjacent to and/or at least partially terminating into the openings 1412, 1418 to thereby allow the jaw portions of the clamp members to be elastically displaced in response to a cylindrical rod, pin or the like being introduced therein. In other words, as a rod, pin, etc. is laterally introduced into one of the openings 1412, 1418, the upper and lower jaw portions of that clamping member are urged apart from one another. Once the cylindrical object is fully seated within the upper and lower jaw portions of the clamping member, a counter-force snappingly retains the object temporarily in place, particularly as the upper and lower jaw portions return to their original position prior to being displaced. As those of skill in the art will understand and appreciate herein, by fabricating such axial slots 1405 into the clamping assembly, the elastic deformation properties associated with such a design inherently allows the upper and lower jaw portions to function much like a leaf spring, and as a result, cylindrical items placed therein can be snappingly retained within the defined opening.

A clamp bolt head 1424 (FIG. 30B) may be located along the top surface of the second clamp member 1404. Between the bolt head 1424 and the top surface may also be an alignment plate 1432. The alignment plate 1432 may have a first side 1436 (FIG. 30D) and a second side 1437 that are separated by a channel 1438. Further, the bolt head 1424 may have a first face 1426 and a second face 1428 that are parallel to, but offset from, one another. The first and second face 1426, 1428 may also define planes that run parallel to a longitudinal center axis 1430 defined by the threaded clamp bolt 1420. The first and second face 1426, 1428 may be spaced from one another so that they may be at least partially disposed within the channel 1438.

Along the top surface of the second clamp member 1404 may be a protrusion 1440. The protrusion 1440 may have a substantially square or rectangular cross section and protrude only far enough away from the top surface to provide for an alignment mechanism for the alignment plate 1432. The alignment plate 1432 may define a through hole that corresponds with the protrusion 1440 so that the alignment plate 1432 may partially encompass the protrusion 1440 when aligned therewith and placed on the top surface. In this configuration, the alignment plate 1432 may be substantially radially coupled to the protrusion 1440.

When the bolt head 1424 is disposed within the channel 1438 of the alignment plate 1432, which is aligned along the top surface of the second clamp member 1404, the bolt head 1424 may resist radial movement. In one embodiment as a torsional force is applied to the locking nut 1422, the bolt head 1424 may be held in radial alignment with the clamp members because of its disposition within the channel 1438 and further because the alignment plate 1432 may partially encompass the protrusion 1440.

A person having skill in the relevant field of this disclosure understands that radially coupling multiple components can be achieved utilizing a plurality of configurations. While this disclosure specifically refers to a square or rectangular protrusion, the protrusion and corresponding alignment plate could be many other shapes and sizes. In one nonlimiting example the protrusion could be star-shaped, semicircular, a substantially linear wall, or the like. Accordingly, this disclosure should not be limited to any particular size or shape of the protrusion.

Yet another feature of the bolt head 1424 may be a cammed aperture 1442 that corresponds with a locking arm 1444. The cammed aperture 1442 may be defined by the bolt head 1424 and define a contact surface for the locking arm 1444. The locking arm 1444 may have a substantially U-shaped channel 1446 (FIG. 30E) that can encompass a portion of the bolt head 1424. Further, the locking arm 1444 may have at least one cross-support 1448 that may pivotally couple the locking arm 1444 to the bolt head 1424 by passing through cammed aperture 1442.

The locking arm 1444 may also define at least one substantially semi-circular pivot face 1450 sized to correspond to a pivot pocket 1452 (FIG. 30D) created by the alignment plate 1432. When the locking arm 1444 is pivotally coupled to the bolt head 1424 by the cross-support 1448, the locking arm 1444 may pivot about a center axis 1453 defined by the pivot face 1450 and the pivot pocket 1452. As the locking arm 1444 pivots about the center axis 1453, the cross-support 1448 may contact the cammed aperture 1442 to cause the bolt head 1424 to move along the center axis 1430 away from the top surface of the second clamp 1404. As the bolt head 1424 moves farther from the top surface, the locking nut 1422 maintains the axial position of the lower surface of the first clamp member 1402. Accordingly, as the bolt head 1424 moves farther from the top surface of the second clamp 1404, the first and second clamps 1402, 1404 may become more compressed against the locking nut 1422. More specifically, the axial slots 1405 may allow for the openings 1412, 1418 to become more compressed as the locking arm 1444 forces the alignment plate 1432 towards the locking nut 1422.

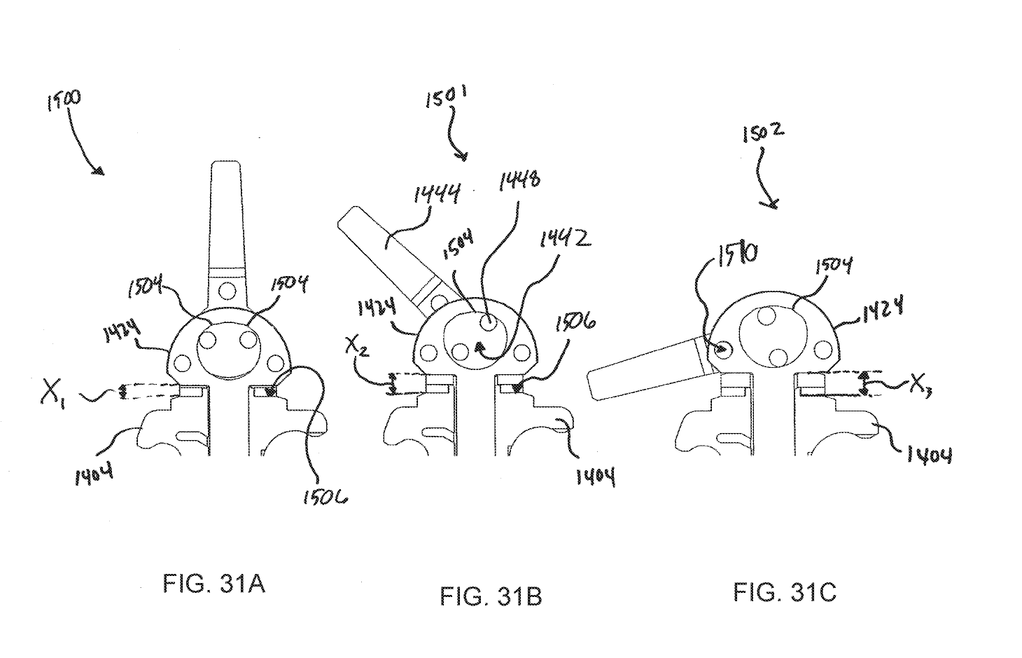

Referring now to FIGS. 31A, 31B, and 31C, the relations of the cammed aperture 1442 and the locking arm 1444 are more clearly illustrated in the cross sectional view in the fully opened position 1500, the partially compressed position 1501, and the fully compressed position 1502. More specifically, in the fully opened position 1500, the bolt head 1424 may be a distance X₁ from a top surface 1506 of the second clamp 1404. Accordingly, the first and second clamp members 1402 and 1404 may be in an uncompressed state, capable of receiving the various elongated members having a cylindrical shape, such as, for instance rods and pins.

In the partially compressed position 1501, the cross-support 1448 of the locking arm 1444 may become partially disposed within a pocket 1504 defined in the cammed aperture 1442. The pocket 1504 may be sufficiently sized to hold the cross support 1448 within the pocket 1504 without a user input. The pocket 1504 may be located at a radial position along the cammed aperture 1442 so that the bolt head 1424 is forced a distance X₂ from the top surface 1506 through the contact of the pivot face 1450 with the pivot pocket 1452. The distance X₂ may be greater than the distance X₁. Accordingly, in the partially compressed position 1501, the first and second clamp members 1402, 1404 may provide a provisional clamping force to the openings 1412, 1418.

In one embodiment, the partially compressed position 1501 may provide sufficient clamping pressure to any rods or pins that may be disposed in the openings 1412, 1418, to substantially restrict any movement of the pins or rods. The radial location of the pocket 1504, however, may be specifically determined to provide a compressing force that allows the rods or pins to be manipulated by supplemental force from a user but not by expected soft-tissue forces.

In the fully compressed position 1502, the bolt head 1424 may be separated a maximum distance X₃ from the top surface 1506, forcing the first and second clamp members 1402, 1404 into a fully compressed state against the locking nut 1422. In this position, any pins or rods disposed within the openings 1412, 1418 will be substantially locked in position.

The locking arm 1444 may also be locked in the fully compressed position by placing a pin or other locking member (not shown) through a locking through hole 1510. The locking through hole 1510 may be defined through a portion of both the bolt head 1424 and a portion of the locking arm 1444. When the locking arm 1444 is in the fully compressed position 1502, the through hole of the locking arm 1444 may become substantially concentric with the through hole of the bolt head 1424 so that the locking member can become disposed therein. Once the locking member is disposed within the locking through hole 1510, the locking arm 1444 may be substantially restricted from moving out of the fully compressed position.

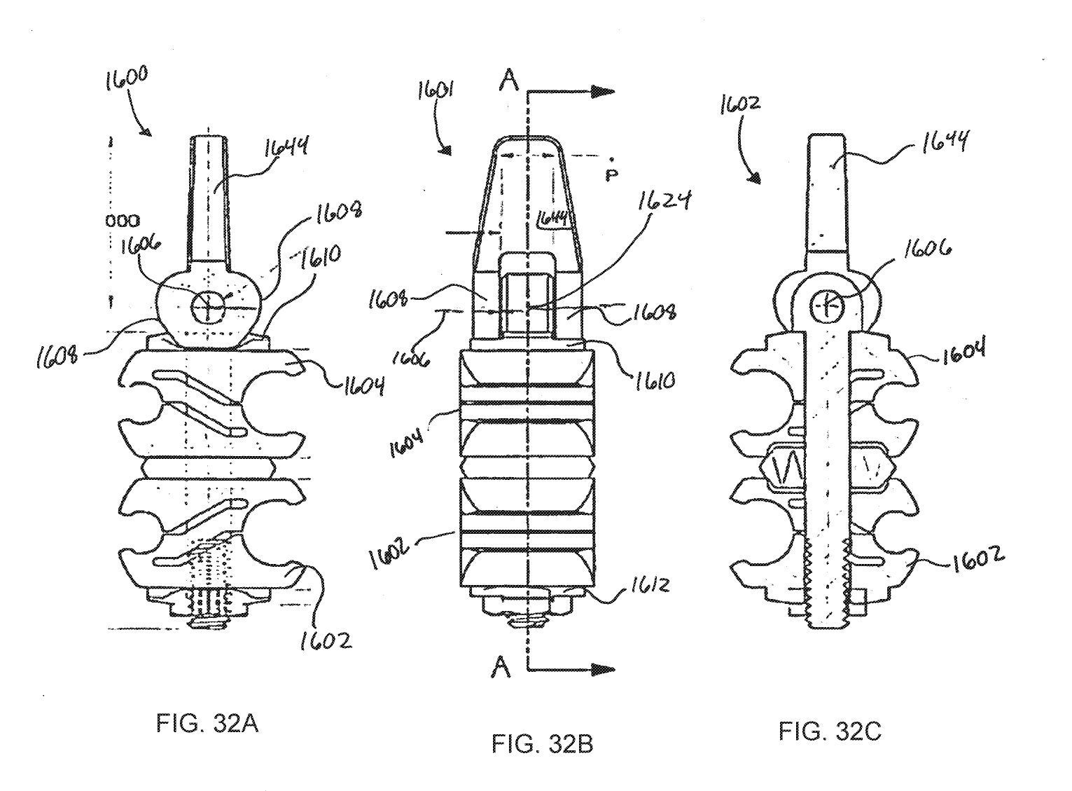

Referring now to FIGS. 32A, 32B, and 32C, a side view 1600, a front view 1601, and a cross section view 1603 is shown. This embodiment may have a substantially similar first and second clamp member 1602, 1604 as the previous embodiment, but may have a different compressing means. More specifically, a locking arm 1644 may be coupled to a bolt head 1624 through a pivot axis 1606. The locking arm 1644 may pivot between an uncompressed position 1600, a partially compressed position (not shown), and a fully compressed position (not shown). To achieve the various compression positions, the locking arm 1644 may have a cammed surface 1608 that contacts a spacer 1610 located on a top surface of the second clamp member 1602.

In one aspect of this embodiment, as the locking arm 1644 is transitioned from the fully opened position to the partially compressed position, the cammed surface 1608 travels along the spacer 1610 to increase the distance from the pivot axis 1606 to the top surface of the second clamp member 1602. In turn, the first and second clamp member 1602, 1604 can transition to the partially compressed state. In one embodiment, the cammed surface 1608 may be defined to allow the locking arm 1644 to be substantially maintained in the partially opened position without requiring a maintaining force by the user.

The locking arm 1644 may further transition from the partially compressed position to the fully compressed position. In the fully compressed position, the pivot axis 1606 may be spaced farther from the top surface of the second clamp member 1602 by the cammed surface 1608. Further, the cammed surface 1608 may pivot about the pivot axis 1606 to fully compress the first and second clamp members 1602, 1604 between the spacer 1610 and a bottom coupler 1612.

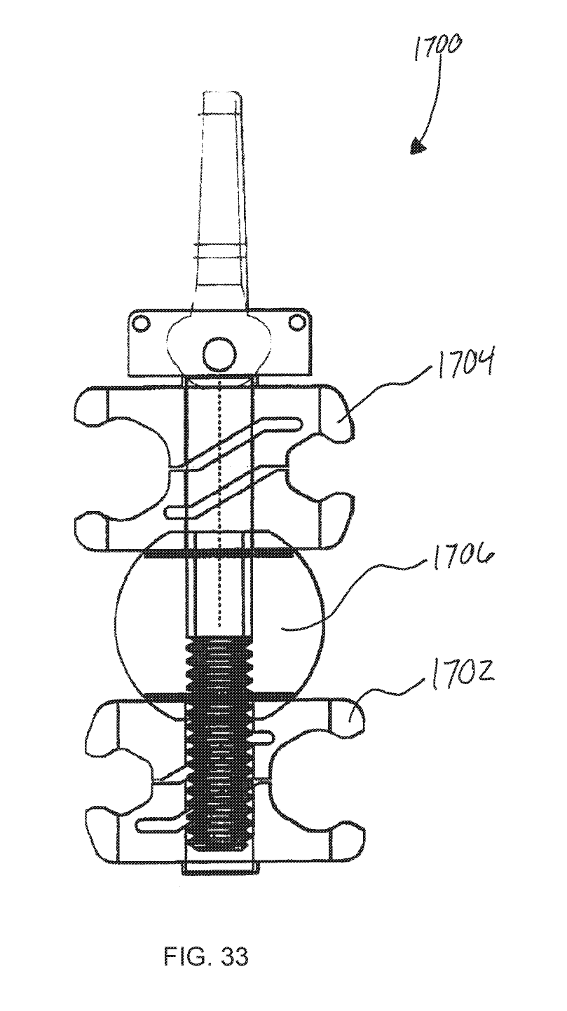

In accordance with another aspect of the present disclosure, an embodiment implementing a pivot ball 1700 is shown in FIG. 33. The embodiment implementing the pivot ball 1700 can be substantially similar to the embodiment shown and described for FIG. 32, with the addition of a pivot ball 1706 feature. The pivot ball 1706 may be located between a first and second clamp member 1702, 1704. By adding the pivot ball 1706 to the clamp members, additional flexibility in positioning of the pins or rods disposed therein may be achieved.

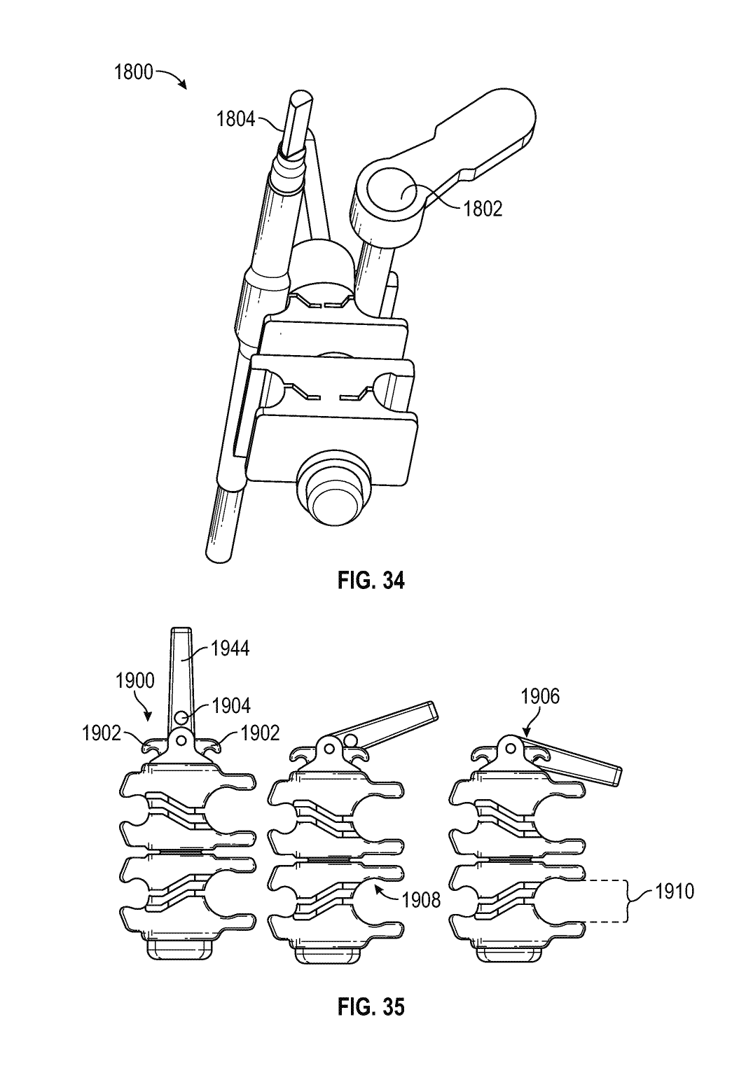

In yet another embodiment of this disclosure, a clamp assembly 1800 is shown in FIG. 34 with a drill sleeve 1802 and a pin 1804 disposed thereon. One advantage of this embodiment is that a user may clamp the drill sleeve 1802 to the assembly 1800 to guide a drill operation. After the drill operation, the drill sleeve may be removed and the pin 1804 may be positioned within the drilled area. Once the pin 1804 is positioned therein, the assembly 1800 may be rotated 180 degrees to snap onto the pin 1804. The clamp assembly 1800 may be positioned in a fully compressed position to retain the pin 1804 in the desired position.

A clamp assembly with a locking means 1900, in accordance with yet another aspect of the present disclosure is shown in FIG. 35. The clamp assembly with a locking means 1900 may be substantially similar to the embodiment of FIG. 32, with the addition of locking hooks 1902 and a locking through hole 1904. In this embodiment, the locking through hole 1904 may be disposed within a locking arm 1944. The locking through hole 1904 may be positioned along the locking arm 1944 at a location that allows the locking through hole 1904 to become substantially concentric with the locking hooks 1902 when the locking arm 1944 is in a fully locked position 1906. Further, the locking hooks 1902 may be located on two different sides of the locking arm 1944. In this configuration, the through hole 1904 may become concentric with one of the two locking hooks 1902 regardless of whether it is rotated in a clockwise or counterclockwise rotation to the fully locked position 1906.

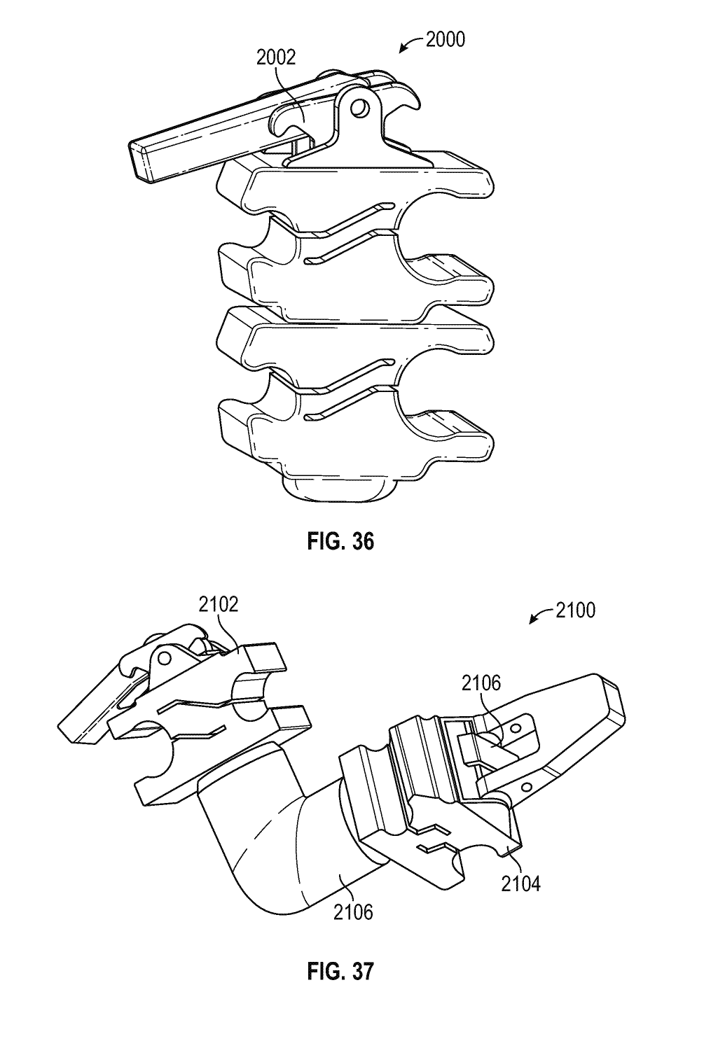

The assembly may be locked 2000 with a pin 2002 as shown in FIG. 36. More specifically, because the through hole 1904 and the locking hooks 1902 are substantially concentric when the locking arm 1944 is in the fully locked position 1906, the locking pin 2002 can be inserted therein to restrict the locking arm 1944 from moving out of the fully locked position 1906. The locking pin 2002 may have a means for being removeably coupled to the through hole 1904 when placed therein as is known in the art. In one non-limiting example, the locking pin may have an elastically deformable head that can provide a friction fit with the through hole 1904 when placed therein. A person having skill in the art would understand and appreciate that there are many ways a locking pin can be utilized to become removeably coupled to a through hole and this disclosure should not be limited to any one particular way.

An elbow clamp assembly 2100 is shown in FIG. 37. The elbow clamp assembly 2100 may have a first and second clamping assembly 2102, 2104 separated from one another by an elbow spacer 2106. The clamping assemblies 2102, 2104 may function in a substantially similar way as other clamping assemblies described herein except for they are coupled to the elbow spacer 2106 instead of to one another. One skilled in the art would understand how the clamping assembly 2102, 2104 could be removeably coupled to the elbow spacer 2106 in substantially the same way that they are coupled to one another. More specifically, a screw shaft may extend from a head down through a center aperture (not shown) of either clamp assembly 2102, 2104 to a coupling means (not shown) within the elbow spacer 2106. In one embodiment, the coupling means could be a threaded nut designed to correlate to the threaded screw shaft but one skilled in the art would understand the plurality of coupling methods that could be used.

The elbow spacer 2106 could also contain a serrated face that can be correspondingly coupled to a serrated face located on the clamping assemblies 2102, 2104 as has been explained in more detail above. One non-exhaustive use of the elbow spacer 2106 is to create substantially spherical reach for any pins or rods that may be coupled to the clamp assemblies 2102, 2104. A person having skill in the art understands that by creating two rotational axis 90 degrees offset from one another allows for a substantially spherical field of reach for anything coupled thereto.

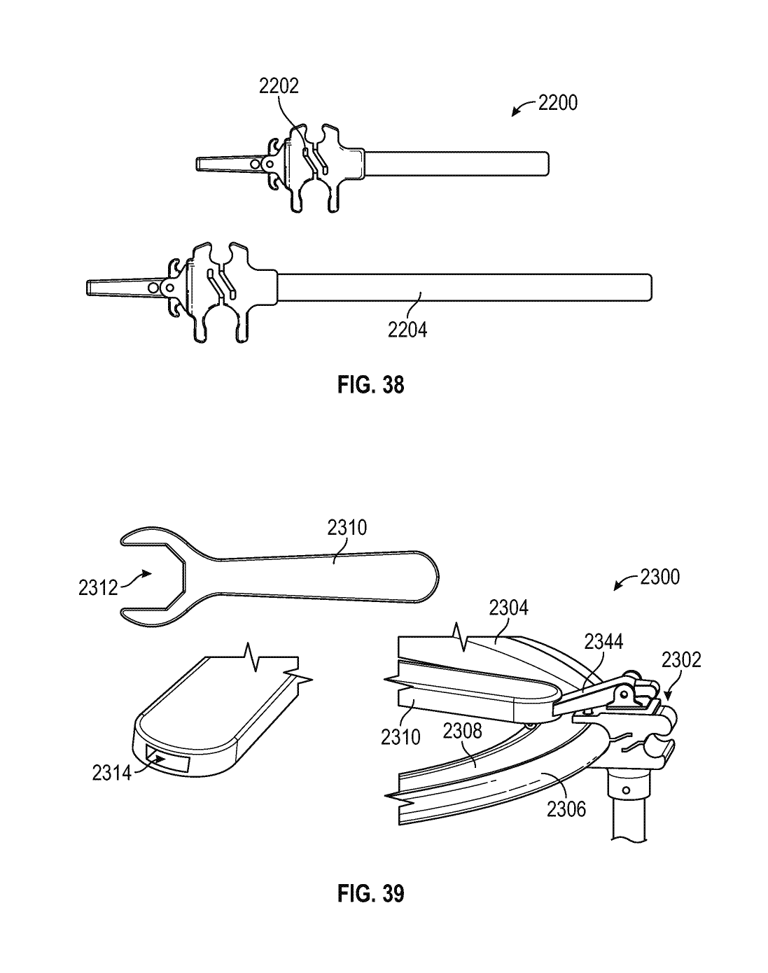

The shaft clamp assembly 2200 shown in FIG. 38 may have a substantially similar clamping assembly 2202 as the elbow clamp assembly 2100 of FIG. 37. The shaft clamp assembly 2200, however, may be coupled to a shaft 2204 in a similar way as described above for the elbow clamp assembly 2100. Alternatively, the clamping assembly 2202 may not be rotatably coupled to the shaft 2204. In one embodiment, the entire shaft clamp assembly 2200 can rotate about an axis defined by the shaft 2204. In this embodiment, it may be unnecessary to implement the serrated pivotal connection between the base of the clamping assembly 2202 and the shaft 2204 because the entire shaft clamp assembly 2200 may rotate.

In one embodiment, a clamping assembly 2302 may be coupled to a frame 2304 instead of a rod or pin 2300 as shown in FIG. 39. The above teachings of a clamping mechanism can be substantially incorporated to allow for removeably coupling the clamp assembly 2302 to the frame 2304. In this embodiment, the frame 2304 may have a semicircular edge 2306 coupled to at least one substantially planar frame element 2308. The semicircular edge 2306 may be defined by an arc that is greater than 180 degrees. Further, the diameter of the arc may be greater than the thickness of the planar frame element 2308. In this configuration, the semicircular edge may correspond with the dimensions of an opening 1908 (FIG. 35) for the clamping assembly 2302. Further, the opening 1908 may have a mouth 1910 (FIG. 35) that is dimensioned to be substantially the same width as the thickness of the planar frame element 2308.

In this configuration, when the clamping assembly 2302 is in an uncompressed orientation, the opening 1908 may be expandable to encompass the semicircular edge 2306. Further, the clamping assembly 2302 may be transitioned to a fully compressed position while coupled to the frame 2304. Once transitioned to the fully compressed position. The clamp assembly 2302 may become substantially fixed to the frame 2304.

While a particularly shaped frame has been described in detail above, this disclosure should not be limited to the particular sizes described herein. A person having skill in the relevant art understands that a plurality of different sized frames and clamping mechanism could be utilized without straying from the teachings of this disclosure. Accordingly, this disclosure should not be limited to any one configuration.

In yet another aspect of the embodiment of FIG. 39, a multipurpose tool 2310 is shown. The tool 2310 may have a spaced end 2312 that is designed to receive and manipulate a portion of a desired object (not shown). The tool 2310 may also define a cavity 2314 that correlates with the size and shape of a portion of a locking arm 2344 of the clamp assembly 2302. The cavity 2314 may be sufficiently sized to allow a substantial portion of the locking arm 2344 to become disposed therein. Once the locking arm 2344 is disposed within the cavity 2314, the tool 2310 may act as a lever arm to allow the user to more easily transition the clamp assembly 2302 from the uncompressed position to the compressed position.

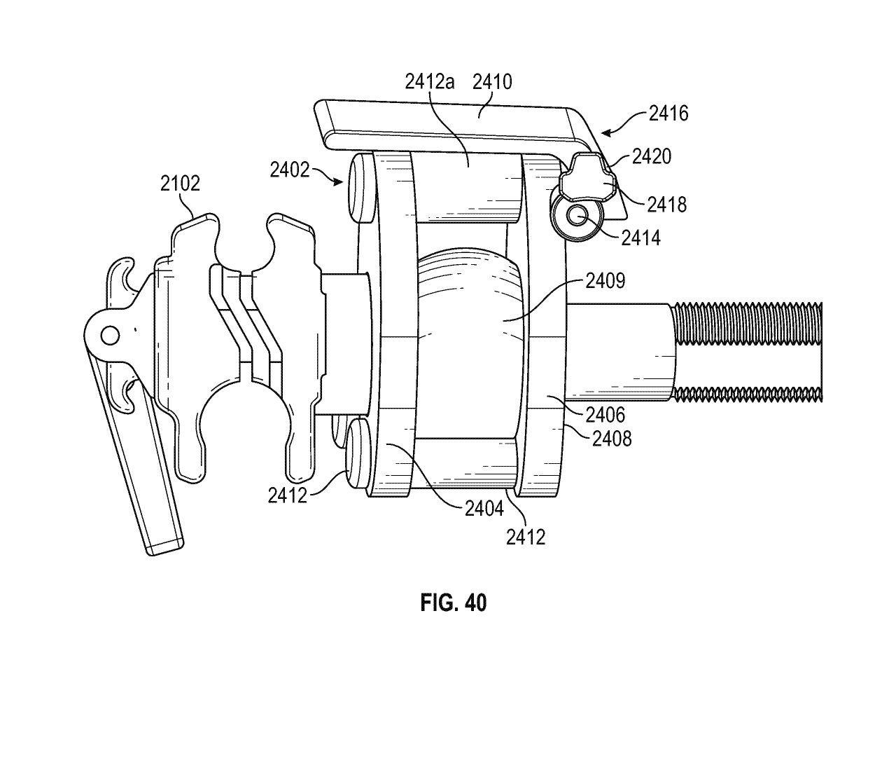

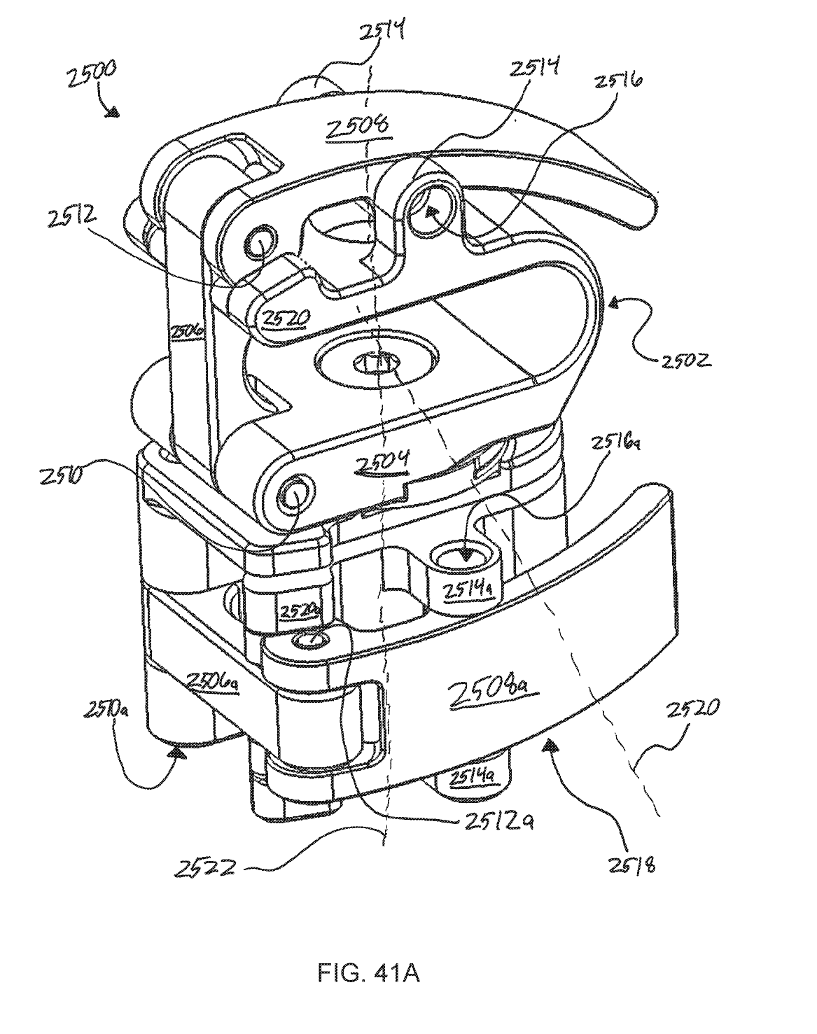

As shown in FIG. 40, the poly-axial pivot housing 2402 includes a top plate 2404, a bottom plate 2406, a sphere/post assembly 2408 and a locking arm 2410. The top plate 2404 is configured to connect to a clamping assembly (clamping assembly 2102, for example) by threading the clamp into a threaded bore hole (not shown) of the top plate 2404. When the housing 2402 is assembled, the top plate 2404 provides a compressive loading along with the bottom plate 2406 to thereby hold and center the sphere/post assembly's spherical body 2409 therebetween. To hold and center the spherical body 2409 in response to the compressive loading of the top plate 2404, the bottom plate 2406 includes a substantially circular recessed cavity (not shown) that is dimensioned to accept the top portion of the spherical body 2409 as the pivot housing 2402 is locked relative to the frame. The bottom plate 2406 includes a substantially circular recessed cavity (not shown) that is dimensioned to accept the bottom portion of the spherical body 2409 as the housing is locked.

The top ring also includes three clearance holes adapted to receive locking bolts 2412 that pass through the top plate 2404 and thread into the bottom plate 2406 via corresponding threaded holes. Because of the threaded relationship between the locking bolts 2412 and the bottom plate 2406, the locking bolts 2412 may allow the poly-axial pivot housing 2402 to be provisionally locked into place in response to the compressive forces and tension applied to the spherical body 2409 by the top and bottom plates 2404, 2406 (i.e., it achieves a friction fit). Specifically, the provisional lock is obtained when the top and bottom plates 2404, 2406 are positioned so that the compressive force is applied to the spherical body 2409 to prevent movement by soft-tissue forces. The friction fit and soft tissue tension allows the surgeon to provisionally lock the frame and make minor adjustments to the bone alignment. Once the surgeon is satisfied with the fixation of the bone fragments, the frame can then be definitively locked to maintain the bone fragments within the desired alignment.

To definitively lock the housing 2402, in accordance with certain embodiments herein, one of the locking bolts 2412a is connected to the locking arm 2410 by way of a cammed relationship between the bottom plate and the locking arm 2410. The locking arm 2410 may have a cammed profile about a locking arm pin 2414. When the locking arm 2410 is in the locked position 2416 as shown in FIG. 40, the cammed profile may separate the pin 2414 from the bottom plate 2406, compressing the spherical body 2409 between the top and bottom plates 2404, 2406.

In accordance with certain aspects of the present disclosure, the pivot housing 2402 may further include a locking pin 2418 that is configured to hold the locking arm 2410 in place once it has been definitively locked. According to this embodiment, the bottom plate 2406, together with the locking arm 2410, includes a series of aligned through-holes that are configured to receive the locking pin 2418 as it is inserted therethrough. Specifically, the locking pin 2418 is inserted into a through-hole of the bottom plate 2406 from its outer surface, and particularly in such a manner that the locking pin 2418 continues to pass through the through-hole of the locking arm (not particularly shown). The locking pin 2418 may include a head portion 2420 on one end and a locking mechanism (not shown) on its opposite end. The head portion 2420 is dimensioned in such a manner that it is unable to enter the through-hole of the bottom plate 2406 as the locking pin 2418 is inserted therein. Since the head portion 2420 has a circumferential dimension larger than that of the through-hole, when the locking pin 2418 is inserted therethrough, the head portion 2420 is configured to serve as a stop surface as it flushingly engages the outer surface of the bottom plate 2406. While various different locking arrangements may be utilized in accordance with the present disclosure, in accordance with certain illustrative aspects herein, the locking pin 2418 may be manufactured from a material that is compressible or elastically deformable (e.g., plastic, rubber). In accordance with this illustrative aspect, the locking pin 2418 may include a slightly flared portion that is configured to compress as it passes through the through-hole, yet expand back to its original dimensions after passing fully therethrough. Once returning to its original dimensions, the locking pin 2418 will expand to a size that is slightly larger than the dimensions of the through-hole, thereby locking it into place relative thereto.

Extending from the spherical body 2409 is an attachment post 2422 that is configured to attach the pivot housing 2402 to a frame connector or ratcheting strut. While the attachment post 2422 and the spherical body 2409 can be manufactured as a single component or unit (e.g., overmolded into a single unit), in accordance with certain aspects of the present disclosure, the attachment post 2422 and the spherical body 2409 may be independent of one another. For instance, the spherical body 2409 may include a threaded aperture (not shown) that is configured to receive a threaded portion (not shown) of the attachment post 2422.

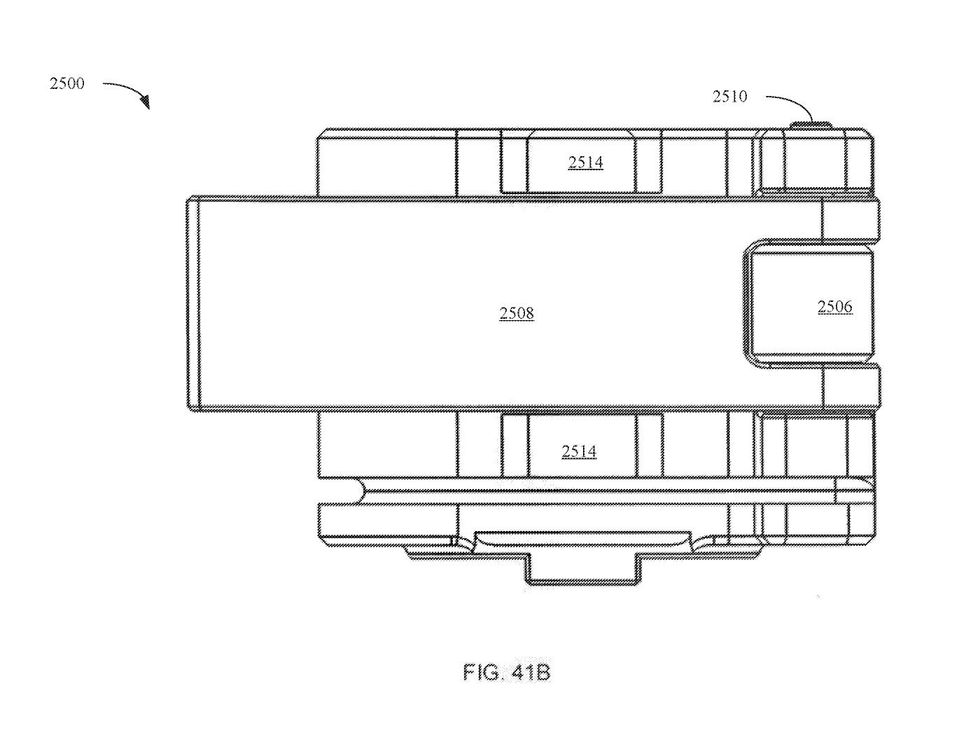

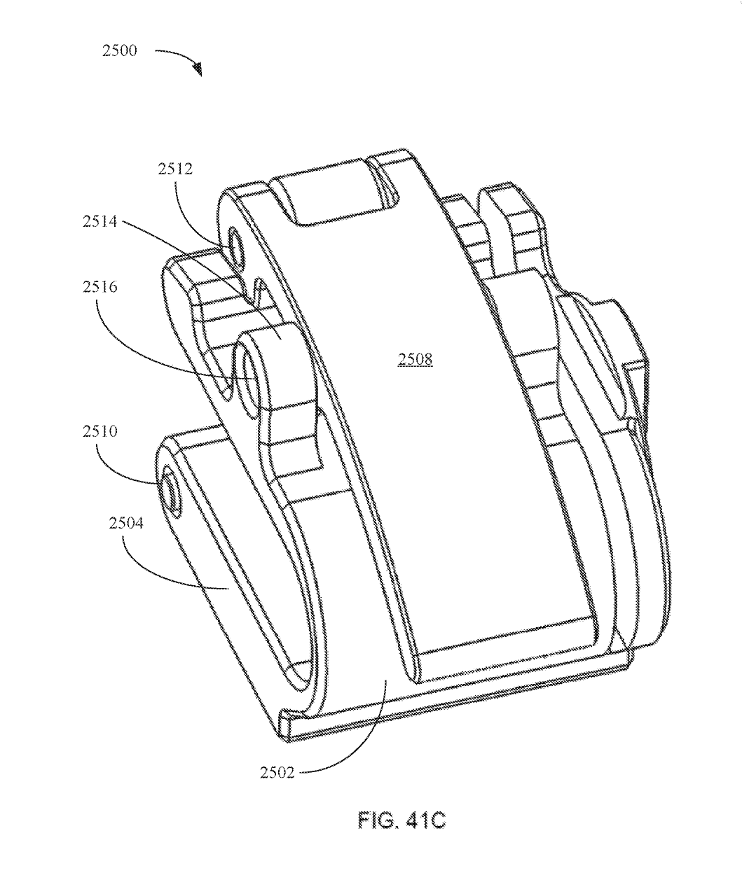

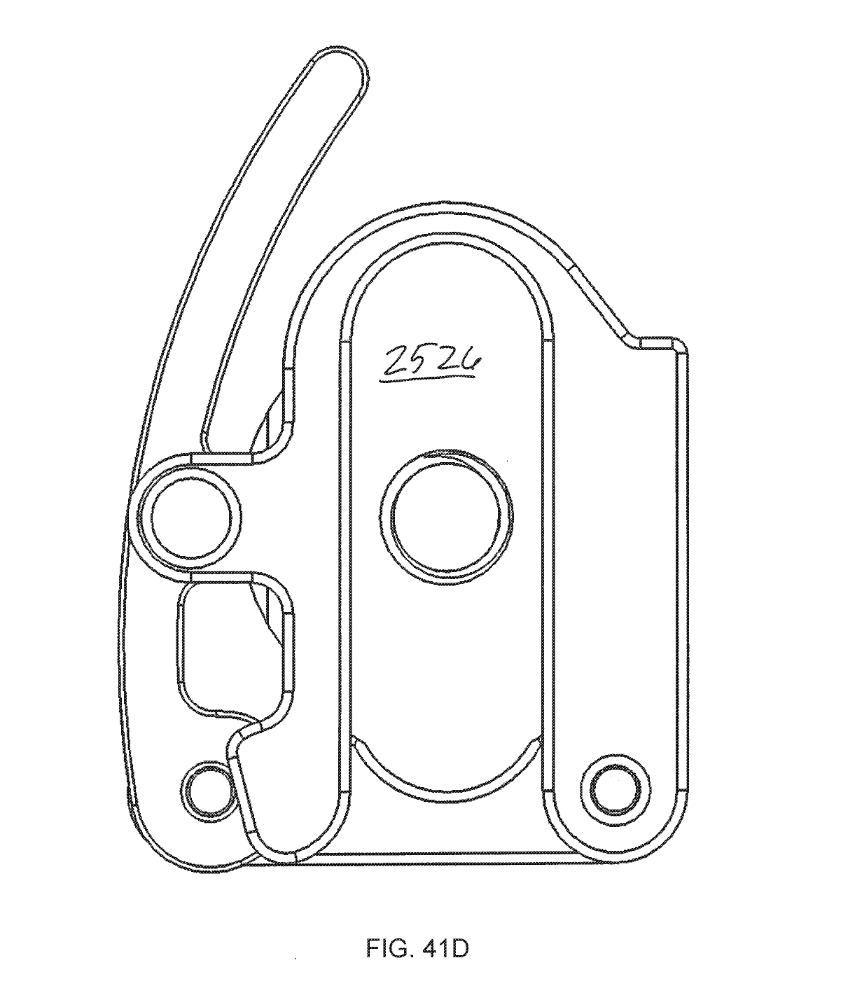

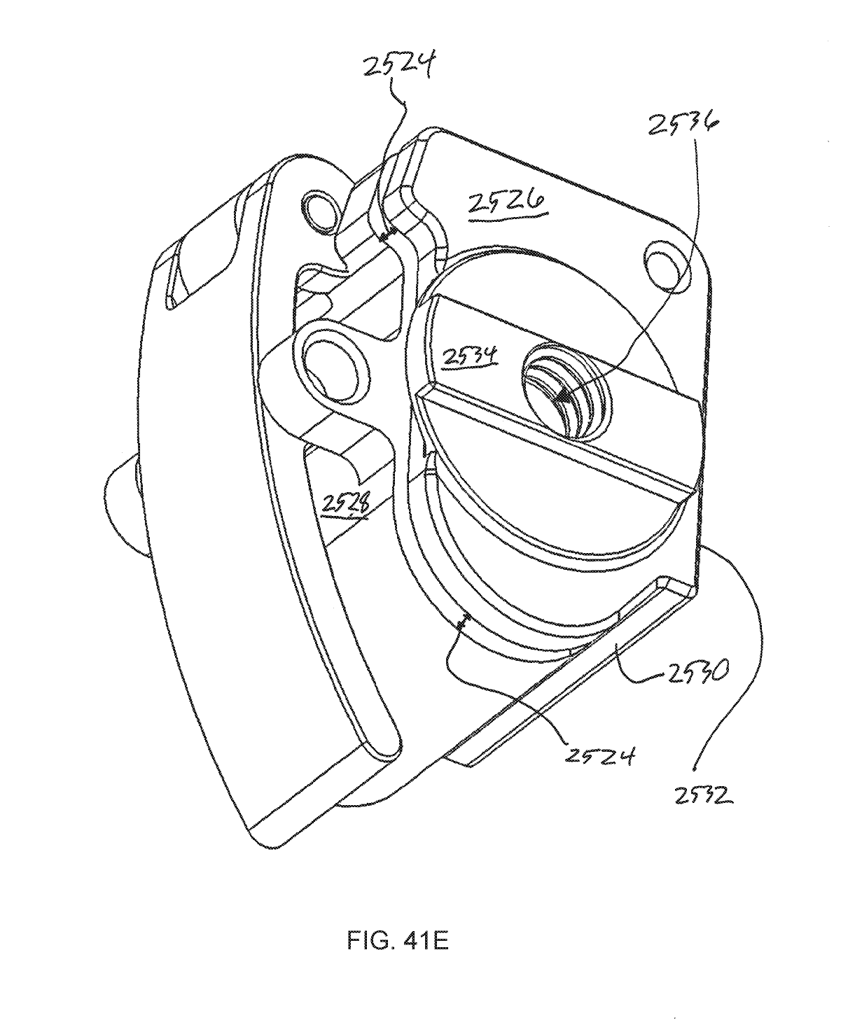

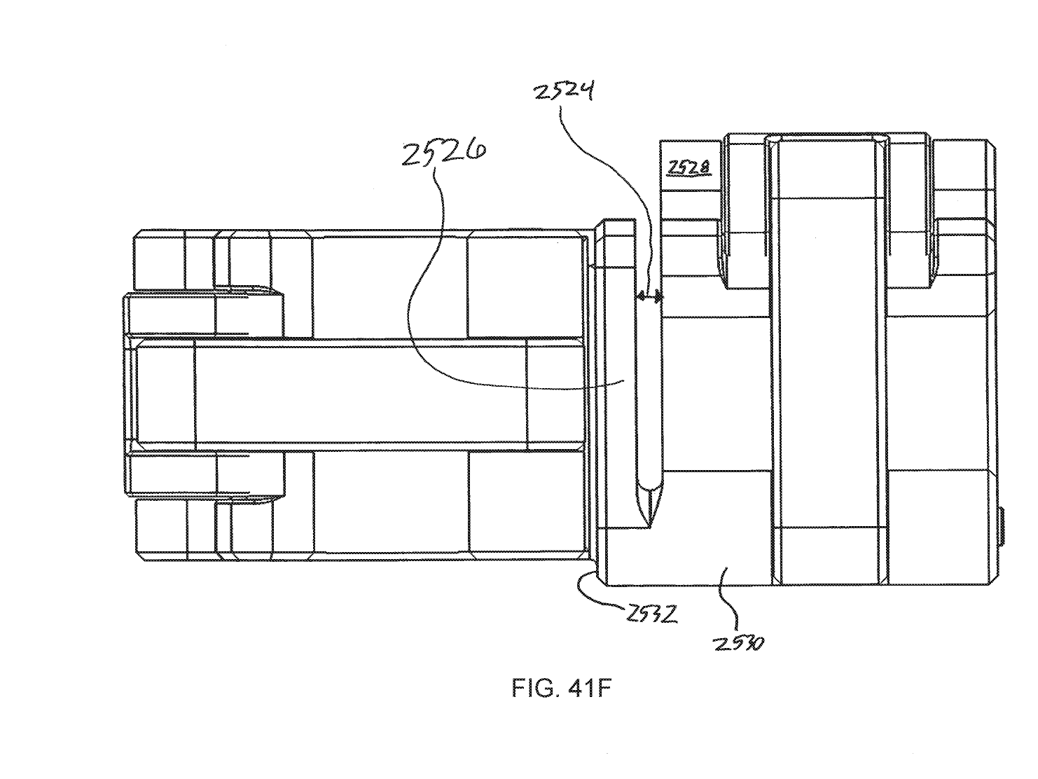

FIGS. 41A-41F depict another illustrative clamping assembly 2500 in accordance with one embodiment of the present teachings. In accordance with this aspect of the present disclosure, and with specific reference to FIG. 41A, the clamping assembly 2500 may be comprised of a top clamp assembly 2502 and a bottom clamp assembly 2518. The top clamp assembly 2502 may include a clamp body 2504, a locking arm 2506, a cam arm 2508, a locking arm pivot pin 2510 and a cam arm pivot pin 2512. The clamping assembly 2500 is configured to be snapped onto a ring frame, or a rod (not shown) by positioning the clamp body 2504 substantially perpendicular to the ring leg or rod and applying pressure to force end jaws 2520 open and over the ring or rod. Alternatively, the clamping assembly 2500 can be snapped onto the ring frame or rod by positioning the clamp body 2504 at the end of a ring leg or rod and applying pressure to slide the body onto the ring or rod.

Once positioned on the ring frame or rod, the locking arm 2506 compresses and centers the ring frame or rod inside the clamp body 2504. To achieve this, the clamp may be provisionally locked by rotating the locking arm 2506 (via the locking arm pivot pin 2510) towards and into a cam arm pocket (not particularly shown in this embodiment). Once the surgeon is satisfied with the position and fixation, the frame or fixator is definitively locked without the use of additional tools or equipment. To achieve the definitive lock, the cam arm 2508 is rotated towards the clamp body 2504 via the cam arm pivot pin 2512 located at the center of the cam arm 2508 until it touches the clamp body 2504.

Once the cam arm 2508 is positioned against the clamp body 2504 during a definitive locking process, in accordance with certain aspects of the present disclosure, the cam arm 2508 can be further locked into place by utilizing a locking pin (not shown) that is configured to be inserted through the cam arm 2508. According to this embodiment, the clamp body 2504 has a pair of upwardly projecting tabs 2514, each having a through-hole 2516 formed therein. When the cam arm 2508 is positioned against the clamp body 2504, the through-holes 2516 align with a through-hole formed into the cam arm (not particularly shown this embodiment) such that a common through-hole is created. The locking pin can then be inserted through this common through-hole, thereby preventing the cam arm 2508 from being lifted away from the clamp body 2504 until the locking pin is first removed.

The bottom clamp assembly 2518 may provide substantially similar clamping features as described above for the top clamp assembly 2502. More specifically, the bottom clamp assembly 2518 may have a locking arm 2506a, a cam arm 2508a, a locking arm pivot pin 2510a, and a cam arm pivot pin 2512a. The bottom clamp assembly 2518 may also comprise a pair of upwardly projecting tabs 2514a that define a through hole 2516a. The clamping and locking features of the bottom clamp assembly 2518 may function substantially similarly to the clamping and locking features described above for the top clamp assembly 2502.

One aspect of the bottom clamp assembly 2518 that is different from the top clamp assembly 2502 is the ability of the bottom clamp assembly 2518 to be coupled to the top clamp assembly 2502 at a side location. The embodiment shown in FIG. 41A illustrates how a ring frame or a rod can be coupled to the top clamp assembly 2502 along a first direction 2523 while a ring frame or rod can simultaneously be coupled to the bottom clamp assembly 2518 along a second direction 2522. The first and second directions 2523, 2522, may be substantially perpendicular to one another.

In one embodiment, allowing for a perpendicular coupling of two frame elements may allow for a separate plane to be created for fixation. FIG. 41E illustrates with more detail how the bottom clamp assembly 2518 may allow for sufficient clamping deformation. More specifically, a gap 2524 may be defined between a side portion 2526 and a top portion 2528 of the bottom clamp assembly 2518. The gap 2524 may allow the top portion 2528 to move relative to, and independent from, the side portion 2526. The gap 2524 may be necessary to allow for adequate deformation of the top portion 2528 to provide a sufficient compressive force for the definitively locked position.

The side portion 2526 may be coupled to a bottom portion 2530 along a first edge 2532. The side portion 2526 may be sufficiently coupled to the bottom portion 2530 to provide for a location to removeably couple the bottom clamp 2518 to the top clamp assembly 2502. Further, the side portion 2526 may provide for a coupling location for any number of clamping members or devices used for external fixation.

The side portion 2526 may also define a ridge 2534 defined thereabove. The ridge 2534 may further define a through hole 2536 defined to allow for removeably coupling the bottom clamp assembly 2518 to the top clamp assembly 2502. The ridge 2534 may be shaped to be received by a corresponding valley (not shown) in the top clamp assembly 2502. When the ridge 2534 is coupled to the valley, the top clamp assembly 2502 may be pivotally coupled to the bottom clamp assembly 2518. That is to say, the top clamp assembly 2502 may be substantially restricted from moving radially relative to the bottom clamp assembly 2518 because the valley may substantially encompass the ridge 2534.

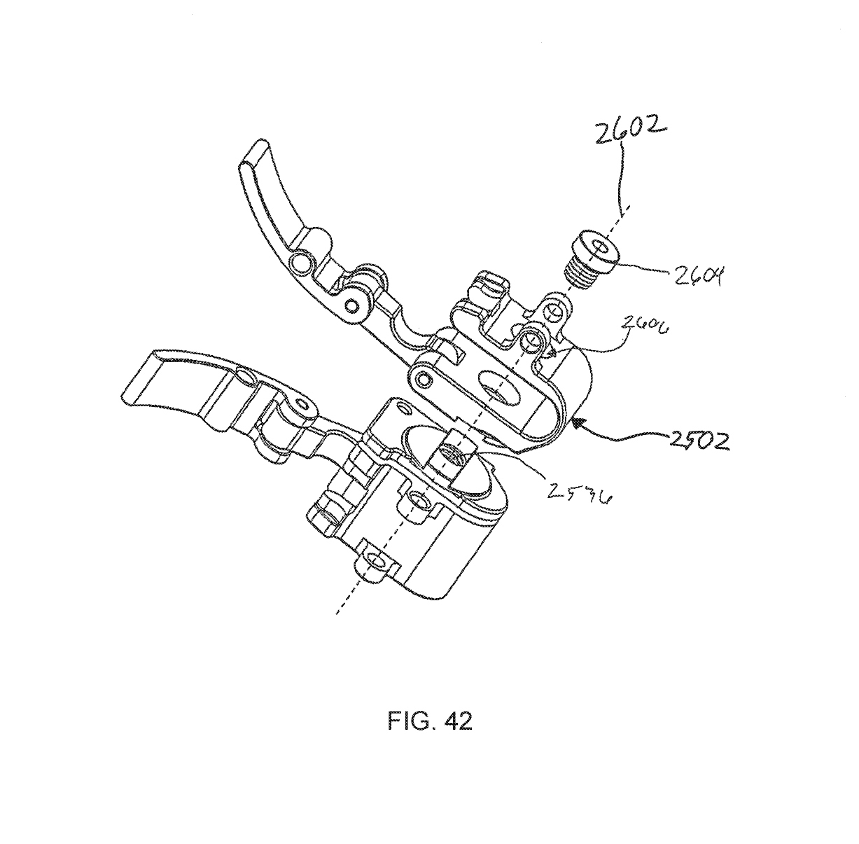

An exploded view 2600 of the top and bottom clamp assemblies 2502, 2518 is shown in FIG. 42. In the exploded view 2600, the top and bottom clamp assemblies 2502, 2518 are separated from one another along an assembly axis 2602. A coupling member 2604 is more clearly shown in the exploded view 2600. The coupling member 2604 may pass through an upper aperture 2606 to couple the top clamp assembly 2502 to the bottom clamp assembly 2518. More specifically a bottom aperture 2608 may be sized to allow the head of the coupling member 2604 to become partially disposed therein while not allowing the head to pass fully therethrough. The shaft of the coupling member 2604 may be sufficiently long to engage the through hole 2536 but sufficiently short to keep from entering the clamping area of the bottom clamp assembly 2518. Accordingly, the coupling member 2604 can substantially couple the top and bottom clamp assemblies 2502, 2518 to one another without substantially affecting the functionality of the clamping members. Further, the coupling member 2604 may hold the ridge 2534 in alignment with the corresponding valley to restrict radial movement between the top and bottom clamp assemblies 2502, 2518.

Another function of the side portion 2526 may be to act as a stop for the frame member or other object being clamped therein. While the top clamp assembly 2502 may allow for the object to be clamped to pass substantially therethrough, the bottom clamp assembly 2518 restricts the available positions for the object to be clamped therein. As is shown in the side view 2550 of FIG. 41D, the side portion 2526 may contact the object being clamped therein once it is fully seated within the bottom clamp assembly 2518. Once the object being clamped is fully seated against the side surface 2562, the bottom clamp assembly 1518 may be definitively clamped to the object.

While an exemplary embodiment incorporating the principles of the present application has been disclosed hereinabove, the present application is not limited to the disclosed embodiments. Instead, this application is intended to cover any variations, uses, or adaptations of the application using its general principles. Further, this application is intended to cover such departures from the present disclosure as come within known or customary practice in the art to which this present application pertains and which fall within the limits of the appended claims.

The terminology used herein is for the purpose of describing particular illustrative embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms such as “inner”, “outer”, “beneath”, “below”, “lower”, “above”, “upper” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations).

Claimed is:

1. An external fixation system comprising:
a ring frame;
a strut; and
a pivot housing connecting the strut to the ring frame, the pivot housing comprising:
a first compression member attached to the ring frame,
a second compression member spaced from the first compression member and attached to the first compression member, and
a post assembly connected to the strut via an attachment post protruding from a spherical component and passing through the first compression member, the spherical component compressed between the first compression member and the second compression member to retain the post assembly in a fixed position.

2. The external fixation system of claim 1, further comprising a socket configured to rotate the second compression member, the socket including an internal structure that mates with external features of the second compression member to enable rotation of the second compression member when the socket rotates.

3. The external fixation system of claim 1, wherein the first compression member includes a platform extending from a first surface, the platform sized to receive a clamping assembly.

4. The external fixation system of claim 1, further comprising a locking member movable from a first position to a second position, wherein when the locking member is in the first position the post assembly is pivotable within a space defined by the first compression member and the second compression member and when the locking member is in the second position the compression force is applied to the spherical component.

5. The external fixation system of claim 1, wherein the first compression member is connected to the second compression member via a plurality of threaded members.

6. The external fixation system of claim 1, further comprising:
a crossbar connected to the ring frame via a pair of distraction extension arms sized to space the crossbar a fixed distance from the ring frame; and
a first distraction handle and a second distraction handle connected to the crossbar and configured to allow a user to reposition the external fixation system once attached to a patient.

7. The external fixation system of claim 6, wherein the first distraction handle and the second distraction handle are each pivotably connected to the crossbar.

8. An external fixation system comprising:
a proximal ring frame to receive a first set of one or more fixation elements;
a distal ring frame to receive a second set of one or more fixation elements;
a first frame strut to fix a first relative position between the proximal ring frame and the distal ring frame;
a first pivot housing pivotably coupling the proximal ring frame and the first frame strut; and
a second pivot housing pivotably coupling the distal ring frame and the first frame strut.

9. The external fixation system of claim 8, further comprising:
a second frame strut to fix a second relative position between the proximal ring frame and the distal ring frame;
a third pivot housing pivotably coupling the proximal ring frame and the second frame strut; and
a fourth pivot housing pivotably coupling the distal ring frame and the second frame strut.

10. The external fixation system of claim 8, wherein the first pivot housing and the second pivot housing each comprise: a post assembly connected to the first frame strut and a locking arm located between a first ring and a second ring, the locking arm configured to lock the post assembly in a fixed position.

11. The external fixation system of claim 8, wherein the proximal ring frame or the distal ring frame includes a platform extending from a first surface, the platform sized to receive a clamping assembly.

12. The external fixation system of claim 8, wherein the first frame strut includes a ratcheting member configured to fix a length of the first frame strut.

13. The external fixation system of claim 8, further comprising:
a crossbar connected to the proximal ring frame or the distal ring frame via a pair of distraction extension arms; and
a first distraction handle and a second distraction handle connected to the crossbar.

14. The external fixation system of claim 13, wherein the first distraction handle and the second distraction handle are each pivotably connected to the crossbar.

15. An external fixation system comprising:
a proximal ring frame;
a distal ring frame;
a first pivot housing pivotably connecting the proximal ring frame to a first frame strut; and
a second pivot housing pivotably connecting the distal ring frame to the first frame strut,
wherein the first pivot housing and the second pivot housing each include:
a clamp body sized to fit onto the proximal ring frame or the distal ring frame, and
a locking arm connected to the clamp body and pivotable about a point on the clamp body, wherein when the locking arm is in a first position the clamp body is slideable along a portion of the proximal ring frame or the distal ring frame and when the locking arm is in a second position a portion of the clamp body contacts the proximal ring frame or the distal ring frame to secure the clamp body in a fixed location.

16. The external fixation system of claim 15, wherein the frame strut is selected from a plurality of frame struts, each of the plurality of frame struts having a different size.

17. The external fixation system of claim 15, wherein the frame strut is polyaxially connected to the proximal ring frame and the distal ring frame via the first pivot housing and the second pivot housing.

18. The external fixation system of claim 15, further comprising:
a crossbar connected to the proximal ring frame or the distal ring frame via a pair of distraction extension arms that space the cross bar from the proximal ring frame or the distal ring frame a fixed distance; and
a first distraction handle and a second distraction handle connected to the crossbar, the first distraction handle and the second distraction handle configured to allow a user to reposition the external fixation system once attached to a patient.

19. The external fixation system of claim 18, wherein the first distraction handle and the second distraction handle are each pivotably connected to the crossbar.

20. The external fixation system of claim 15, further comprising:
a second frame strut to fix a second relative position between the proximal ring frame and the distal ring frame;
a third pivot housing pivotably coupling the proximal ring frame and the second frame strut; and
a fourth pivot housing pivotably coupling the distal ring frame and the second frame strut.
